(19) United States
(12) Patent Application Publication
(10) Pub. No.: US 2024/0158450 A1
(43) Pub. Date: May 16, 2024

(54) METHODS AND COMPOSITIONS FOR ANTIBODY-EVADING VIRUS VECTORS

(71) Applicants: The University of North Carolina at Chapel Hill, Chapel Hill, NC (US); University of Florida Research Foundation, Inc., Gainesville, FL (US)

(72) Inventors: Aravind Asokan, Chapel Hill, NC (US); Mavis Agbandje-McKenna, Gainesville, FL (US); Long Ping Victor Tse, Carrboro, NC (US); Brittney Gurda, Collegeville, PA (US)

(21) Appl. No.: 18/488,604

(22) Filed: Oct. 17, 2023

Related U.S. Application Data
(63) Continuation of application No. 17/526,510, filed on Nov. 15, 2021, now Pat. No. 11,840,555.
Application No. 17/526,510 is a continuation of application No. 16/921,239, filed on Jul. 6, 2020, now Pat. No. 11,208,438.
Application No. 16/921,239 is a continuation of application No. 15/763,706, filed on Mar. 27, 2018, now Pat. No. 10,745,447.
(60) Provisional application No. 62/234,016, filed on Sep. 28, 2015.

(51) Int. Cl. C07K 14/005; C07K 16/08; C12N 15/86

(52) U.S. Cl. CPC C07K 14/005; C07K 16/081; C12N 15/86; A61K 2039/507; C07K 2317/34; C12N 2750/14122

(57) ABSTRACT

The present invention provides AAV capsid proteins comprising a modification in the amino acid sequence and virus vectors comprising the modified AAV capsid protein. The invention also provides methods of administering the virus vectors and virus capsids of the invention to a cell or to a subject in vivo.

### STATEMENT OF PRIORITY

This application is a continuation application of U.S. application Ser. No. 17/526,510, filed Nov. 15, 2021, which is a continuation application of U.S. application Ser. No. 16/921,239, filed Jul. 6, 2020, now U.S. Pat. No. 11,208,438, issued Dec. 28, 2021, which is a continuation of U.S. application Ser. No. 15/763,706, filed Mar. 27, 2018, now U.S. Pat. No. 10,745,447, issued Aug. 18, 2020, which is a 35 U.S.C. § 371 national phase application of International Application Serial No. PCT/US2016/054143, filed Sep. 28, 2016, which claims the benefit, under 35 U.S.C. § 119(e), of U.S. Provisional Application No. 62/234,016, filed Sep. 28, 2015, the entire contents of each of which are incorporated by reference herein.

### STATEMENT OF GOVERNMENT SUPPORT

This invention was made with government funding under Grant Nos. HL112761, HL089221 and GM082946 awarded by the National Institutes of Health. The government has certain rights in the invention.

### STATEMENT REGARDING ELECTRONIC FILING OF A SEQUENCE LISTING

A Sequence Listing in XML format, entitled 5470-752CT3 ST26.xml, 446,393 bytes in size, generated on Oct. 17, 2023, and filed herewith, is hereby incorporated by reference in its entirety for its disclosures.

### FIELD OF THE INVENTION

The present invention relates to modified capsid proteins from adeno-associated virus (AAV) and virus capsids and virus vectors comprising the same. In particular, the invention relates to modified AAV capsid proteins and capsids comprising the same that can be incorporated into virus vectors to confer a phenotype of evasion of neutralizing antibodies without decreased transduction efficiency.

### BACKGROUND OF THE INVENTION

Host-derived pre-existing antibodies generated upon natural encounter of AAV or recombinant AAV vectors prevent first time as well as repeat administration of AAV vectors as vaccines and/or for gene therapy. Serological studies reveal a high prevalence of antibodies in the human population worldwide with about 67% of people having antibodies against AAV1, 72% against AAV2, and about 40% against AAV5 through AAV9.

Furthermore, in gene therapy, certain clinical scenarios involving gene silencing or tissue degeneration may require multiple AAV vector administrations to sustain long term expression of the transgene. To circumvent these issues, recombinant AAV vectors which evade antibody recognition (AAVe) are required. This invention will help a) expand the eligible cohort of patients suitable for AAV-based gene therapy and b) allow multiple, repeat administrations of AAV-based gene therapy vectors.

The present invention overcomes previous shortcomings in the art by providing methods and compositions comprising an adeno-associated virus (AAV) capsid protein, comprising one or more amino acid substitutions, wherein the substitutions introduce into an AAV vector comprising these modified capsid proteins the ability to evade host antibodies.

### SUMMARY OF THE INVENTION

In one aspect, the present invention provides an adeno-associated virus (AAV) capsid protein, comprising one or more amino acids substitutions, wherein the substitutions modify one or more previously existing antigenic sites on the AAV capsid protein.

In some embodiments, the amino acid substitutions are in antigenic footprints identified by peptide epitope mapping or cryo-electron microscopy studies of AAV-Antibody complexes containing capsids based on AAV1, AAV3, AAV4, AAV5, AAV6, AAV7, AAV 8, AAV9, AAV10, AAV11, AAV12, AAVrh8, AAVrh10, AAVrh32.33, Avian AAV or Bovine AAV.

In some embodiments, the modified antigenic site can prevent antibodies from binding or recognizing or neutralizing AAV capsids, wherein the antibody is an IgG (including IgG1, IgG2a, IgG2b, IgG3), IgM, IgE or IgA.

In some embodiments, the modified antigenic site can prevent binding or recognition or neutralization of AAV capsids by antibodies from different animal species, wherein the animal is human, canine, feline or equine.

In some embodiments, the modified antigenic site is a common antigenic motif, wherein a specific antibody or a cross-reactive antibody can bind, recognize or neutralize the AAV capsid.

In some embodiments, the substitutions introduce a modified antigenic site from a first AAV serotype into the capsid protein of a second AAV serotype that is different from said first AAV serotype.

The present invention also provides an AAV capsid comprising the AAV capsid protein of this invention. Further provided herein is a viral vector comprising the AAV capsid of this invention as well as a composition comprising the AAV capsid protein, AAV capsid and/or viral vector of this invention in a pharmaceutically acceptable carrier.

The present invention additionally provides a method of introducing a nucleic acid into a cell in the presence of antibodies against the AAV capsid, comprising contacting the cell with the viral vector of this invention. The cell can be in a subject and in some embodiments, the subject can be a human subject.

These and other aspects of the invention are addressed in more detail in the description of the invention set forth below.

### BRIEF DESCRIPTION OF THE DRAWINGS

FIG. 1. Methods for generating AAVe strains through structural determination of common antigenic motifs (CAMs) listed in Table 5 and the generation of antibody evading AAV capsids (AAVe) by rational or combinatorial engineering of antigenic motifs followed by amplification and selection.

FIG. 2. Generation of AAVe libraries by random mutagenesis of amino acid residues within common antigenic motifs (CAMs) listed in Table 5. Theoretical diversities of different libraries generated by randomizing the different amino acid residues within each common antigenic motif. Successful generation of AAV1e libraries was confirmed via DNA sequencing of the AAV1e plasmids (SEQ ID NOS:439-442). Black solid bar represents the position of the randomized sequences of different AAV1e libraries. Theoretical diversities were calculated by the following equation: Theoretical diversities=20\hat{ }n, where n is the number of randomized amino acids within the indicated CAM. FIG. 2 discloses SEQ ID NO:494.

FIG. 3. In vitro antibody neutralization assay of AAV1e-series. Transduction efficiency was measured by luciferase activity. AAV1 (far left) is neutralized by both 4E4 (top) and 5H7 (bottom) and the 50% inhibition concentration of the two antibodies are <1:64000 and 1:16000 respectively. 4E4 and 5H7 are antibodies that neutralize parental AAV1. Clone AAV1e6 (middle left) is completely resistant to 4E4 neutralization (no reduction in transduction level at the highest antibody concentration) and partially resistant to 5H7 (50% inhibition concentration reduced to 1:4000). Clone AAV1e8 (middle right) showed complete resistance to both 4E4 and 5H7 neutralization where the highest antibody concentration showed no effect on the transduction level. AAV (far right) showed resistance to 5H7; however, it is as sensitive to 4E4 as AAV1.

FIG. 4. In vivo antibody neutralization assay of AAV1e-series at 4 weeks post-injection into skeletal muscle of mice. Representative images of each virus and treatment group are shown. All viruses showed a similar level of transduction efficiency without antibody addition. AAV and AAV show resistance to 4E4 and AAV shows resistance to 5H7. AAV also shows partial resistance to 5H7. 4E4 and 5H7 are antibodies that completely neutralize parental AAV1. Luciferase activities were quantified and summarized in the bar graph (AAV1 is far left; AAV is middle left; AAV is middle right; AAV is far right). These results confirm that the AAV1e series can escape subsets of neutralizing antibodies. Other AAV strains can be subjected to this engineering and selection protocol and similar AAVe vector series can be generated from any capsid template using this approach.

FIG. 5. In vitro antibody neutralization assay of AAV 1e clones derived by rational combination of amino acid residues obtained from AAV1e6, AAv1e8 and AAV1e9. Transduction efficiency was measured by luciferase activity. AAV1 (far left) is completely neutralized by both 4E4 (top) and 5H7 (bottom) as well as the human serum sample containing polyclonal antibodies. The 50% inhibition dilution of human serum sample >1:800 fold dilution. 4E4 and 5H7 are antibodies that neutralize parental AAV1. Clone AAV1e18 (middle left) is partially resistant to 4E4, 5H7 as well as human serum. Clones AAV1e19 and AAV1e20 (middle and far right) showed complete resistance to both 4E4 and 5H7 neutralization as well as the human serum sample.

FIG. 6. Native dot blot assay comparing the parental AAV1 and AAV1e clones 27, 28 and 29 derived by rational, site-specific mutagenesis of residues S472R, V473D and N500E within CAM regions listed in Table 5. Assay determines the ability of AAV1e clones to escape antibody detection. ADK1a is a monoclonal antibody that detects parental AAV1 capsids.

FIG. 7. ELISA assay comparing the parental AAV1 and AAV1e clones 27, 28 and 29 derived by rational, site-specific mutagenesis of residues S472R, V473D and N500E within CAM regions listed in Table 5. Assay determines the ability of AAV1e clones to escape antibody detection. ADK1a is a monoclonal antibody that detects parental AAV1 capsids.

FIG. 8. Transduction assay showing ability of AAV1e27 clone to evade neutralization by ADK1a, which is an anti-capsid antibody against parental AAV1.

FIG. 9. Native dot blot assay comparing the parental AAV1 and clones AAV1e30-36 derived by rational, multiple site-specific mutagenesis within the CAM regions outlined in Table 5. Assay determines the ability of AAV1e clones to escape antibody detection. 4E4 and 5H7 are anti-AAV1 capsid antibodies.

FIG. 10. Transduction assay comparing the parental AAV1 and clones AAV1e30-36 derived by rational, multiple site-specific mutagenesis within the CAM regions outlined in Table 5. Assay determines the ability of AAV1e clones to escape antibody detection. 4E4 and 5H7 are monoclonal antibodies against the parental AAV1 capsid and the human serum sample contains polyclonal antibodies against AAV1. Clones AAV1e30-36 completely escape 4E4, while parental AAV1 is neutralized. Clones AAV1e34 and AAV1e35 show substantial ability to escape 5H7, while AAV1e36 displays a partial ability for evading 5H7. Clone AAV1e36 escapes polyclonal antibodies in a human patient serum sample (50% neutralization for parental AAV1 is 1:320 dilution, while AAV is shifted to between 1:40 and 1:80 dilution range.

FIG. 11. Native dot blot assay comparing the parental AAV9 and clones AAV9e1 and AAV9e2 derived by rational, site-specific mutagenesis of residues listed within the CAM regions outlined in Table 5. Assay establishes the ability to engineer another serotype AAV9 to evade antibodies and the ability of AAV9e clones to escape antibody detection. ADK9, HL2368, HL2370 and HL2372 are monoclonal antibodies that detect parental AAV9 capsids.

FIGS. 12A-12D. Roadmap for structure-based evolution of antigenically advanced AAV variants. (A) Three-dimensional model of cryo-reconstructed AAV1 capsid complexed to multiple monoclonal antibodies. The model depicts AAV1 complexed with the Fab regions of 4 different monoclonal antibodies viewed along the 2-fold axis, ADK1a, ADK1b, 4E4, 5H7. (B) Contact residues and common antigenic motifs (CAMs) for four anti-AAV1 antibodies on the capsid generated by RIVEM are shown. Color codes of each antibody are same as above, in addition, overlapping residues between antibodies were colored individually, ADK1a and 4E4, 4E4 and 5H7. (C) Individual antigenic footprints on the AAV1 capsid selected for engineering and AAV library generation. Three different AAV libraries were subjected to five rounds of evolution on vascular endothelial cells co-infected with adenovirus to yield single region AAV-CAM variants. (D) Newly evolved antigenic footprints from each library were then combined and re-engineered through an iterative process, pooled and subjected to a second round of directed evolution for 3 cycles. This approach yields antigenically advanced AAV-CAM variants with new footprints that have not yet emerged in nature.

FIGS. 13A-13H. Analysis of library diversity, directed evolution and enrichment of novel antigenic footprints. Parental and evolved libraries were subjected to high-throughput sequencing using the Illumina MiSeq platform. Following analysis with a custom Perl script, enriched amino acid sequences were plotted in R for both the parental and evolved libraries of (A) region 4, (B) region 5, (C) region 8 and (D) combined regions 5+8. Each bubble represents a distinct capsid amino acid sequence with the area proportional to the number of reads for that variant in the respective library. (E-H) Amino acid sequence representation was calculated for the top ten variants with the highest representation in each library after subjecting to evolution. Percentages represent the number of reads for the variant in the evolved library normalized to the total number of reads containing the antigenic region of interest. “Other” sequences represent all other evolved library amino acid sequences not contained in the top ten hits. FIG. 13A discloses SEQ ID NOs:22, 23, 25, 483 and 527. FIG. 13B discloses SEQ ID NOs:485, 501 and 502. FIG. 13C discloses SEQ ID NOs:309, 486, 510. FIG. 13D discloses SEQ ID NOs:309 and 487. FIG. 13E discloses SEQ ID NOs:22-25, 483 and 495-500. FIG. 13F discloses SEQ ID NOs:485 and 501-509. FIG. 13G discloses SEQ ID NOs: 32, 37, 38 and 510-515. FIG. 13H discloses SEQ ID NOs:309-487 and 516-523.

FIGS. 14A-14I. Neutralization profile of AAV1 and single region CAM variants against mouse monoclonal antibodies (MAbs) in vitro and in vivo. (A-C) Different AAV strains, AAV1, CAM106, CAM108 and CAM109 evaluated against MAbs 4E4, 5H7 and ADK1a at different dilutions of hybridoma media. Relative luciferase transgene expression mediated by different vectors mixed with MAbs was normalized to no antibody controls. Error bars represent standard deviation (n=4). (D) Roadmap images of the 3-fold axis of each CAM mutant showing the location of newly evolved antigenic footprints—CAM106, CAM108 and CAM109. (E-H) Luciferase expression in mouse hind limb muscles injected with a dose of 2×10¹⁰ vg of AAV1, CAM106, CAM108 and CAM109 vectors packaging ssCBA-Luc and mixed with different MAbs. Representative live animal images at 4 wks post-injection are shown in the following subgroups (E) no antibody control, (F) 4E4 (1:500), (G) 5H7 (1:50) and (H) ADK1a (1:5). (I) Quantitation of luciferase activity mediated by different CAM variants relative to parental AAV1. Luciferase activity is expressed as photons/sec/cm2/sr as calculated by Living Image 3.2 software. Error bars represent S.D. (n=3).

FIGS. 15A-15E. Neutralization profiles of AAV1 and CAM variants in pre-immunized mouse antisera. (A) Roadmap images of each antigenically advanced CAM variant showing newly evolved footprints at the 3-fold symmetry axis—CAM117 (regions 4+5), CAM125 (regions 5+8, cyan) and CAM130 (regions 4+5+8). (B-D) Anti-AAV1 mouse serum from three individual animals and (E) control mouse serum were serially diluted in 2-fold increments from 1:50-1:3200 and co-incubated with AAV vectors in vitro. The dotted line represents NAb-mediated inhibition of AAV transduction by 50%. Solid lines represent relative transduction efficiencies of AAV1, CAM117, CAM125 and CAM130 at different dilutions of antisera. Error bars represented S.D. (n=3).

FIGS. 16A-16I. Neutralization profiles of AAV1 and CAM130 in non-human primate antisera. Serum samples collected from three individual rhesus macaques collected at pre-(naïve) and post-immunization (at 4 wks and 9 wks) were serially diluted at 2-fold increments from 1:5-1:320 and co-incubated with AAV vectors in vitro. The dotted line represents NAb-mediated inhibition of AAV transduction by 50%. Solid lines represent relative transduction efficiencies of AAV1 and CAM130 at different dilutions of antisera. Error bars represented S.D. (n=3).

FIGS. 17A and 17B. Neutralization profile of AAV1 and CAM130 against individual primate and human serum samples. AAV1 and CAM130 packaging CBA-Luc (MOI 10,000) were tested against (A) primate and (B) human sera at a 1:5 dilution to reflect clinically relevant exclusion criteria. The dotted line represents NAb-mediated inhibition of AAV transduction by 50%. Solid bars represent relative transduction efficiencies of AAV1 and CAM130. Error bars represented S.D. (n=3).

FIG. 18A-18D. In vivo characterization of the CAM130 variant. Luciferase transgene expression profiles of AAV1 and CAM130 in (A) heart and (C) liver at 2 wks post-intravenous administration of 1×10¹¹ vg/mouse (n=5). Dotted lines show background levels of luciferase activity in mock injection controls. Biodistribution of AAV1 and CAM130 vector genomes in (B) heart and (D) liver. Vector genome copy numbers per cell were calculated and values from mock injection controls were subtracted to obtain final values. Each dot represented a duplicated experiment from a single animal (n=5) and the dash represents the mean value.

FIGS. 19A-19C. Physical and biological properties of CAM variants compared to AAV1. (A) Titers of purified CAM variants produced using the triple plasmid transfection protocol in HEK293 cells (four 150 mm culture dishes). Transduction profile of (B) single CAM variants and (C) combined CAM variants compared to AAV1 on vascular endothelial cells (MB114).

FIG. 20. Sequencing Reads Mapped to Region of Interest. Percentage of sequencing reads mapped to the mutagenized region of interest for unselected and selected libraries CAMS, CAMS, CAM58, and CAM4. Demultiplexed FASTQ files were processed and mapped with a custom Perl script.

FIG. 21. Representation of lead variants in unselected and selected libraries. Percentage representation of amino acid sequences for lead variants in unselected and selected libraries, calculated by dividing the reads containing a sequence of interest by the total reads containing the mutagenized region. FIG. 21 discloses SEQ ID NOs:534-527.

FIG. 22. Transduction of human hepatocarcinoma cells Huh7 by AAV8e mutants. Transduction efficiency of AAV8e mutants AAV8e01, AAV8e04 and AAV8e05 of Huh7 cells was determined and compared to the transduction of Huh7 cells by wild-type AAV8.

FIGS. 23A-23C. Escape of AAV8e mutants from neutralization by mouse monoclonal antibodies against AAV8. The ability of AAV8e mutants to escape neutralization was examined using mAbs HL2381 (A), HL2383 (B) and ADK8 (C).

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, GenBank accession numbers and other references mentioned herein are incorporated by reference herein in their entirety.

The designation of all amino acid positions in the AAV capsid proteins in the description of the invention and the appended claims is with respect to VP1 capsid subunit numbering. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3).

### Definitions

The following terms are used in the description herein and the appended claims:

The singular forms “a,” “an” and “the” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

As used herein, the terms “reduce,” “reduces,” “reduction” and similar terms mean a decrease of at least about 10%, 15%, 20%, 25%, 35%, 50%, 75%, 80%, 85%, 90%, 95%, 97% or more.

As used herein, the terms “enhance,” “enhances,” “enhancement” and similar terms indicate an increase of at least about 10%, 15%, 20%, 25%, 50%, 75%, 100%, 150%, 200%, 300%, 400%, 500% or more.

The term “parvovirus” as used herein encompasses the family Parvoviridae, including autonomously replicating parvoviruses and dependoviruses. The autonomous parvoviruses include members of the genera Protoparvovirus, Erythroparvovirus, Bocaparvirus, and Densovirus subfamily. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers; Cotmore et al. Archives of Virology DOI 10.1007/s00705-013-1914-I).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al., (2004) J. Virology 78:6381-6388; Moris et al., (2004) Virology 33-:375-383; and Table 1).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al., (1983) J. Virology 45:555; Chiorini et al., (1998) J. Virology 71:6823; Chiorini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; Moris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV9 (DiMattia et al., (2012) J. Virol. 86:6947-6958), AAV8 (Nam et al., (2007) J. Virol. 81:12260-12271), AAV6 (Ng et al., (2010) J. Virol. 84:12945-12957), AAV5 (Govindasamy et al., (2013) J. Virol. 87, 11187-11199), AAV4 (Govindasamy et al., (2006) J. Virol. 80:11556-11570), AAV3B (Lerch et al., (2010) Virology 403: 26-36), BPV (Kailasan et al., (2015) J. Virol. 89:2603-2614) and CPV (Xie et al., (1996) J. Mol. Biol. 6:497-520 and Tsao et al., (1991) Science 251: 1456-64).

TABLE 1

| | GenBank |
|---|---|
| AAV | Accession |
| Serotypes/Isolates | Number |
| Clonal Isolates | |
| Avian AAV ATCC | AY186198, |
| VR-865 | AY629583, |
| | NC_004828 |
| Avian AAV strain | NC_006263, |
| DA-1 | AY629583 |
| Bovine AAV | NC_005889, |
| | AY388617 |
| AAV4 | NC_001829 |
| AAV5 | AY18065, |
| | AF085716 |
| Rh34 | AY243001 |
| Rh33 | AY243002 |
| Rh32 | AY243003 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus, from a non-integrated episome, as well as any other form in which the virus may take within the cell.

As used here, “systemic tropism” and “systemic transduction” (and equivalent terms) indicate that the virus capsid or virus vector of the invention exhibits tropism for or transduces, respectively, tissues throughout the body (e.g., brain, lung, skeletal muscle, heart, liver, kidney and/or pancreas). In embodiments of the invention, systemic transduction of muscle tissues (e.g., skeletal muscle, diaphragm and cardiac muscle) is observed. In other embodiments, systemic transduction of skeletal muscle tissues achieved. For example, in particular embodiments, essentially all skeletal muscles throughout the body are transduced (although the efficiency of transduction may vary by muscle type). In particular embodiments, systemic transduction of limb muscles, cardiac muscle and diaphragm muscle is achieved. Optionally, the virus capsid or virus vector is administered via a systemic route (e.g., systemic route such as intravenously, intra-articularly or intra-lymphatically). Alternatively, in other embodiments, the capsid or virus vector is delivered locally (e.g., to the footpad, intramuscularly, intradermally, subcutaneously, topically).

Unless indicated otherwise, “efficient transduction” or “efficient tropism,” or similar terms, can be determined by reference to a suitable control (e.g., at least about 50%, 60%, 70%, 80%, 85%, 90%, 95% or more of the transduction or tropism, respectively, of the control). In particular embodiments, the virus vector efficiently transduces or has efficient tropism for skeletal muscle, cardiac muscle, diaphragm muscle, pancreas (including (3-islet cells), spleen, the gastrointestinal tract (e.g., epithelium and/or smooth muscle), cells of the central nervous system, lung, joint cells, and/or kidney. Suitable controls will depend on a variety of factors including the desired tropism profile. For example, AAV8 and AAV9 are highly efficient in transducing skeletal muscle, cardiac muscle and diaphragm muscle, but have the disadvantage of also transducing liver with high efficiency. Thus, the invention can be practiced to identify viral vectors of the invention that demonstrate the efficient transduction of skeletal, cardiac and/or diaphragm muscle of AAV8 or AAV9, but with a much lower transduction efficiency for liver. Further, because the tropism profile of interest may reflect tropism toward multiple target tissues, it will be appreciated that a suitable vector may represent some tradeoffs. To illustrate, a virus vector of the invention may be less efficient than AAV8 or AAV9 in transducing skeletal muscle, cardiac muscle and/or diaphragm muscle, but because of low level transduction of liver, may nonetheless be very desirable.

Similarly, it can be determined if a virus “does not efficiently transduce” or “does not have efficient tropism” for a target tissue, or similar terms, by reference to a suitable control. In particular embodiments, the virus vector does not efficiently transduce (i.e., has does not have efficient tropism) for liver, kidney, gonads and/or germ cells. In particular embodiments, undesirable transduction of tissue(s) (e.g., liver) is 20% or less, 10% or less, 5% or less, 1% or less, 0.1% or less of the level of transduction of the desired target tissue(s) (e.g., skeletal muscle, diaphragm muscle, cardiac muscle and/or cells of the central nervous system).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

As used herein, by “isolate” or “purify” (or grammatical equivalents) a virus vector, it is meant that the virus vector is at least partially separated from at least some of the other components in the starting material. In representative embodiments an “isolated” or “purified” virus vector is enriched by at least about 10-fold, 100-fold, 1000-fold, 10,000-fold or more as compared with the starting material.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence” and “heterologous nucleic acid” are used interchangeably herein and refer to a sequence that is not naturally occurring in the virus. Generally, the heterologous nucleic acid comprises an open reading frame that encodes a polypeptide or nontranslated RNA of interest (e.g., for delivery to a cell or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleic acid sequences. rAAV vectors generally require only the terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one TR sequence (e.g., AAV TR sequence), optionally two TRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR” may be from any AAV, including but not limited to serotypes 1, 2, 3, 4, 5, 6, 7, 8, 9, 10, 11, 12, 13 or any other AAV now known or later discovered (see, e.g., Table 1). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

As used herein, the term “amino acid” encompasses any naturally occurring amino acid, modified forms thereof, and synthetic amino acids.

Naturally occurring, levorotatory (L-) amino acids are shown in Table 2).

TABLE 2

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) and/or can be an amino acid that is modified by post-translation modification (e.g., acetylation, amidation, formylation, hydroxylation, methylation, phosphorylation or sulfatation).

TABLE 3

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | alle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid as described by Wang et al., Annu Rev Biophys Biomol Struct. 35:225-49 (2006)). These unnatural amino acids can advantageously be used to chemically link molecules of interest to the AAV capsid protein.

### Modified AAV Capsid Proteins and Virus Capsids and Virus Vectors Comprising the Same.

The present invention provides AAV capsid proteins (VP1, VP2 and/or VP3) comprising a modification (e.g., a substitution) in the amino acid sequence and virus capsids and virus vectors comprising the modified AAV capsid protein. The inventors have discovered that modifications of this invention can confer one or more desirable properties to virus vectors comprising the modified AAV capsid protein including without limitation, the ability to evade neutralizing antibodies. Thus, the present invention addresses some of the limitations associated with conventional AAV vectors.

Accordingly, in one aspect, the present invention provides an adeno-associated virus (AAV) capsid protein, comprising one or more amino acid substitutions, wherein the one or more substitutions modify one or more antigenic sites on the AAV capsid protein. The modification of the one or more antigenic sites results in inhibition of binding by an antibody to the one or more antigenic sites and/or inhibition of neutralization of infectivity of a virus particle comprising said AAV capsid protein. The one or more amino acid substitutions can be in one or more antigenic footprints identified by peptide epitope mapping and/or cryo-electron microscopy studies of AAV-antibody complexes containing AAV capsid proteins. In some embodiments, the one or more antigenic sites are common antigenic motifs or CAMs (see, e.g., Table 5). The capsid proteins of this invention are modified to produce an AAV capsid that is present in an AAV virus particle or AAV virus vector that has a phenotype of evading neutralizing antibodies. The AAV virus particle or vector of this invention can also have a phenotype of enhanced or maintained transduction efficiency in addition to the phenotype of evading neutralizing antibodies.

In some embodiments, the one or more substitutions of the one or more antigenic sites can introduce one or more antigenic sites from a capsid protein of a first AAV serotype into the capsid protein of a second AAV serotype that is different from said first AAV serotype.

The AAV capsid protein of this invention can be a capsid protein of an AAV serotype selected from AAV1, AAV2, AAV3, AAV3B, AAV4, AAV5, AAV6, AAV7, AAV8, AAV9, AAV10, AAV11, AAV12, AAVrh.8, AAVrh.10, AAVrh.32.33, bovine AAV, avian AAV or any other AAV now known or later identified.

Several examples of a modified AAV capsid protein of this invention are provided herein. In the following examples, the capsid protein can comprise the specific substitutions described and in some embodiments can comprise fewer or more substitutions than those described. For example in some embodiments, a capsid protein of this invention can comprise at least 1, 2, 3, 4, 5, 6, 7, 8, 9, 10, etc., substitutions.

Furthermore, in the embodiments described herein wherein an amino acid residue is substituted by any amino acid residue other than the amino acid residue present in the wild type or native amino acid sequence, said any other amino acid residue can be any natural or non-natural amino acid residue known in the art (see, e.g., Tables 2 and 3). In some embodiments, the substitution can be a conservative substitution and in some embodiments, the substitution can be a nonconservative substitution.

In some embodiments, the capsid protein of this invention can comprise a substitution at one or more (e.g., 2, 3, 4, 5, 6, or 7) of amino acid residues 262-268 of AAV1 (VP1 numbering; CAM1), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2, 3, 4, 5, 6, 7, 8, 9, or 10) of amino acid residues 370-379 of AAV1 (VP1 numbering; CAM 3), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2, 3, 4, 5, 6, 7, 8 or 9) of amino acid residues 451-459 of AAV1 (VP1 numbering; CAM 4-1), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2) of amino acid residues 472-473 of AAV1 (VP1 numbering; CAM 4-2) or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2, 3, 4, 5, 6, 7, or 8) of amino acid residues 493-500 of AAV1 (VP1 numbering; CAM 5), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2, 3, 4, 5, 6, or 7) of amino acid residues 528-534 of AAV1 (VP1 numbering; CAM 6), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2, 3, 4, 5, or 6) of amino acid residues 547-552 of AAV1 (VP1 numbering; CAM 7), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2, 3, 4, 5, 6, 7, 8, 9 or 10) of amino acid residues 588-597 of AAV1 (VP1 numbering; CAM 8), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2) of amino acid residues 709-710 of AAV1 (VP1 numbering; CAM 9-1), or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In some embodiments, the capsid protein of this invention can comprise a substitution at one or more (e.g., 2, 3, 4, 5, 6 or 7) of amino acid residues 716-722 of AAV1 (VP1 numbering; CAM 9-2), in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV.

In particular embodiments of this invention, an adeno-associated virus (AAV) capsid protein is provided herein, wherein the capsid protein comprises one or more substitution at all positions or in any combination of fewer than all positions, resulting in the amino acid sequence: X¹-X²-X³-X⁴-X⁵-X⁶-X⁷ (SEQ ID NO:18) at the amino acids corresponding to amino acid positions 262 to 268 (VP1 numbering) of the native AAV1 capsid protein, wherein X¹ is any amino acid other than S; wherein X² is any amino acid other than A; wherein X³ is any amino acid other than S; wherein X⁴ is any amino acid other than T; wherein X⁵ is any amino acid other than G; wherein X⁶ is any amino acid other than A; and wherein X⁷ is any amino acid other than S. In embodiments wherein any of X¹ through X⁷ is not substituted, the amino acid residue at the unsubstituted position is the wild type amino acid residue.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:19) at the amino acids corresponding to amino acid positions 370 to 379 (VP1 numbering) of the native AAV1 capsid protein, wherein X¹ is any amino acid other than V; wherein X² is any amino acid other than F; wherein X³ is any amino acid other than M; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹ (SEQ ID NO:20) at the amino acids corresponding to amino acid positions 451 to 459 (VP1 numbering) of the native AAV1 capsid protein, wherein X¹ is any amino acid other than N; wherein X² is any amino acid other than Q; wherein X³ is any amino acid other than S; wherein X⁴ is any amino acid other than G; wherein X⁵ is any amino acid other than S; wherein X⁶ is any amino acid other than A; wherein X⁷ is any amino acid other than Q; X⁸ is any amino acid other than N and X⁹ is any amino acid other than K. In particular embodiments, X⁶-X⁷-X⁸-X⁹ (SEQ ID NO:21) can be: (a) QVRG (SEQ ID NO:22); (b) ERPR (SEQ ID NO:23); (c) GRGG (SEQ ID NO:24); (d) SGGR (SEQ ID NO:25); (e) SERR (SEQ ID NO:26); or (f) LRGG (SEQ ID NO:27).

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:28) at the amino acids corresponding to amino acid positions 493 to 500 (VP1 numbering) of the native AAV1 capsid protein, wherein X¹ is any amino acid other than K; wherein X² is any amino acid other than T; wherein X³ is any amino acid other than D; wherein X⁴ is any amino acid other than N; wherein X⁵ is any amino acid other than N; wherein X⁶ is any amino acid other than N; wherein X⁷ is any amino acid other than S; and X⁸ is any amino acid other than N. In particular embodiments, X¹-X²-X³-X⁴-X⁵-X⁶-X⁷ (SEQ ID NO:29) can be PGGNATR (SEQ ID NO:30).

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:31) at the amino acids corresponding to amino acid positions 588 to 597 (VP1 numbering) of the native AAV1 capsid protein, wherein X¹ is any amino acid other than S; wherein X² is any amino acid other than T; wherein X³ is any amino acid other than D; wherein X⁴ is any amino acid other than P; wherein X⁵ is any amino acid other than A; wherein X⁶ is any amino acid other than T; wherein X⁷ is any amino acid other than G; wherein X⁸ is any amino acid other than D; wherein X⁹ is any amino acid other than V; and wherein X¹⁰ is any amino acid other than H. In particular embodiments, X¹-X²-X³-X⁴-X⁵-X⁶-X⁷-X⁸-X⁹-X¹⁰ (SEQ ID NO:31) can be: (a) TADHDTKGVL (SEQ ID NO:32); (b) VVDPDKKGVL (SEQ ID NO:33); (c) AKDTGPLNVM (SEQ ID NO:34); (d) QTDAKDNGVQ (SEQ ID NO:35); (e) DKDPWLNDVI (SEQ ID NO:36); (f) TRDGSTESVL (SEQ ID NO:37); (g) VIDPDQKGVL (SEQ ID NO:38); or (h) VNDMSNYMVH (SEQ ID NO:39).

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 709 to 710 (VP1 numbering) of the native AAV1 capsid protein, wherein X¹ is any amino acid other than A; and wherein X² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:40) at the amino acids corresponding to amino acid positions 716 to 722 (VP1 numbering) of the native AAV1 capsid protein, wherein X¹ is any amino acid other than D; wherein X² is any amino acid other than N; wherein X³ is any amino acid other than N; wherein X⁴ is any amino acid other than G; wherein X⁵ is any amino acid other than L; wherein X⁶ is any amino acid other than Y; and wherein X′ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶ (SEQ ID NO:41) at the amino acids corresponding to amino acid positions 262 to 267 (VP1 numbering) of the native AAV2 capsid protein, wherein X¹ is any amino acid other than S; wherein X² is any amino acid other than Q; wherein X³ is any amino acid other than S; wherein X⁴ is any amino acid other than G; wherein X⁵ is any amino acid other than A; and wherein X⁶ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:42) at the amino acids corresponding to amino acid positions 369 to 378 (VP1 numbering) of the native AAV2 capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:43) at the amino acids corresponding to amino acid positions 455 to 458 (VP1 numbering) of the native AAV2 capsid protein, wherein X¹ is any amino acid other than T; wherein X² is any amino acid other than T; wherein X³ is any amino acid other than Q; and wherein X⁴ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:44) at the amino acids corresponding to amino acid positions 492 to 498 (VP1 numbering) of the native AAV2 capsid protein, wherein X¹ is any amino acid other than S; wherein X² is any amino acid other than A; wherein X³ is any amino acid other than D; wherein X⁴ is any amino acid other than N; wherein X⁵ is any amino acid other than N; wherein X⁶ is any amino acid other than N; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:45) at the amino acids corresponding to amino acid positions 587 to 596 (VP1 numbering) of the native AAV2 capsid protein, wherein X¹ is any amino acid other than N; wherein X² is any amino acid other than R; wherein Vis any amino acid other than Q; wherein X⁴ is any amino acid other than A; wherein X⁵ is any amino acid other than A; wherein X⁶ is any amino acid other than T; wherein X⁷ is any amino acid other than A; wherein X⁸ is any amino acid other than D; wherein X⁹ is any amino acid other than V; and wherein X¹⁰ is any amino acid other than N.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 708 to 709 (VP1 numbering) of the native AAV12 capsid protein, wherein X¹ is any amino acid other than V; and wherein X² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:46) at the amino acids corresponding to amino acid positions 715 to 721 (VP1 numbering) of the native AAV2 capsid protein, wherein X¹ is any amino acid other than D; wherein X² is any amino acid other than T; wherein X³ is any amino acid other than N; wherein X⁴ is any amino acid other than G; wherein X⁵ is any amino acid other than V; wherein X⁶ is any amino acid other than Y; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶ (SEQ ID NO:47) at the amino acids corresponding to amino acid positions 262 to 267 (VP1 numbering) of the native AAV3 capsid protein, wherein X¹ is any amino acid other than S; wherein X² is any amino acid other than Q; wherein X³ is any amino acid other than S; wherein X⁴ is any amino acid other than G; wherein X⁵ is any amino acid other than A; and wherein X⁶ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:48) at the amino acids corresponding to amino acid positions 369 to 378 (VP1 numbering) of the native AAV3 capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:49) at the amino acids corresponding to amino acid positions 456 to 459 (VP1 numbering) of the native AAV3 capsid protein, wherein X¹ is any amino acid other than T; wherein X² is any amino acid other than N; wherein X³ is any amino acid other than Q; and wherein X⁴ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:50) at the amino acids corresponding to amino acid positions 493 to 499 (VP1 numbering) of the native AAV3 capsid protein, wherein X¹ is any amino acid other than A; wherein X² is any amino acid other than N; wherein X³ is any amino acid other than D; wherein X⁴ is any amino acid other than N; wherein X⁵ is any amino acid other than N; wherein X⁶ is any amino acid other than N; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:57) at the amino acids corresponding to amino acid positions 588 to 597 (VP1 numbering) of the native AAV3 capsid protein, wherein X¹ is any amino acid other than N; wherein X² is any amino acid other than T; wherein X³ is any amino acid other than A; wherein X⁴ is any amino acid other than P; wherein X⁵ is any amino acid other than T; wherein X⁶ is any amino acid other than T; wherein X⁷ is any amino acid other than G; wherein X⁸ is any amino acid other than T; wherein X⁹ is any amino acid other than V; and wherein X¹⁰ is any amino acid other than N.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 709 to 710 (VP1 numbering) of the native AAV3 capsid protein, wherein X¹ is any amino acid other than V; and wherein X² is any amino acid other than N.

An adeno-associated virus (AAV) capsid protein, wherein the capsid protein comprises a substitution at all positions or in any combination of fewer than all positions, resulting in the amino acid sequence: X¹-X²-X³-X⁴-X⁵-X⁶-X⁷ (SEQ ID NO:52) at the amino acids corresponding to amino acid positions 716 to 722 (VP1 numbering) of the native AAV3 capsid protein, wherein X¹ is any amino acid other than D; wherein X² is any amino acid other than T; wherein X³ is any amino acid other than N; wherein X⁴ is any amino acid other than G; wherein X⁵ is any amino acid other than V; wherein X⁶ is any amino acid other than Y; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:53) at the amino acids corresponding to amino acid positions 253 to 260 (VP1 numbering) of the native AAV4 capsid protein, wherein X¹ is any amino acid other than R; wherein X² is any amino acid other than L; wherein X³ is any amino acid other than G; wherein X⁴ is any amino acid other than E; wherein X⁵ is any amino acid other than S; wherein X⁶ is any amino acid other than L; wherein X⁷ is any amino acid other than Q; and wherein X⁸ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:54) at the amino acids corresponding to amino acid positions 360 to 369 (VP1 numbering) of the native AAV4 capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y: and wherein X¹⁰ is any amino acid other than C.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:55) at the amino acids corresponding to amino acid positions 450 to 453 (VP1 numbering) of the native AAV4 capsid protein, wherein X¹ is any amino acid other than A; wherein X² is any amino acid other than G; wherein X³ is any amino acid other than T; and wherein X⁴ is any amino acid other than A.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰-X¹¹-X¹² (SEQ ID NO:56) at the amino acids corresponding to amino acid positions 487 to 498 (VP1 numbering) of the native AAV4 capsid protein, wherein X¹ is any amino acid other than A; wherein X² is any amino acid other than N; wherein X³ is any amino acid other than Q; wherein X⁴ is any amino acid other than N; wherein X⁵ is any amino acid other than Y; wherein X⁶ is any amino acid other than K; wherein X⁷ is any amino acid other than I; wherein X⁸ is any amino acid other than P; wherein X⁹ is any amino acid other than A; wherein X¹⁰ is any amino acid other than T; wherein X¹¹ is any amino acid other than G; and wherein X¹² is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:57) at the amino acids corresponding to amino acid positions 586 to 595 (VP1 numbering) of the native AAV4 capsid protein, wherein X¹ is any amino acid other than S; wherein X² is any amino acid other than N; wherein X³ is any amino acid other than L; wherein X⁴ is any amino acid other than P; wherein X⁵ is any amino acid other than T; wherein X⁶ is any amino acid other than V; wherein X⁷ is any amino acid other than D; wherein X⁸ is any amino acid other than R; wherein X⁹ is any amino acid other than L; and wherein X¹⁰ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 707 to 708 (VP1 numbering) of the native AAV4 capsid protein, wherein X¹ is any amino acid other than N; and wherein X² is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:58) at the amino acids corresponding to amino acid positions 714 to 720 (VP1 numbering) of the native AAV4 capsid protein, wherein X¹ is any amino acid other than D; wherein X² is any amino acid other than A; wherein X³ is any amino acid other than A; wherein X⁴ is any amino acid other than G; wherein X⁵ is any amino acid other than K; wherein X⁶ is any amino acid other than Y; and wherein X⁷ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:59) at the amino acids corresponding to amino acid positions 249 to 258 (VP1 numbering) of the native AAV5 capsid protein, wherein X¹ is any amino acid other than E; wherein X² is any amino acid other than I; wherein X³ is any amino acid other than K; wherein X⁴ is any amino acid other than S; wherein X⁵ is any amino acid other than G; wherein X⁶ is any amino acid other than S; wherein X⁷ is any amino acid other than V; wherein X⁸ is any amino acid other than D; wherein X⁹ is any amino acid other than G; and wherein X¹⁰ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:60) at the amino acids corresponding to amino acid positions 360 to 369 (VP1 numbering) of the native AAV5 capsid protein, wherein X¹ is any amino acid other than V; wherein X² is any amino acid other than F; wherein X³ is any amino acid other than T; wherein X⁴ is any amino acid other than L; wherein X⁵ is any amino acid other than P wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than A.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:61) at the amino acids corresponding to amino acid positions 443 to 446 (VP1 numbering) of the native AAV5 capsid protein, wherein X¹ is any amino acid other than N; wherein X² is any amino acid other than T; wherein X³ is any amino acid other than G; and wherein X⁴ is any amino acid other than G.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:62) at the amino acids corresponding to amino acid positions 479 to 485 (VP1 numbering) of the native AAV5 capsid protein, wherein X¹ is any amino acid other than S; wherein X² is any amino acid other than G; wherein X³ is any amino acid other than V; wherein X⁴ is any amino acid other than N wherein X⁵ is any amino acid other than R; wherein X⁶ is any amino acid other than A; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:63) at the amino acids corresponding to amino acid positions 577 to 586 (VP1 numbering) of the native AAV5 capsid protein, wherein X¹ is any amino acid other than T; wherein X² is any amino acid other than T; wherein X³ is any amino acid other than A; wherein X⁴ is any amino acid other than P; wherein X⁵ is any amino acid other than A; wherein X⁶ is any amino acid other than T; wherein X⁷ is any amino acid other than G; wherein X⁸ is any amino acid other than T; wherein X⁹ is any amino acid other than Y; and wherein X¹⁰ is any amino acid other than N.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 697 to 698 (VP1 numbering) of the native AAV5 capsid protein, wherein X¹ is any amino acid other than Q; and wherein X² is any amino acid other than F.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:64) at the amino acids corresponding to amino acid positions 704 to 710 (VP1 numbering) of the native AAV5 capsid protein, wherein X¹ is any amino acid other than D; wherein X² is any amino acid other than S; wherein X³ is any amino acid other than T; wherein X⁴ is any amino acid other than G; wherein X⁵ is any amino acid other than E; wherein X⁶ is any amino acid other than Y; and wherein X⁷ is any amino acid other than R.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:65) at the amino acids corresponding to amino acid positions 262 to 268 (VP1 numbering) of the native AAV6 capsid protein, wherein X¹ is any amino acid other than S; wherein X² is any amino acid other than A; wherein X³ is any amino acid other than S; wherein X⁴ is any amino acid other than T; wherein X⁵ is any amino acid other than G; wherein X⁶ is any amino acid other than A; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:66) at the amino acids corresponding to amino acid positions 370 to 379 (VP1 numbering) of the native AAV6 capsid protein, wherein X¹ is any amino acid other than V; wherein X² is any amino acid other than F; wherein X³ is any amino acid other than M; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:67) at the amino acids corresponding to amino acid positions 456 to 459 (VP1 numbering) of the native AAV6 capsid protein, wherein X¹ is any amino acid other than A; wherein X² is any amino acid other than Q; wherein X³ is any amino acid other than N; and wherein X⁴ is any amino acid other than K.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:68) at the amino acids corresponding to amino acid positions 493 to 499 (VP1 numbering) of the native AAV6 capsid protein, wherein X¹ is any amino acid other than K; wherein X² is any amino acid other than T; wherein X³ is any amino acid other than D; wherein X⁴ is any amino acid other than N; wherein X⁵ is any amino acid other than N; wherein X⁶ is any amino acid other than N; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:69) at the amino acids corresponding to amino acid positions 588 to 597 (VP1 numbering) of the native AAV6 capsid protein, wherein X¹ is any amino acid other than S; wherein X² is any amino acid other than T; wherein X³ is any amino acid other than D; wherein X⁴ is any amino acid other than P; wherein X⁵ is any amino acid other than A; wherein X⁶ is any amino acid other than T; wherein X⁷ is any amino acid other than G; wherein X⁸ is any amino acid other than D; wherein X⁹ is any amino acid other than V; and wherein X¹⁰ is any amino acid other than H.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 709 to 710 (VP1 numbering) of the native AAV6 capsid protein, wherein X¹ is any amino acid other than A; and wherein X² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:70) at the amino acids corresponding to amino acid positions 716 to 722 (VP1 numbering) of the native AAV6 capsid protein, wherein X¹ is any amino acid other than D; wherein X² is any amino acid other than N; wherein X³ is any amino acid other than N; wherein X⁴ is any amino acid other than G; wherein X⁵ is any amino acid other than L; wherein X⁶ is any amino acid other than Y; and wherein X⁷ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:71) at the amino acids corresponding to amino acid positions 263 to 269 (VP1 numbering) of the native AAV7 capsid protein, wherein X¹ is any amino acid other than S; wherein X² is any amino acid other than E; wherein X³ is any amino acid other than T; wherein X⁴ is any amino acid other than A; wherein X⁵ is any amino acid other than G; wherein X⁶ is any amino acid other than S; and wherein X⁷ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:72) at the amino acids corresponding to amino acid positions 371 to 380 (VP1 numbering) of the native AAV7 capsid protein, wherein X¹ is any amino acid other than V; wherein X² is any amino acid other than F; wherein X³ is any amino acid other than M; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:73) at the amino acids corresponding to amino acid positions 458 to 461 (VP1 numbering) of the native AAV7 capsid protein, wherein X¹ is any amino acid other than A; wherein X² is any amino acid other than G; wherein X³ is any amino acid other than N; and wherein X⁴ is any amino acid other than R.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:74) at the amino acids corresponding to amino acid positions 495 to 501 (VP1 numbering) of the native AAV7 capsid protein, wherein X¹ is any amino acid other than L; wherein X² is any amino acid other than D; wherein X³ is any amino acid other than Q; wherein X⁴ is any amino acid other than N; wherein X⁵ is any amino acid other than N; wherein X⁶ is any amino acid other than N; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:75) at the amino acids corresponding to amino acid positions 589 to 598 (VP1 numbering) of the native AAV7 capsid protein, wherein X¹ is any amino acid other than N; wherein X² is any amino acid other than T; wherein X³ is any amino acid other than A; wherein X⁴ is any amino acid other than A; wherein X⁵ is any amino acid other than Q; wherein X⁶ is any amino acid other than T; wherein X⁷ is any amino acid other than Q; wherein X⁸ is any amino acid other than V; wherein X⁹ is any amino acid other than V; and wherein X¹⁰ is any amino acid other than N.

An adeno-associated virus (AAV) capsid protein is also provided herein, wherein the capsid protein comprises a substitution resulting in the amino acid sequence: X¹-X² at the amino acids corresponding to amino acid positions 710 to 711 (VP1 numbering) of the native AAV7 capsid protein, wherein X¹ is any amino acid other than T; and wherein X² is any amino acid other than G;

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:76) at the amino acids corresponding to amino acid positions 717 to 723 (VP1 numbering) of the native AAV7 capsid protein, wherein X¹ is any amino acid other than D; wherein X² is any amino acid other than S; wherein X³ is any amino acid other than Q; wherein X⁴ is any amino acid other than G; wherein X⁵ is any amino acid other than V; wherein X⁶ is any amino acid other than Y; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:77) at the amino acids corresponding to amino acid positions 263 to 270 (VP1 numbering) of the native AAV8 capsid protein, wherein X¹ is any amino acid other than N; wherein X² is any amino acid other than G; wherein X³ is any amino acid other than T; wherein X⁴ is any amino acid other than S; wherein X⁵ is any amino acid other than G; wherein X⁶ is any amino acid other than G; wherein X⁷ is any amino acid other than A; and wherein X⁸ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:78) at the amino acids corresponding to amino acid positions 372 to 381 (VP1 numbering) of the native AAV8 capsid protein, wherein X¹ is any amino acid other than V; wherein X² is any amino acid other than F; wherein X³ is any amino acid other than M; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:79) at the amino acids corresponding to amino acid positions 458 to 461 (VP1 numbering) of the native AAV8 capsid protein, wherein X¹ is any amino acid other than A; wherein X² is any amino acid other than N; wherein X³ is any amino acid other than T; and wherein X⁴ is any amino acid other than Q.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:80) at the amino acids corresponding to amino acid positions 495 to 501 (VP1 numbering) of the native AAV8 capsid protein, wherein X¹ is any amino acid other than T; wherein X² is any amino acid other than G; wherein X³ is any amino acid other than Q; wherein X⁴ is any amino acid other than N wherein X⁵ is any amino acid other than N; wherein X⁶ is any amino acid other than N; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁵-X⁶-X⁷-X⁸-X⁹-X¹⁰ (SEQ ID NO:81) at the amino acids corresponding to amino acid positions 590 to 600 (VP1 numbering) of the native AAV8 capsid protein, wherein X¹ is any amino acid other than N; wherein X² is any amino acid other than T; wherein X³ is any amino acid other than A; wherein X⁴ is any amino acid other than P; wherein X⁵ is any amino acid other than Q; wherein X⁶ is any amino acid other than I; wherein Vis any amino acid other than G; wherein X⁸ is any amino acid other than T; wherein X⁹ is any amino acid other than V; wherein X¹⁰ is any amino acid other than N; and wherein X¹¹ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 711 to 712 (VP1 numbering) of the native AAV8 capsid protein, wherein X¹ is any amino acid other than T; and wherein X² is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:82) at the amino acids corresponding to amino acid positions 718 to 724 (VP1 numbering) of the native AAV8 capsid protein, wherein X¹ is any amino acid other than N; wherein X² is any amino acid other than T; wherein X³ is any amino acid other than E; wherein X⁴ is any amino acid other than G; wherein X⁵ is any amino acid other than V; wherein X⁶ is any amino acid other than Y; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:83) at the amino acids corresponding to amino acid positions 262 to 269 (VP1 numbering) of the native AAV9 capsid protein, wherein X¹ is any amino acid other than N; wherein X² is any amino acid other than S; wherein X³ is any amino acid other than T; wherein X⁴ is any amino acid other than S; wherein X⁵ is any amino acid other than G; wherein X⁶ is any amino acid other than G; wherein X⁷ is any amino acid other than S; and wherein X⁸ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:84) at the amino acids corresponding to amino acid positions 371 to 380 (VP1 numbering) of the native AAV9 capsid protein, wherein X¹ is any amino acid other than V; wherein X² is any amino acid other than F; wherein X³ is any amino acid other than M; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:85) at the amino acids corresponding to amino acid positions 456 to 459 (VP1 numbering) of the native AAV9 capsid protein, wherein X¹ is any amino acid other than Q; wherein X² is any amino acid other than N; wherein X³ is any amino acid other than Q; and wherein X⁴ is any amino acid other than Q.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:86) at the amino acids corresponding to amino acid positions 493 to 499 (VP1 numbering) of the native AAV9 capsid protein, wherein X¹ is any amino acid other than V; wherein X² is any amino acid other than T; wherein X³ is any amino acid other than Q; wherein X⁴ is any amino acid other than N; wherein X⁵ is any amino acid other than N; wherein X⁶ is any amino acid other than N; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁵-X⁶-X⁷-X⁸-X⁹-X¹⁰ (SEQ ID NO:87) at the amino acids corresponding to amino acid positions 588 to 597 (VP1 numbering) of the native AAV9 capsid protein, wherein X¹ is any amino acid other than Q; wherein X² is any amino acid other than A; wherein X³ is any amino acid other than Q; wherein X⁴ is any amino acid other than A; wherein X⁵ is any amino acid other than Q; wherein X⁶ is any amino acid other than T; wherein X⁷ is any amino acid other than G; wherein X⁸ is any amino acid other than W; wherein X⁹ is any amino acid other than V; and

wherein X¹⁰ is any amino acid other than Q.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 709 to 710 (VP1 numbering) of the native AAV9 capsid protein, wherein X¹ is any amino acid other than N; and wherein X² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:88) at the amino acids corresponding to amino acid positions 716 to 722 (VP1 numbering) of the native AAV9 capsid protein, wherein X¹ is any amino acid other than N; wherein X² is any amino acid other than T; wherein X³ is any amino acid other than E; wherein X⁴ is any amino acid other than G; wherein X⁵ is any amino acid other than V; wherein X⁶ is any amino acid other than Y; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:89) at the amino acids corresponding to amino acid positions 263 to 270 (VP1 numbering) of the native AAVrh10 capsid protein, wherein X¹ is any amino acid other than N; wherein X² is any amino acid other than G; wherein X³ is any amino acid other than T; wherein X⁴ is any amino acid other than S; wherein X⁵ is any amino acid other than G; wherein X⁶ is any amino acid other than G; wherein X⁷ is any amino acid other than S; and wherein X⁸ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:90) at the amino acids corresponding to amino acid positions 372 to 381 (VP1 numbering) of the native AAVrh10 capsid protein, wherein X¹ is any amino acid other than V; wherein X² is any amino acid other than F; wherein X³ is any amino acid other than M; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:91) at the amino acids corresponding to amino acid positions 458 to 461 (VP1 numbering) of the native AAVrh10 capsid protein, wherein X¹ is any amino acid other than A; wherein X² is any amino acid other than G; wherein X³ is any amino acid other than T; and wherein X⁴ is any amino acid other than Q.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:92) at the amino acids corresponding to amino acid positions 495 to 501 (VP1 numbering) of the native AAVrh10 capsid protein, wherein X¹ is any amino acid other than L; wherein X² is any amino acid other than S; wherein X³ is any amino acid other than Q; wherein X⁴ is any amino acid other than N; wherein X⁵ is any amino acid other than N; wherein X⁶ is any amino acid other than N; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:93) at the amino acids corresponding to amino acid positions 590 to 599 (VP1 numbering) of the native AAVrh10 capsid protein, wherein X¹ is any amino acid other than N; wherein X² is any amino acid other than A; wherein X³ is any amino acid other than A; wherein X⁴ is any amino acid other than P; wherein X⁵ is any amino acid other than I; wherein X⁶ is any amino acid other than V; wherein X⁷ is any amino acid other than G; wherein X⁸ is any amino acid other than A; wherein X⁹ is any amino acid other than V; and wherein X¹⁰ is any amino acid other than N.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 711 to 712 (VP1 numbering) of the native AAVrh10 capsid protein, wherein X¹ is any amino acid other than T; and wherein X² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:94) at the amino acids corresponding to amino acid positions 718 to 724 (VP1 numbering) of the native AAVrh10 capsid protein, wherein X¹ is any amino acid other than N; wherein X² is any amino acid other than T; wherein X³ is any amino acid other than D; wherein X⁴ is any amino acid other than G; wherein X⁵ is any amino acid other than T; wherein X⁶ is any amino acid other than Y; and wherein X⁷ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:95) at the amino acids corresponding to amino acid positions 262 to 269 (VP1 numbering) of the native AAVrh8 capsid protein, wherein X¹ is any amino acid other than N; wherein X² is any amino acid other than G; wherein X³ is any amino acid other than T; wherein X⁴ is any amino acid other than S; wherein X⁵ is any amino acid other than G; wherein X⁶ is any amino acid other than G; wherein X⁷ is any amino acid other than S; and wherein X⁸ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:96) at the amino acids corresponding to amino acid positions 371 to 380 (VP1 numbering) of the native AAVrh8 capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:97) at the amino acids corresponding to amino acid positions 456 to 459 (VP1 numbering) of the native AAVrh8 capsid protein, wherein X¹ is any amino acid other than G; wherein X² is any amino acid other than G; wherein X³ is any amino acid other than T; and wherein X⁴ is any amino acid other than Q.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:98) at the amino acids corresponding to amino acid positions 493 to 499 (VP1 numbering) of the native AAVrh8 capsid protein, wherein X¹ is any amino acid other than T; wherein X² is any amino acid other than N; wherein X³ is any amino acid other than Q; wherein X⁴ is any amino acid other than N; wherein X⁵ is any amino acid other than N; wherein X⁶ is any amino acid other than N; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:99) at the amino acids corresponding to amino acid positions 588 to 597 (VP1 numbering) of the native AAVrh8 capsid protein, wherein X¹ is any amino acid other than N; wherein X² is any amino acid other than T; wherein X³ is any amino acid other than Q; wherein X⁴ is any amino acid other than A; wherein X⁵ is any amino acid other than Q; wherein X⁶ is any amino acid other than T; wherein X⁷ is any amino acid other than G; wherein X⁸ is any amino acid other than L; wherein X⁹ is any amino acid other than V; and wherein X¹⁰ is any amino acid other than H.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 709 to 710 (VP1 numbering) of the native AAVrh8 capsid protein, wherein X¹ is any amino acid other than T; and wherein X² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:100) at the amino acids corresponding to amino acid positions 716 to 722 (VP1 numbering) of the native AAVrh8 capsid protein, wherein X¹ is any amino acid other than N; wherein X² is any amino acid other than T; wherein X³ is any amino acid other than E; wherein X⁴ is any amino acid other than G; wherein X⁵ is any amino acid other than V; wherein X⁶ is any amino acid other than Y; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:101) at the amino acids corresponding to amino acid positions 263 to 270 (VP1 numbering) of the native AAV10 capsid protein, wherein X¹ is any amino acid other than N; wherein X² is any amino acid other than G; wherein X³ is any amino acid other than T; wherein X⁴ is any amino acid other than S; wherein X⁵ is any amino acid other than G; wherein X⁶ is any amino acid other than G; wherein X⁷ is any amino acid other than S; and wherein X⁸ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:102) at the amino acids corresponding to amino acid positions 372 to 381 (VP1 numbering) of the native AAV10 capsid protein, wherein X¹ is any amino acid other than V; wherein X² is any amino acid other than F; wherein X³ is any amino acid other than M; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than L.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:103) at the amino acids corresponding to amino acid positions 458 to 461 (VP1 numbering) of the native AAV10 capsid protein, wherein X¹ is any amino acid other than Q; wherein X² is any amino acid other than G; wherein X³ is any amino acid other than T; and wherein X⁴ is any amino acid other than Q.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:104) at the amino acids corresponding to amino acid positions 495 to 501 (VP1 numbering) of the native AAV10 capsid protein, wherein X¹ is any amino acid other than L; wherein X² is any amino acid other than S; wherein X³ is any amino acid other than Q; wherein X⁴ is any amino acid other than N; wherein X⁵ is any amino acid other than N; wherein X⁶ is any amino acid other than N; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:105) at the amino acids corresponding to amino acid positions 590 to 599 (VP1 numbering) of the native AAV10 capsid protein, wherein X¹ is any amino acid other than N; wherein X² is any amino acid other than T; wherein X³ is any amino acid other than G; wherein X⁴ is any amino acid other than P; wherein X⁵ is any amino acid other than I; wherein X⁶ is any amino acid other than V; wherein X⁷ is any amino acid other than G; wherein X⁸ is any amino acid other than N; wherein X⁹ is any amino acid other than V; and wherein X¹⁰ is any amino acid other than N.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 711 to 712 (VP1 numbering) of the native AAV10 capsid protein, wherein X¹ is any amino acid other than T; and wherein X² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:106) at the amino acids corresponding to amino acid positions 718 to 724 (VP1 numbering) of the native AAV10 capsid protein, wherein X¹ is any amino acid other than N; wherein X² is any amino acid other than T; wherein X³ is any amino acid other than E; wherein X⁴ is any amino acid other than G; wherein X⁵ is any amino acid other than T; wherein X⁶ is any amino acid other than Y; and wherein Vis any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:107) at the amino acids corresponding to amino acid positions 253 to 260 (VP1 numbering) of the native AAV11 capsid protein, wherein X¹ is any amino acid other than R; wherein X² is any amino acid other than L; wherein X³ is any amino acid other than G; wherein X⁴ is any amino acid other than T; wherein X⁵ is any amino acid other than T; wherein X⁶ is any amino acid other than S; wherein X⁷ is any amino acid other than S; and wherein X⁸ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:108) at the amino acids corresponding to amino acid positions 360 to 369 (VP1 numbering) of the native AAV11 capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than C.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:109) at the amino acids corresponding to amino acid positions 449 to 452 (VP1 numbering) of the native AAV11 capsid protein, wherein X¹ is any amino acid other than Q; wherein X² is any amino acid other than G; wherein X³ is any amino acid other than N; and wherein X⁴ is any amino acid other than A.

An adeno-associated virus (AAV) capsid protein is also provided herein, wherein the capsid protein comprises a substitution at all positions or in any combination of fewer than all positions, resulting in the amino acid sequence: X¹-X²-X³-X⁵-X⁶-X⁷-X⁸-X⁹-X¹⁰-X¹¹-X¹² (SEQ ID NO:110) at the amino acids corresponding to amino acid positions 486 to 497 (VP1 numbering) of the native AAV11 capsid protein, wherein X¹ is any amino acid other than A; wherein X² is any amino acid other than S; wherein X³ is any amino acid other than Q; wherein X⁴ is any amino acid other than N; wherein X⁵ is any amino acid other than Y; wherein X⁶ is any amino acid other than K; wherein X⁷ is any amino acid other than I; wherein X⁸ is any amino acid other than P; wherein X⁹ is any amino acid other than A; wherein X¹⁰ is any amino acid other than S; wherein X¹¹ is any amino acid other than G; and wherein X¹² is any amino acid other than G.

An adeno-associated virus (AAV) capsid protein is also provided herein, wherein the capsid protein comprises a substitution at all positions or in any combination of fewer than all positions, resulting in the amino acid sequence: X¹-X²-X³-X⁵-X⁶-X⁷-X⁸-X⁹-X¹⁰ (SEQ ID NO:111) at the amino acids corresponding to amino acid positions 585 to 594 (VP1 numbering) of the native AAV11 capsid protein, wherein X¹ is any amino acid other than T; wherein X² is any amino acid other than T; wherein X³ is any amino acid other than A; wherein X⁴ is any amino acid other than P; wherein X⁵ is any amino acid other than I; wherein X⁶ is any amino acid other than T; wherein X⁷ is any amino acid other than G; wherein X⁸ is any amino acid other than N; wherein X⁹ is any amino acid other than V; and wherein X¹⁰ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 706 to 707 (VP1 numbering) of the native AAV11 capsid protein, wherein X¹ is any amino acid other than S; and wherein X² is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:112) at the amino acids corresponding to amino acid positions 713 to 719 (VP1 numbering) of the native AAV11 capsid protein, wherein X¹ is any amino acid other than D; wherein X² is any amino acid other than T; wherein X³ is any amino acid other than T; wherein X⁴ is any amino acid other than G; wherein X⁵ is any amino acid other than K; wherein X⁶ is any amino acid other than Y; and wherein X⁷ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:113) at the amino acids corresponding to amino acid positions 262 to 269 (VP1 numbering) of the native AAV12 capsid protein, wherein X¹ is any amino acid other than R; wherein X² is any amino acid other than I; wherein X³ is any amino acid other than G; wherein X⁴ is any amino acid other than T; wherein X⁵ is any amino acid other than T; wherein X⁶ is any amino acid other than A; wherein X⁷ is any amino acid other than N; and wherein X⁸ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:114) at the amino acids corresponding to amino acid positions 369 to 378 (VP1 numbering) of the native AAV12 capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than C.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:115) at the amino acids corresponding to amino acid positions 458 to 461 (VP1 numbering) of the native AAV12 capsid protein, wherein X¹ is any amino acid other than Q; wherein X² is any amino acid other than G; wherein X³ is any amino acid other than T; and wherein X⁴ is any amino acid other than A.

An adeno-associated virus (AAV) capsid protein is also provided herein, wherein the capsid protein comprises a substitution at all positions or in any combination of fewer than all positions, resulting in the amino acid sequence: X¹-X²-X³-X⁵-X⁶-X⁷-X⁸-X⁹-X¹⁰-X¹¹-X¹² (SEQ ID NO:116) at the amino acids corresponding to amino acid positions 495 to 506 (VP1 numbering) of the native AAV12 capsid protein, wherein X¹ is any amino acid other than A; wherein X² is any amino acid other than N; wherein X³ is any amino acid other than Q; wherein X⁴ is any amino acid other than N; wherein X⁵ is any amino acid other than Y; wherein X⁶ is any amino acid other than K; wherein X⁷ is any amino acid other than I; wherein X⁸ is any amino acid other than P; wherein X⁹ is any amino acid other than A; wherein X¹⁰ is any amino acid other than S; wherein X¹¹ is any amino acid other than G; and wherein X¹² is any amino acid other than G.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:117) at the amino acids corresponding to amino acid positions 594 to 601 (VP1 numbering) of the native AAV12 capsid protein, wherein X¹ is any amino acid other than T; wherein X² is any amino acid other than T; wherein X³ is any amino acid other than A; wherein X⁴ is any amino acid other than P; wherein X⁵ is any amino acid other than H; wherein X⁶ is any amino acid other than I; wherein X⁷ is any amino acid other than A; wherein X⁸ is any amino acid other than N; wherein X⁹ is any amino acid other than L; and wherein X¹⁰ is any amino acid other than D.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 715 to 716 (VP1 numbering) of the native AAV12 capsid protein, wherein X¹ is any amino acid other than N; and wherein X² is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:118) at the amino acids corresponding to amino acid positions 722 to 728 (VP1 numbering) of the native AAV12 capsid protein, wherein X¹ is any amino acid other than D; wherein X² is any amino acid other than N; wherein X³ is any amino acid other than A; wherein X⁴ is any amino acid other than G; wherein X⁵ is any amino acid other than N; wherein X⁶ is any amino acid other than Y; and wherein X⁷ is any amino acid other than H.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ SEQ ID NO:119) at the amino acids corresponding to amino acid positions 253 to 260 (VP1 numbering) of the native AAVrh32.33 capsid protein, wherein X¹ is any amino acid other than R; wherein X² is any amino acid other than L; wherein X³ is any amino acid other than G; wherein X⁴ is any amino acid other than T; wherein X⁵ is any amino acid other than T; wherein X⁶ is any amino acid other than S; wherein X⁷ is any amino acid other than N; and wherein X⁸ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:120) at the amino acids corresponding to amino acid positions 360 to 369 (VP1 numbering) of the native AAVrh32.33 capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than C.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:121) at the amino acids corresponding to amino acid positions 449 to 452 (VP1 numbering) of the native AAVrh32.33 capsid protein, wherein X¹ is any amino acid other than Q; wherein X² is any amino acid other than G; wherein X³ is any amino acid other than N; and wherein X⁴ is any amino acid other than A.

An adeno-associated virus (AAV) capsid protein is also provided herein, wherein the capsid protein comprises a substitution at all positions or in any combination of fewer than all positions, resulting in the amino acid sequence: X¹-X²-X³-X⁵-X⁶-X⁷-X⁸-X⁹-X¹⁰-X¹¹-X¹² (SEQ ID NO:122) at the amino acids corresponding to amino acid positions 486 to 497 (VP1 numbering) of the native AAVrh32.33 capsid protein, wherein X¹ is any amino acid other than A; wherein X² is any amino acid other than S; wherein X³ is any amino acid other than Q; wherein X⁴ is any amino acid other than N; wherein X⁵ is any amino acid other than Y; wherein X⁶ is any amino acid other than K; wherein X⁷ is any amino acid other than I; wherein X⁸ is any amino acid other than P; wherein X⁹ is any amino acid other than A; wherein X¹⁰ is any amino acid other than S; wherein X¹¹ is any amino acid other than G; and wherein X¹² is any amino acid other than G.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:123) at the amino acids corresponding to amino acid positions 585 to 594 (VP1 numbering) of the native AAVrh32.33 capsid protein, wherein X¹ is any amino acid other than T; wherein X² is any amino acid other than T; wherein X³ is any amino acid other than A; wherein X⁴ is any amino acid other than P; wherein X⁵ is any amino acid other than I; wherein X⁶ is any amino acid other than T; wherein X⁷ is any amino acid other than G; wherein X⁸ is any amino acid other than N; wherein X⁹ is any amino acid other than V; and wherein X¹⁰ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 706 to 707 (VP1 numbering) of the native AAVrh32.33 capsid protein, wherein X¹ is any amino acid other than S; and wherein X² is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:124) at the amino acids corresponding to amino acid positions 713 to 719 (VP1 numbering) of the native AAVrh32.33 capsid protein, wherein X¹ is any amino acid other than D; wherein X² is any amino acid other than T; wherein X³ is any amino acid other than T; wherein X⁴ is any amino acid other than G; wherein X⁵ is any amino acid other than K; wherein X⁶ is any amino acid other than Y; and wherein X⁷ is any amino acid other than T.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:125) at the amino acids corresponding to amino acid positions 255 to 262 (VP1 numbering) of the native bovine AAV capsid protein, wherein X¹ is any amino acid other than R; wherein X² is any amino acid other than L; wherein X³ is any amino acid other than G; wherein X⁴ is any amino acid other than S; wherein X⁵ is any amino acid other than S; wherein X⁶ is any amino acid other than N; wherein X⁷ is any amino acid other than A; and wherein X⁸ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:126) at the amino acids corresponding to amino acid positions 362 to 371 (VP1 numbering) of the native bovine AAV capsid protein, wherein X¹ is any amino acid other than V; wherein X² is any amino acid other than F; wherein X³ is any amino acid other than M; wherein X⁴ is any amino acid other than V; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than C.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:127) at the amino acids corresponding to amino acid positions 452 to 455 (VP1 numbering) of the native bovine AAV capsid protein, wherein X¹ is any amino acid other than Q; wherein X² is any amino acid other than G; wherein X³ is any amino acid other than N; and wherein X⁴ is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰-X¹¹-X¹² (SEQ ID NO:128) at the amino acids corresponding to amino acid positions 489 to 500 (VP1 numbering) of the native bovine AAV capsid protein, wherein X¹ is any amino acid other than A; wherein X² is any amino acid other than S; wherein X³ is any amino acid other than Q; wherein X⁴ is any amino acid other than N; wherein X⁵ is any amino acid other than Y; wherein X⁶ is any amino acid other than K; wherein X⁷ is any amino acid other than I; wherein X⁸ is any amino acid other than P; wherein X⁹ is any amino acid other than Q; wherein X¹⁰ is any amino acid other than G; wherein X¹¹ is any amino acid other than R; and wherein X¹² is any amino acid other than N.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:129) at the amino acids corresponding to amino acid positions 588 to 597 (VP1 numbering) of the native bovine AAV capsid protein, wherein X¹ is any amino acid other than T; wherein X² is any amino acid other than T; wherein X³ is any amino acid other than V; wherein X⁴ is any amino acid other than P; wherein X⁵ is any amino acid other than T; wherein X⁶ is any amino acid other than V; wherein X⁷ is any amino acid other than D; wherein X⁸ is any amino acid other than D; wherein X⁹ is any amino acid other than V; and wherein X¹⁰ is any amino acid other than D.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 709 to 710 (VP1 numbering) of the native bovine AAV capsid protein, wherein X¹ is any amino acid other than D; and wherein X² is any amino acid other than S.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:130) at the amino acids corresponding to amino acid positions 716 to 722 (VP1 numbering) of the native bovine AAV capsid protein, wherein X¹ is any amino acid other than D; wherein X² is any amino acid other than N; wherein X³ is any amino acid other than A; wherein X⁴ is any amino acid other than G; wherein X⁵ is any amino acid other than A; wherein X⁶ is any amino acid other than Y; and wherein X⁷ is any amino acid other than K.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸ (SEQ ID NO:131) at the amino acids corresponding to amino acid positions 265 to 272 (VP1 numbering) of the native avian AAV capsid protein, wherein X¹ is any amino acid other than R; wherein X² is any amino acid other than I; wherein X³ is any amino acid other than Q; wherein X⁴ is any amino acid other than G; wherein X⁵ is any amino acid other than P; wherein X⁶ is any amino acid other than S; wherein X⁷ is any amino acid other than G; and wherein X⁸ is any amino acid other than G.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:132) at the amino acids corresponding to amino acid positions 375 to 384 (VP1 numbering) of the native avian AAV capsid protein, wherein X¹ is any amino acid other than I; wherein X² is any amino acid other than Y; wherein X³ is any amino acid other than T; wherein X⁴ is any amino acid other than I; wherein X⁵ is any amino acid other than P; wherein X⁶ is any amino acid other than Q; wherein X⁷ is any amino acid other than Y; wherein X⁸ is any amino acid other than G; wherein X⁹ is any amino acid other than Y; and wherein X¹⁰ is any amino acid other than C.

An adeno-associated virus (AAV) capsid protein is also provided herein, wherein the capsid protein comprises a substitution at all positions or in any combination of fewer than all positions, resulting in the amino acid sequence: X¹-X²-X³-X⁴ (SEQ ID NO:133) at the amino acids corresponding to amino acid positions 459 to 462 (VP1 numbering) of the native avian AAV capsid protein, wherein X¹ is any amino acid other than S; wherein X² is any amino acid other than S; wherein X³ is any amino acid other than G; and wherein X⁴ is any amino acid other than R.

An adeno-associated virus (AAV) capsid protein is also provided herein, wherein the capsid protein comprises a substitution at all positions or in any combination of fewer than all positions, resulting in the amino acid sequence: X¹-X²-X³-X⁵-X⁶-X⁷-X⁸-X⁹-X¹⁰-X¹¹-X¹² (SEQ ID NO:134) at the amino acids corresponding to amino acid positions 496 to 507 (VP1 numbering) of the native avian AAV capsid protein, wherein X¹ is any amino acid other than A; wherein X² is any amino acid other than S; wherein X³ is any amino acid other than N; wherein X⁴ is any amino acid other than I; wherein X⁵ is any amino acid other than T; wherein X⁶ is any amino acid other than K; wherein X⁷ is any amino acid other than N; wherein X⁸ is any amino acid other than N; wherein X⁹ is any amino acid other than V; wherein X¹⁰ is any amino acid other than F; wherein X¹¹ is any amino acid other than S; and wherein X¹² is any amino acid other than V.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷-X⁸-X⁹-X¹⁰ (SEQ ID NO:135) at the amino acids corresponding to amino acid positions 595 to 604 (VP1 numbering) of the native avian AAV capsid protein, wherein X¹ is any amino acid other than V; wherein X² is any amino acid other than T; wherein X³ is any amino acid other than P; wherein X⁴ is any amino acid other than G; wherein X⁵ is any amino acid other than T; wherein X⁶ is any amino acid other than R; wherein Vis any amino acid other than A; wherein X⁸ is any amino acid other than A; wherein X⁹ is any amino acid other than V; and wherein X¹⁰ is any amino acid other than N.

An adeno-associated virus (AAV) capsid protein is also provided herein, wherein the capsid protein comprises a substitution at all positions or in any combination of fewer than all positions, resulting in the amino acid sequence: X¹-X² at the amino acids corresponding to amino acid positions 716 to 717 (VP1 numbering) of the native avian AAV capsid protein, wherein X¹ is any amino acid other than A; and wherein X² is any amino acid other than D.

An adeno-associated virus (AAV) capsid protein is also provided herein, wherein the capsid protein comprises a substitution at all positions or in any combination of fewer than all positions, resulting in the amino acid sequence: X¹-X²-X³-X⁴-X⁵-X⁶-X⁷ (SEQ ID NO:136) at the amino acids corresponding to amino acid positions 723 to 729 (VP1 numbering) of the native avian AAV capsid protein, wherein X¹ is any amino acid other than S; wherein X² is any amino acid other than D; wherein X³ is any amino acid other than T; wherein X⁴ is any amino acid other than G; wherein X⁵ is any amino acid other than S; wherein X⁶ is any amino acid other than Y; and wherein Vis any amino acid other than S.

In embodiments wherein any amino acid residue identified as X¹ through X¹⁰ is not substituted, the amino acid residue at the unsubstituted position is the wild type amino acid residue of the reference amino acid sequence.

An AAV capsid protein is also provided herein, comprising an amino acid substitution at residues 488R, 450Q, 453S, 454G, 455S, 456A, 457Q and/or 500N of SEQ ID NO:1 (AAV1 capsid protein; VP1 numbering) in any combination.

An AAV capsid protein is also provided herein, comprising an amino acid substitution at residues 256L, 258K, 259Q, 261S, 263A, 264S, 265T, 266G, 272H, 385S, 386Q, 547S, 709A, 710N, 716D, 717N, 718N, 720L and/or 722T of SEQ ID NO:1 (AAV1 capsid protein; VP1 numbering) in any combination.

An AAV capsid protein is also provided herein, comprising an amino acid substitution at residues 244N, 246Q, 248R, 249E, 2501, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of SEQ ID NO:5 (AAV5 capsid protein; VP1 numbering).

An AAV capsid protein is also provided herein, comprising an amino acid substitution at residues 248R, 316V, 317Q, 318D, 319S, 443N, 530N, 531S, 532Q 533P, 534A, 535N, 540A, 541T, 542Y, 543L, 545G, 546N, 697Q, 704D, 706T, 708E, 709Y and/or 710R of SEQ ID NO:5 (AAV5 capsid protein; VP1 numbering) in any combination.

An AAV capsid protein is also provided herein, comprising an amino acid substitution at residues 264S, 266G, 269N, 272H, 457Q, 588S and/or 589T of SEQ ID NO:6 (AAV6 capsid protein; VP1 numbering) in any combination.

An AAV capsid protein is also provided herein, comprising an amino acid substitution at residues 457T, 459N, 496G, 499N, 500N, 589Q, 590N and/or 592A of SEQ ID NO:8 (AAV8 capsid protein; VP1 numbering) in any combination.

An AAV capsid protein is also provided herein, comprising an amino acid substitution at residues 4511, 452N, 453G, 454S, 455G, 456Q, 457N and/or 458Q of SEQ ID NO:9 (AAV9 capsid protein; VP1 numbering) in any combination.

An AAV capsid protein is also provided herein, comprising a S472R substitution in the amino acid sequence of SEQ ID NO:1 (AAV1 capsid protein; VP1 numbering).

An AAV capsid protein is also provided herein, comprising a V473D substitution in the amino acid sequence of SEQ ID NO:1 (AAV1 capsid protein; VP1 numbering).

An AAV capsid protein is also provided herein, comprising a N500E substitution in the amino acid sequence of SEQ ID NO:1 (AAV1 capsid protein; VP1 numbering).

An AAV capsid protein is also provided herein, comprising an A456T, Q457T, N458Q and K459S substitution in the amino acid sequence of SEQ ID NO:1 (AAV1 capsid protein; VP1 numbering).

An AAV capsid protein is also provided herein, comprising a T492S and K493A substitution in the amino acid sequence of SEQ ID NO:1 (AAV1 capsid protein; VP1 numbering).

An AAV capsid protein is also provided herein, comprising a S586R, S587G, S588N and T589R substitution in the amino acid sequence of SEQ ID NO:1 (AAV1 capsid protein; VP1 numbering).

An AAV capsid protein is also provided herein, comprising an A456T, Q457T, N458Q, K4595, T492S and K493A substitution in the amino acid sequence of SEQ ID NO:1 (AAV1 capsid protein; VP1 numbering).

An AAV capsid protein is also provided herein, comprising an A456T, Q457T, N458Q, K4595, S586R, S587G, S588N and T589R substitution in the amino acid sequence of SEQ ID NO:1 (AAV1 capsid protein; VP1 numbering).

An AAV capsid protein is also provided herein, comprising a T492S, K493A, S586R, S587G, S588N and T589R substitution in the amino acid sequence of SEQ ID NO:1 (AAV1 capsid protein; VP1 numbering).

An AAV capsid protein is also provided herein, comprising an A456T, Q457T, N458Q, K459S, T492S, K493A, S586R, S587G, S588N and T589R substitution in the amino acid sequence of SEQ ID NO:1 (AAV1 capsid protein; VP1 numbering).

The present invention further provides an AAV capsid protein comprising one or more amino acid substitutions of this invention, in any combination. For example, an AAV capsid protein of any given serotype described herein can comprise substitutions at the amino acid residues identified for CAM1, CAM3, CAM4-1, CAM4-2, CAMS, CAM6, CAM7, CAMS, CAMS-1 and/or CAMS-2 (listed in Table 5), singly or in any combination. As a further example, an AAV capsid of a first serotype can comprise amino acid substitutions that introduce residues that define a CAM region of a different AAV serotype, which can be a second, third, fourth AAV serotype, etc. The CAM regions of different AAV serotypes can be present on a first AAV serotype in any combination. This cumulative approach generates novel AAVe strains, which present variable antigenic surface topologies that would evade neutralizing antibodies. As a particular, nonlimiting example, an AAV1 serotype capsid protein can comprise an endogenous or mutated CAM1 region from a different second AAV serotype and an endogenous or mutated CAM3 region of a different third serotype and an endogenous or mutated CAM4 region of a different fourth serotype, etc., in any combination, as would be recognized by one of ordinary skill in the art.

In particular embodiments, the modified virus capsid proteins of the invention are not limited to AAV capsid proteins in which amino acids from one AAV capsid protein are substituted into another AAV capsid protein, and the substituted and/or inserted amino acids can be from any source, and can further be naturally occurring or partially or completely synthetic.

As described herein, the nucleic acid and amino acid sequences of the capsid proteins from a number of AAV are known in the art. Thus, the amino acids “corresponding” to amino acid positions of the native AAV capsid protein can be readily determined for any other AAV (e.g., by using sequence alignments).

The invention contemplates that the modified capsid proteins of the invention can be produced by modifying the capsid protein of any AAV now known or later discovered. Further, the AAV capsid protein that is to be modified can be a naturally occurring AAV capsid protein (e.g., an AAV2, AAV3a or 3b, AAV4, AAV5, AAV8, AAV9, AAV10 or AAV11 capsid protein or any of the AAV shown in Table 1) but is not so limited. Those skilled in the art will understand that a variety of manipulations to the AAV capsid proteins are known in the art and the invention is not limited to modifications of naturally occurring AAV capsid proteins. For example, the capsid protein to be modified may already have alterations as compared with naturally occurring AAV (e.g., is derived from a naturally occurring AAV capsid protein, e.g., AAV2, AAV3a, AAV3b, AAV4, AAV5, AAV6, AAV7, AAV8, AAV9, AAV10, AAV11, AAV12 or any other AAV now known or later discovered). Such AAV capsid proteins are also within the scope of the present invention.

Thus, in particular embodiments, the AAV capsid protein to be modified can be derived from a naturally occurring AAV but further comprise one or more foreign sequences (e.g., that are exogenous to the native virus) that are inserted and/or substituted into the capsid protein and/or has been altered by deletion of one or more amino acids.

Accordingly, when referring herein to a specific AAV capsid protein (e.g., an AAV2, AAV3, AAV4, AAV5, AAV6, AAV7, AAV8, AAV9, AAV10 or AAV11 capsid protein or a capsid protein from any of the AAV shown in Table 1, etc.), it is intended to encompass the native capsid protein as well as capsid proteins that have alterations other than the modifications of the invention. Such alterations include substitutions, insertions and/or deletions. In particular embodiments, the capsid protein comprises 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acids inserted therein (other than the insertions of the present invention) as compared with the native AAV capsid protein sequence. In embodiments of the invention, the capsid protein comprises 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acid substitutions (other than the amino acid substitutions according to the present invention) as compared with the native AAV capsid protein sequence. In embodiments of the invention, the capsid protein comprises a deletion of 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acids (other than the amino acid deletions of the invention) as compared with the native AAV capsid protein sequence.

Thus, for example, the term “AAV2 capsid protein” includes AAV capsid proteins having the native AAV2 capsid protein sequence (see GenBank Accession No. AAC03780) as well as those comprising substitutions, insertions and/or deletions (as described in the preceding paragraph) in the native AAV2 capsid protein sequence.

In particular embodiments, the AAV capsid protein has the native AAV capsid protein sequence or has an amino acid sequence that is at least about 90%, 95%, 97%, 98% or 99% similar or identical to a native AAV capsid protein sequence. For example, in particular embodiments, an “AAV2” capsid protein encompasses the native AAV2 capsid protein sequence as well as sequences that are at least about 90%, 95%, 97%, 98% or 99% similar or identical to the native AAV2 capsid protein sequence.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

The invention also provides a virus capsid comprising, consisting essentially of, or consisting of the modified AAV capsid protein of the invention. In particular embodiments, the virus capsid is a parvovirus capsid, which may further be an autonomous parvovirus capsid or a dependovirus capsid. Optionally, the virus capsid is an AAV capsid. In particular embodiments, the AAV capsid is an AAV1, AAV2, AAV3a, AAV3b, AAV4, AAV5, AAV6, AAV7, AAV8, AAV9, AAV10, AAV11, AAV12, AAVrh8, AAVrh10, AAVrh32.33, bovine AAV capsid, avian AAV capsid or any other AAV now known or later identified. A nonlimiting list of AAV serotypes is shown in Table 1 an AAV capsid of this invention can be any AAV serotype listed in Table 1 or derived from any of the foregoing by one or more insertions, substitutions and/or deletions.

The modified virus capsids can be used as “capsid vehicles,” as has been described, for example, in U.S. Pat. No. 5,863,541. Molecules that can be packaged by the modified virus capsid and transferred into a cell include heterologous DNA, RNA, polypeptides, small organic molecules, metals, or combinations of the same.

Heterologous molecules are defined as those that are not naturally found in an AAV infection, e.g., those not encoded by a wild-type AAV genome. Further, therapeutically useful molecules can be associated with the outside of the chimeric virus capsid for transfer of the molecules into host target cells. Such associated molecules can include DNA, RNA, small organic molecules, metals, carbohydrates, lipids and/or polypeptides. In one embodiment of the invention the therapeutically useful molecule is covalently linked (i.e., conjugated or chemically coupled) to the capsid proteins. Methods of covalently linking molecules are known by those skilled in the art.

The modified virus capsids of the invention also find use in raising antibodies against the novel capsid structures. As a further alternative, an exogenous amino acid sequence may be inserted into the modified virus capsid for antigen presentation to a cell, e.g., for administration to a subject to produce an immune response to the exogenous amino acid sequence.

In other embodiments, the virus capsids can be administered to block certain cellular sites prior to and/or concurrently with (e.g., within minutes or hours of each other) administration of a virus vector delivering a nucleic acid encoding a polypeptide or functional RNA of interest. For example, the inventive capsids can be delivered to block cellular receptors on liver cells and a delivery vector can be administered subsequently or concurrently, which may reduce transduction of liver cells, and enhance transduction of other targets (e.g., skeletal, cardiac and/or diaphragm muscle).

According to representative embodiments, modified virus capsids can be administered to a subject prior to and/or concurrently with a modified virus vector according to the present invention. Further, the invention provides compositions and pharmaceutical formulations comprising the inventive modified virus capsids; optionally, the composition also comprises a modified virus vector of the invention.

The invention also provides nucleic acids (optionally, isolated nucleic acids) encoding the modified virus capsids and capsid proteins of the invention. Further provided are vectors comprising the nucleic acids, and cells (in vivo or in culture) comprising the nucleic acids and/or vectors of the invention. As one example, the present invention provides a virus vector comprising: (a) a modified AAV capsid of this invention; and (b) a nucleic acid comprising at least one terminal repeat sequence, wherein the nucleic acid is encapsidated by the AAV capsid.

Other suitable vectors include without limitation viral vectors (e.g., adenovirus, AAV, herpesvirus, vaccinia, poxviruses, baculoviruses, and the like), plasmids, phage, YACs, BACs, and the like. Such nucleic acids, vectors and cells can be used, for example, as reagents (e.g., helper packaging constructs or packaging cells) for the production of modified virus capsids or virus vectors as described herein.

Virus capsids according to the invention can be produced using any method known in the art, e.g., by expression from a baculovirus (Brown et al., (1994) Virology 198:477-488). The modifications to the AAV capsid protein according to the present invention are “selective” modifications. This approach is in contrast to previous work with whole subunit or large domain swaps between AAV serotypes (see, e.g., international patent publication WO 00/28004 and Hauck et al., (2003) J. Virology 77:2768-2774). In particular embodiments, a “selective” modification results in the insertion and/or substitution and/or deletion of less than about 20, 18, 15, 12, 10, 9, 8, 7, 6, 5, 4 or 3 contiguous amino acids.

The modified capsid proteins and capsids of the invention can further comprise any other modification, now known or later identified.

For example, the AAV capsid proteins and virus capsids of the invention can be chimeric in that they can comprise all or a portion of a capsid subunit from another virus, optionally another parvovirus or AAV, e.g., as described in international patent publication WO 00/28004.

In some embodiments of this invention, the virus capsid can be a targeted virus capsid, comprising a targeting sequence (e.g., substituted or inserted in the viral capsid) that directs the virus capsid to interact with cell-surface molecules present on desired target tissue(s) (see, e.g., international patent publication WO 00/28004 and Hauck et al., (2003) J. Virology 77:2768-2774); Shi et al., Human Gene Therapy 17:353-361 (2006) [describing insertion of the integrin receptor binding motif RGD at positions 520 and/or 584 of the AAV capsid subunit]; and U.S. Pat. No. 7,314,912 [describing insertion of the P1 peptide containing an RGD motif following amino acid positions 447, 534, 573 and 587 of the AAV2 capsid subunit]). Other positions within the AAV capsid subunit that tolerate insertions are known in the art (e.g., positions 449 and 588 described by Grifman et al., Molecular Therapy 3:964-975 (2001)).

For example, a virus capsid of this invention may have relatively inefficient tropism toward certain target tissues of interest (e.g., liver, skeletal muscle, heart, diaphragm muscle, kidney, brain, stomach, intestines, skin, endothelial cells, and/or lungs). A targeting sequence can advantageously be incorporated into these low-transduction vectors to thereby confer to the virus capsid a desired tropism and, optionally, selective tropism for particular tissue(s). AAV capsid proteins, capsids and vectors comprising targeting sequences are described, for example in international patent publication WO 00/28004. As another example, one or more non-naturally occurring amino acids as described by Wang et al., Annu Rev Biophys Biomol Struct. 35:225-49 (2006)) can be incorporated into an AAV capsid subunit of this invention at an orthogonal site as a means of redirecting a low-transduction vector to desired target tissue(s). These unnatural amino acids can advantageously be used to chemically link molecules of interest to the AAV capsid protein including without limitation: glycans (mannose—dendritic cell targeting); RGD, bombesin or a neuropeptide for targeted delivery to specific cancer cell types; RNA aptamers or peptides selected from phage display targeted to specific cell surface receptors such as growth factor receptors, integrins, and the like. Methods of chemically modifying amino acids are known in the art (see, e.g., Greg T. Hermanson, Bioconjugate Techniques, 1^{st} edition, Academic Press, 1996).

In some embodiments, the targeting sequence may be a virus capsid sequence (e.g., an autonomous parvovirus capsid sequence, AAV capsid sequence, or any other viral capsid sequence) that directs infection to a particular cell type(s).

As another nonlimiting example, a heparin binding domain (e.g., the respiratory syncytial virus heparin binding domain) may be inserted or substituted into a capsid subunit that does not typically bind HS receptors (e.g., AAV 4, AAV5) to confer heparin binding to the resulting mutant.

B19 infects primary erythroid progenitor cells using globoside as its receptor (Brown et al., (1993) Science 262:114). The structure of B19 has been determined to 8 Å resolution (Agbandje-McKenna et al., (1994) Virology 203:106). The region of the B19 capsid that binds to globoside has been mapped between amino acids 399-406 (Chapman et al., (1993) Virology 194:419), a looped out region between n-barrel structures E and F (Chipman et al., (1996) Proc. Nat. Acad. Sci. USA 93:7502). Accordingly, the globoside receptor binding domain of the B19 capsid may be substituted into an AAV capsid protein of this invention to target a virus capsid or virus vector comprising the same to erythroid cells.

In some embodiments, the exogenous targeting sequence may be any amino acid sequence encoding a peptide that alters the tropism of a virus capsid or virus vector comprising the modified AAV capsid protein. In particular embodiments, the targeting peptide or protein may be naturally occurring or, alternately, completely or partially synthetic. Exemplary targeting sequences include ligands and other peptides that bind to cell surface receptors and glycoproteins, such as RGD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., α, β or γ), neuropeptides and endorphins, and the like, and fragments thereof that retain the ability to target cells to their cognate receptors. Other illustrative peptides and proteins include substance P, keratinocyte growth factor, neuropeptide Y, gastrin releasing peptide, interleukin 2, hen egg white lysozyme, erythropoietin, gonadoliberin, corticostatin, β-endorphin, leu-enkephalin, rimorphin, a-neo-enkephalin, angiotensin, pneumadin, vasoactive intestinal peptide, neurotensin, motilin, and fragments thereof as described above. As yet a further alternative, the binding domain from a toxin (e.g., tetanus toxin or snake toxins, such as a-bungarotoxin, and the like) can be substituted into the capsid protein as a targeting sequence. In a yet further representative embodiment, the AAV capsid protein can be modified by substitution of a “nonclassical” import/export signal peptide (e.g., fibroblast growth factor-1 and -2, interleukin 1, HIV-1 Tat protein, herpes virus VP22 protein, and the like) as described by Cleves (Current Biology 7:R318 (1997)) into the AAV capsid protein. Also encompassed are peptide motifs that direct uptake by specific cells, e.g., a FVFLP (SEQ ID NO:162) peptide motif triggers uptake by liver cells.

Phage display techniques, as well as other techniques known in the art, may be used to identify peptides that recognize any cell type of interest.

The targeting sequence may encode any peptide that targets to a cell surface binding site, including receptors (e.g., protein, carbohydrate, glycoprotein or proteoglycan). Examples of cell surface binding sites include, but are not limited to, heparan sulfate, chondroitin sulfate, and other glycosaminoglycans, sialic acid moieties found on mucins, glycoproteins, and gangliosides, MHC I glycoproteins, carbohydrate components found on membrane glycoproteins, including, mannose, N-acetyl-galactosamine, N-acetyl-glucosamine, fucose, galactose, and the like.

In particular embodiments, a heparan sulfate (HS) or heparin binding domain is substituted into the virus capsid (for example, in an AAV capsid that otherwise does not bind to HS or heparin). It is known in the art that HS/heparin binding is mediated by a “basic patch” that is rich in arginines and/or lysines. In exemplary embodiments, a sequence following the motif BXXB (SEQ ID NO:163), where “B” is a basic residue and X is neutral and/or hydrophobic can be employed. As a nonlimiting example, BXXB can be RGNR (SEQ ID NO:164). As another nonlimiting example, BXXB is substituted for amino acid positions 262 through 265 in the native AAV2 capsid protein or at the corresponding position(s) in the capsid protein of another AAV serotype.

Other nonlimiting examples of suitable targeting sequences include the peptides targeting coronary artery endothelial cells identified by Willer et al., Nature Biotechnology 21:1040-1046 (2003) (consensus sequences NSVRDL(G/S) (SEQ ID NO:165), PRSVTVP (SEQ ID NO:166), NSVSSX(S/A) (SEQ ID NO:167); tumor-targeting peptides as described by Grifman et al., Molecular Therapy 3:964-975 (2001) (e.g., NGR, NGRAHA, SEQ ID NO:168); lung or brain targeting sequences as described by Work et al., Molecular Therapy 13:683-693 (2006) (QPEHSST; SEQ ID NO:169, VNTANST; SEQ ID NO:170, HGPMQKS; SEQ ID NO:171, PHKPPLA; SEQ ID NO:172, IKNNEMW; SEQ ID NO:173, RNLDTPM; SEQ ID NO:174, VDSHRQS; SEQ ID NO:175, YDSKTKT; SEQ ID NO:176, SQLPHQK; SEQ ID NO:177, STMQQNT; SEQ ID NO:178, TERYMTQ; SEQ ID NO:179, QPEHSST; SEQ ID NO:180, DASLSTS; SEQ ID NO:181, DLPNKKT; SEQ ID NO:182, DLTAARL; SEQ ID NO:183, EPHQFNY; SEQ ID NO:184, EPQSNHT; SEQ ID NO:185, MSSWPSQ; SEQ ID NO:186, NPKHNAT; SEQ ID NO:187, PDGMRTT; SEQ ID NO:188, PNNNKTT; SEQ ID NO:189, QSTTHDS; SEQ ID NO:190, TGSKQKQ; SEQ ID NO:191, SLKHQAL; SEQ ID NO:192 and SPIDGEQ; SEQ ID NO:193); vascular targeting sequences described by Hajitou et al., TCM 16:80-88 (2006) (WIFPWIQL; SEQ ID NO:194, CDCRGDCFC; SEQ ID NO:195, CNGRC; SEQ ID NO:196, CPRECES; SEQ ID NO:197, GSL, CTTHWGFTLC; SEQ ID NO:198, CGRRAGGSC; SEQ ID NO:199, CKGGRAKDC; SEQ ID NO:200, and CVPELGHEC; SEQ ID NO:201); targeting peptides as described by Koivunen et al., J. Nucl. Med. 40:883-888 (1999) (CRRETAWAK; SEQ ID NO:202, KGD, VSWFSHRYSPFAVS; SEQ ID NO:203, GYRDGYAGPILYN; SEQ ID NO:204, XXXY*XXX (SEQ ID NO:205) [where Y* is phospho-Tyr], Y*E/MNW; SEQ ID NO:206, RPLPPLP; SEQ ID NO:207, APPLPPR; SEQ ID NO:208, DVFYPYPYASGS; SEQ ID NO:209, MYWYPY; SEQ ID NO:210, DITWDQLWDLMK; SEQ ID NO:211, CWDD(G/L)WLC; SEQ ID NO:212, EWCEYLGGYLRCYA; SEQ ID NO:213, YXCXXGPXTWXCXP; SEQ ID NO:214, IEGPTLRQWLAARA; SEQ ID NO:215, LWXX(Y/W/F/H); SEQ ID NO:216, XFXXYLW; SEQ ID NO:217, SSIISHFRWGLCD; SEQ ID NO:218, MSRPACPPNDKYE; SEQ ID NO:219, CLRSGRGC; SEQ ID NO:220, CHWMFSPWC; SEQ ID NO:221, WXXF; SEQ ID NO:222, CSSRLDAC; SEQ ID NO:223, CLPVASC; SEQ ID NO:224, CGFECVRQCPERC; SEQ ID NO:225, CVALCREACGEGC; SEQ ID NO:226, SWCEPGWCR; SEQ ID NO:227, YSGKWGW; SEQ ID NO:228, GLSGGRS; SEQ ID NO:229, LMLPRAD; SEQ ID NO:230, CSCFRDVCC; SEQ ID NO:231, CRDVVSVIC; SEQ ID NO:232, CNGRC; SEQ ID NO:233, and GSL); and tumor targeting peptides as described by Newton & Deutscher, Phage Peptide Display in Handbook of Experimental Pharmacology, pages 145-163, Springer-Verlag, Berlin (2008) (MARSGL; SEQ ID NO:234, MARAKE; SEQ ID NO:235, MSRTMS; SEQ ID NO:236, KCCYSL; SEQ ID NO:237, WRR, WKR, WVR, WVK, WIK, WTR, WVL, WLL, WRT, WRG, WVS, WVA, MYWGDSHWLQYWYE; SEQ ID NO:238, MQLPLAT; SEQ ID NO:239, EWLS; SEQ ID NO:240, SNEW; SEQ ID NO:241, TNYL; SEQ ID NO:242, WIFPWIQL; SEQ ID NO:243, WDLAWMFRLPVG; SEQ ID NO:244, CTVALPGGYVRVC; SEQ ID NO:245, CVPELGHEC; SEQ ID NO:246, CGRRAGGSC; SEQ ID NO:247, CVAYCIEHHCWTC; SEQ ID NO:248, CVFAHNYDYLVC; SEQ ID NO:249, and CVFTSNYAFC; SEQ ID NO:250, VHSPNKK; SEQ ID NO:251, CDCRGDCFC; SEQ ID NO:252, CRGDGWC; SEQ ID NO:253, XRGCDX; SEQ ID NO:254, PXX(S/T); SEQ ID NO:255, CTTHWGFTLC; SEQ ID NO:256, SGKGPRQITAL; SEQ ID NO:257, A(A/Q)(N/A)(L/Y)(T/V/M/R)(R/K); SEQ ID NO:258, VYMSPF; SEQ ID NO:259, MQLPLAT; SEQ ID NO:260, ATWLPPR; SEQ ID NO:261, HTMYYHHYQHHL; SEQ ID NO:262, SEVGCRAGPLQWLCEKYFG; SEQ ID NO:263, CGLLPVGRPDRNVWRWLC; SEQ ID NO:264, CKGQCDRFKGLPWEC; SEQ ID NO:265, SGRSA; SEQ ID NO:266, WGFP; SEQ ID NO:267, LWXXAr [Ar=Y, W, F, H); SEQ ID NO:216, XFXXYLW; SEQ ID NO:268, AEPMPHSLNFSQYLWYT; SEQ ID NO:269, WAY(W/F)SP; SEQ ID NO:270, IELLQAR; SEQ ID NO:271, DITWDQLWDLMK; SEQ ID NO:272, AYTKCSRQWRTCMTTH; SEQ ID NO:273, PQNSKIPGPTFLDPH; SEQ ID NO:274, SMEPALPDWWWKMFK; SEQ ID NO:275, ANTPCGPYTHDCPVKR; SEQ ID NO:276, TACHQHVRMVRP; SEQ ID NO:277, VPWMEPAYQRFL; SEQ ID NO:278, DPRATPGS; SEQ ID NO:279, FRPNRAQDYNTN; SEQ ID NO:280, CTKNSYLMC; SEQ ID NO:281, C(R/Q)L/RT(G/N)XXG(A/V)GC; SEQ ID NO:282, CPIEDRPMC; SEQ ID NO:283, HEWSYLAPYPWF; SEQ ID NO:284, MCPKHPLGC; SEQ ID NO:285, RMWPSSTVNLSAGRR; SEQ ID NO:286, SAKTAVSQRVWLPSHRGGEP; SEQ ID NO:287, KSREHVNNSACPSKRITAAL; SEQ ID NO:288, EGFR; SEQ ID NO:289, RVS, AGS, AGLGVR; SEQ ID NO:290, GGR, GGL, GSV, GVS, GTRQGHTMRLGVSDG; SEQ ID NO:291, IAGLATPGWSHWLAL; SEQ ID NO:292, SMSIARL; SEQ ID NO:293, HTFEPGV; SEQ ID NO:294, NTSLKRISNKRIRRK; SEQ ID NO:295, LRIKRKRRKRKKTRK; SEQ ID NO:296, GGG, GFS, LWS, EGG, LLV, LSP, LBS, AGG, GRR, GGH and GTV).

As yet a further embodiment, the targeting sequence may be a peptide that can be used for chemical coupling (e.g., can comprise arginine and/or lysine residues that can be chemically coupled through their R groups) to another molecule that targets entry into a cell.

As another embodiment, the AAV capsid protein or virus capsid of the invention can comprise a mutation as described in WO 2006/066066. For example, the capsid protein can comprise a selective amino acid substitution at amino acid position 263, 705, 708 and/or 716 of the native AAV2 capsid protein or a corresponding change(s) in a capsid protein from another AAV serotype.

Additionally, or alternatively, in representative embodiments, the capsid protein, virus capsid or vector comprises a selective amino acid insertion directly following amino acid position 264 of the AAV2 capsid protein or a corresponding change in the capsid protein from other AAV. By “directly following amino acid position X” it is intended that the insertion immediately follows the indicated amino acid position (for example, “following amino acid position 264” indicates a point insertion at position 265 or a larger insertion, e.g., from positions 265 to 268, etc.).

Furthermore, in representative embodiments, the capsid protein, virus capsid or vector of this invention can comprise amino acid modifications such as described in PCT Publication No. WO 2010/093784 (e.g., 2i8) and/or in PCT Publication No. WO 2014/144229 (e.g., dual glycan).

In some embodiments of this invention, the capsid protein, virus capsid or vector of this invention can have equivalent or enhanced transduction efficiency relative to the transduction efficiency of the AAV serotype from which the capsid protein, virus capsid or vector of this invention originated. In some embodiments of this invention, the capsid protein, virus capsid or vector of this invention can have reduced transduction efficiency relative to the transduction efficiency of the AAV serotype from which the capsid protein, virus capsid or vector of this invention originated. In some embodiments of this invention, the capsid protein, virus capsid or vector of this invention can have equivalent or enhanced tropism relative to the tropism of the AAV serotype from which the capsid protein, virus capsid or vector of this invention originated. In some embodiments of this invention, the capsid protein, virus capsid or vector of this invention can have an altered or different tropism relative to the tropism of the AAV serotype from which the capsid protein, virus capsid or vector of this invention originated.

In some embodiments of this invention, the capsid protein, virus capsid or vector of this invention can have or be engineered to have tropism for brain tissue.

The foregoing embodiments of the invention can be used to deliver a heterologous nucleic acid to a cell or subject as described herein. For example, the modified vector can be used to treat a lysosomal storage disorder such as a mucopolysaccharidosis disorder (e.g., Sly syndrome [β-glucuronidase], Hurler Syndrome [α-L-iduronidase], Scheie Syndrome [α-L-iduronidase], Hurler-Scheie Syndrome [α-L-iduronidase], Hunter's Syndrome [iduronate sulfatase], Sanfilippo Syndrome A [heparan sulfamidase], B [N-acetylglucosaminidase], C [acetyl-CoA:α-glucosaminide acetyltransferase], D [N-acetylglucosamine 6-sulfatase], Morquio Syndrome A [galactose-6-sulfate sulfatase], B [β-galactosidase], Maroteaux-Lamy Syndrome [N-acetylgalactosamine-4-sulfatase], etc.), Fabry disease (a-galactosidase), Gaucher's disease (glucocerebrosidase), or a glycogen storage disorder (e.g., Pompe disease; lysosomal acid α-glucosidase) as described herein.

Those skilled in the art will appreciate that for some AAV capsid proteins the corresponding modification will be an insertion and/or a substitution, depending on whether the corresponding amino acid positions are partially or completely present in the virus or, alternatively, are completely absent. Likewise, when modifying AAV other than AAV2, the specific amino acid position(s) may be different than the position in AAV2 (see, e.g., Table 4). As discussed elsewhere herein, the corresponding amino acid position(s) will be readily apparent to those skilled in the art using well-known techniques.

Nonlimiting examples of corresponding positions in a number of other AAV are shown in Table 4 (Position 2). In particular embodiments, the amino acid insertion or substitution is a threonine, aspartic acid, glutamic acid or phenylalanine (excepting AAV that have a threonine, glutamic acid or phenylalanine, respectively, at this position).

In other representative embodiments, the modified capsid proteins or virus capsids of the invention further comprise one or more mutations as described in WO 2007/089632 (e.g., an E→K mutation at amino acid position 531 of the AAV2 capsid protein or the corresponding position of the capsid protein from another AAV).

In further embodiments, the modified capsid protein or capsid can comprise a mutation as described in WO 2009/108274.

As another, possibility, the AAV capsid protein can comprise a mutation as described by Zhong et al. (Virology 381: 194-202 (2008); Proc. Nat. Acad. Sci. 105: 7827-32 (2008)). For example, the AAV capsid protein can comprise a Y→F mutation at amino acid position 730.

The modifications described above can be incorporated into the capsid proteins or capsids of the invention in combination with each other and/or with any other modification now known or later discovered.

TABLE 4

| Serotype | Position 1 | Position 2 |
|---|---|---|
| AAV1 | A263X | T265X |
| AAV2 | Q263X | —265X |
| AAV3A | Q263X | —265X |
| AAV3B | Q263X | —265X |
| AAV4 | S257X | —259X |
| AAV5 | G253X | V255X |
| AAV6 | A263X | T265X |
| AAV7 | E264X | A266X |
| AAV8 | G264X | S266X |
| AAV9 | S263X | S265X |

Where, (X) → mutation to any amino acid; (—) → insertion of any amino acid
Note:
Position 2 inserts are indicated by the site of insertion

The invention also encompasses virus vectors comprising the modified capsid proteins and capsids of the invention. In particular embodiments, the virus vector is a parvovirus vector (e.g., comprising a parvovirus capsid and/or vector genome), for example, an AAV vector (e.g., comprising an AAV capsid and/or vector genome). In representative embodiments, the virus vector comprises a modified AAV capsid comprising a modified capsid subunit of the invention and a vector genome.

For example, in representative embodiments, the virus vector comprises: (a) a modified virus capsid (e.g., a modified AAV capsid) comprising a modified capsid protein of the invention; and (b) a nucleic acid comprising a terminal repeat sequence (e.g., an AAV TR), wherein the nucleic acid comprising the terminal repeat sequence is encapsidated by the modified virus capsid. The nucleic acid can optionally comprise two terminal repeats (e.g., two AAV TRs).

In representative embodiments, the virus vector is a recombinant virus vector comprising a heterologous nucleic acid encoding a polypeptide or functional RNA of interest. Recombinant virus vectors are described in more detail below.

In particular embodiments, the virus vectors of the invention (i) have reduced transduction of liver as compared with the level of transduction by a virus vector without the modified capsid protein; (ii) exhibit enhanced systemic transduction by the virus vector in an animal subject as compared with the level observed by a virus vector without the modified capsid protein; (iii) demonstrate enhanced movement across endothelial cells as compared with the level of movement by a virus vector without the modified capsid protein, and/or (iv) exhibit a selective enhancement in transduction of muscle tissue (e.g., skeletal muscle, cardiac muscle and/or diaphragm muscle), and/or (v) reduced transduction of brain tissues (e.g., neurons) as compared with the level of transduction by a virus vector without the modified capsid protein. In particular embodiments, the virus vector has systemic transduction toward muscle, e.g., transduces multiple skeletal muscle groups throughout the body and optionally transduces cardiac muscle and/or diaphragm muscle.

It will be understood by those skilled in the art that the modified capsid proteins, virus capsids and virus vectors of the invention exclude those capsid proteins, capsids and virus vectors that have the indicated amino acids at the specified positions in their native state (i.e., are not mutants).

### Methods of Producing Virus Vectors.

The present invention further provides methods of producing the inventive virus vectors. Thus, in one embodiment, the present invention provides a method of producing an AAV vector that evades neutralizing antibodies, comprising: a) identifying contact amino acid residues that form a three dimensional antigenic footprint on an AAV capsid protein; b) generating a library of AAV capsid proteins comprising amino acid substitutions of the contact amino acid residues identified in (a); c) producing AAV particles comprising capsid proteins from the library of AAV capsid proteins of (b); d) contacting the AAV particles of (c) with cells under conditions whereby infection and replication can occur; e) selecting AAV particles that can complete at least one infectious cycle and replicate to titers similar to control AAV particles; f) contacting the AAV particles selected in (e) with neutralizing antibodies and cells under conditions whereby infection and replication can occur; and g) selecting AAV particles that are not neutralized by the neutralizing antibodies of (f) Nonlimiting examples of methods for identifying contact amino acid residues include peptide epitope mapping and/or cryo-electron microscopy.

Resolution and identification of the antibody contact residues within the three dimensional antigenic footprint allows for their subsequent modification through random, rational and/or degenerate mutagenesis to generate antibody-evading AAV capsids that can be identified through further selection and/or screening.

Thus, in a further embodiment, the present invention provides a method of producing an AAV vector that evades neutralizing antibodies, comprising: a) identifying contact amino acid residues that form a three dimensional antigenic footprint on an AAV capsid protein; b) generating AAV capsid proteins comprising amino acid substitutions of the contact amino acid residues identified in (a) by random, rational and/or degenerate mutagenesis; c) producing AAV particles comprising capsid proteins from the AAV capsid proteins of (b); d) contacting the AAV particles of (c) with cells under conditions whereby infection and replication can occur; e) selecting AAV particles that can complete at least one infectious cycle and replicate to titers similar to control AAV particles; f) contacting the AAV particles selected in (e) with neutralizing antibodies and cells under conditions whereby infection and replication can occur; and g) selecting AAV particles that are not neutralized by the neutralizing antibodies of (f)

Nonlimiting examples of methods for identifying contact amino acid residues include peptide epitope mapping and/or cryo-electron microscopy. Methods of generating AAV capsid proteins comprising amino acid substitutions of contact amino acid residues by random, rational and/or degenerate mutagenesis are known in the art.

This comprehensive approach presents a platform technology that can be applied to modifying any AAV capsid. Application of this platform technology yields AAV antigenic variants derived from the original AAV capsid template without loss of transduction efficiency. As one advantage and benefit, application of this technology will expand the cohort of patients eligible for gene therapy with AAV vectors.

In one embodiment, the present invention provides a method of producing a virus vector, the method comprising providing to a cell: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids of the invention). Optionally, the nucleic acid template further comprises at least one heterologous nucleic acid sequence. In particular embodiments, the nucleic acid template comprises two AAV ITR sequences, which are located 5′ and 3′ to the heterologous nucleic acid sequence (if present), although they need not be directly contiguous thereto.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells.

The cell can be a cell that is permissive for AAV viral replication. Any suitable cell known in the art may be employed. In particular embodiments, the cell is a mammalian cell. As another option, the cell can be a trans-complementing packaging cell line that provides functions deleted from a replication-defective helper virus, e.g., 293 cells or other Ela trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences may be stably incorporated into a cell.

Typically the AAV rep/cap sequences will not be flanked by the TRs, to prevent rescue and/or packaging of these sequences.

The nucleic acid template can be provided to the cell using any method known in the art. For example, the template can be supplied by a non-viral (e.g., plasmid) or viral vector. In particular embodiments, the nucleic acid template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the nucleic acid template is provided by a replicating rAAV virus. In still other embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector further can further comprise the nucleic acid template. The AAV rep/cap sequences and/or the rAAV template can be inserted into a deleted region (e.g., the Ela or E3 regions) of the adenovirus.

In a further embodiment, the AAV rep/cap sequences and the adenovirus helper sequences are supplied by a single adenovirus helper vector. According to this embodiment, the rAAV template can be provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions.

Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Therapy 6:986 and WO 00/17377.

As a further alternative, the virus vectors of the invention can be produced in insect cells using baculovirus vectors to deliver the rep/cap genes and rAAV template as described, for example, by Urabe et al., (2002) Human Gene Therapy 13:1935-43.

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

### Recombinant Virus Vectors.

The virus vectors of the present invention are useful for the delivery of nucleic acids to cells in vitro, ex vivo, and in vivo. In particular, the virus vectors can be advantageously employed to deliver or transfer nucleic acids to animal, including mammalian, cells.

Any heterologous nucleic acid sequence(s) of interest may be delivered in the virus vectors of the present invention. Nucleic acids of interest include nucleic acids encoding polypeptides, including therapeutic (e.g., for medical or veterinary uses) or immunogenic (e.g., for vaccines) polypeptides.

Therapeutic polypeptides include, but are not limited to, cystic fibrosis transmembrane regulator protein (CFTR), dystrophin (including mini- and micro-dystrophins, see, e.g., Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, a-galactosidase A, receptors (e.g., the tumor necrosis growth factorα soluble receptor), S100-X¹, parvalbumin, adenylyl cyclase type 6, a molecule that modulates calcium handling (e.g., SERCA_{2A}, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172), angiogenesis inhibitors such as Vasohibins and other VEGF inhibitors (e.g., Vasohibin 2 [see, WO JP2006/073052]). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al., Nature Biotechnology 23:584-590 (2005)).

Heterologous nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Optionally, the heterologous nucleic acid encodes a secreted polypeptide (e.g., a polypeptide that is a secreted polypeptide in its native state or that has been engineered to be secreted, for example, by operable association with a secretory signal sequence as is known in the art).

Alternatively, in particular embodiments of this invention, the heterologous nucleic acid may encode an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

Further, a nucleic acid sequence that directs alternative splicing can be delivered. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a modified capsid of the invention.

The virus vector may also comprise a heterologous nucleic acid that shares homology with and recombines with a locus on a host chromosome. This approach can be utilized, for example, to correct a genetic defect in the host cell.

The present invention also provides virus vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.). The antigen may be presented in the parvovirus capsid. Alternatively, the antigen may be expressed from a heterologous nucleic acid introduced into a recombinant vector genome. Any immunogen of interest as described herein and/or as is known in the art can be provided by the virus vector of the present invention.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, P-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

As a further alternative, the heterologous nucleic acid can encode any polypeptide that is desirably produced in a cell in vitro, ex vivo, or in vivo. For example, the virus vectors may be introduced into cultured cells and the expressed gene product isolated therefrom.

It will be understood by those skilled in the art that the heterologous nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Further, regulated expression of the heterologous nucleic acid(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The virus vectors according to the present invention provide a means for delivering heterologous nucleic acids into a broad range of cells, including dividing and non-dividing cells. The virus vectors can be employed to deliver a nucleic acid of interest to a cell in vitro, e.g., to produce a polypeptide in vitro or for ex vivo gene therapy. The virus vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the present invention can be employed to deliver a heterologous nucleic acid encoding a polypeptide or functional RNA to treat and/or prevent any disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (ß-globin), anemia (erythropoietin) and other blood disorders, Alzheimer's disease (GDF; neprilysin), multiple sclerosis (ß-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic disorders, congenital emphysema (α1-antitrypsin), Lesch-Nyhan Syndrome (hypoxanthine guanine phosphoribosyl transferase), Niemann-Pick disease (sphingomyelinase), Tay-Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., I1C), serca2a, zinc finger proteins that regulate the phospholamban gene, Barkct, β2-adrenergic receptor, β2-adrenergic receptor kinase (BARK), phosphoinositide-3 kinase (PI3 kinase), S100-X¹, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

The invention can also be used to produce induced pluripotent stem cells (iPS). For example, a virus vector of the invention can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like. Nucleic acids encoding factors associated with stem cells are known in the art. Nonlimiting examples of such factors associated with stem cells and pluripotency include Oct-3/4, the SOX family (e.g., SOX¹, SOX², SOX³ and/or SOX¹⁵), the Klf family (e.g., Klf1, Klf2, Klf4 and/or Klf5), the Myc family (e.g., C-myc, L-myc and/or N-myc), NANOG and/or LIN28.

The invention can also be practiced to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome [β-glucuronidase], Hurler Syndrome [α-L-iduronidase], Scheie Syndrome [α-L-iduronidase], Hurler-Scheie Syndrome [α-L-iduronidase], Hunter's Syndrome [iduronate sulfatase], Sanfilippo Syndrome A [heparan sulfamidase], B [N-acetylglucosaminidase], C [acetyl-CoA:α-glucosaminide acetyltransferase], D [N-acetylglucosamine 6-sulfatase], Morquio Syndrome A [galactose-6-sulfate sulfatase], B [β-galactosidase], Maroteaux-Lamy Syndrome [N-acetylgalactosamine-4-sulfatase], etc.), Fabry disease (α-galactosidase), Gaucher's disease (glucocerebrosidase), or a glycogen storage disorder (e.g., Pompe disease; lysosomal acid α-glucosidase).

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors according to the present invention permit the treatment and/or prevention of genetic diseases.

The virus vectors according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods.

In addition, virus vectors according to the instant invention find use in diagnostic and screening methods, whereby a nucleic acid of interest is transiently or stably expressed in a cell culture system, or alternatively, a transgenic animal model.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the virus vectors of the present invention may be used to produce an immune response in a subject. According to this embodiment, a virus vector comprising a heterologous nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the virus vector may be administered to a cell ex vivo and the altered cell is administered to the subject. The virus vector comprising the heterologous nucleic acid is introduced into the cell, and the cell is administered to the subject, where the heterologous nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In particular embodiments, the virus vector or cell comprising the heterologous nucleic acid can be administered in an immunogenically effective amount, as described below.

The virus vectors of the present invention can also be administered for cancer immunotherapy by administration of a virus vector expressing one or more cancer cell antigens (or an immunologically similar molecule) or any other immunogen that produces an immune response against a cancer cell. To illustrate, an immune response can be produced against a cancer cell antigen in a subject by administering a virus vector comprising a heterologous nucleic acid encoding the cancer cell antigen, for example to treat a patient with cancer and/or to prevent cancer from developing in the subject. The virus vector may be administered to a subject in vivo or by using ex vivo methods, as described herein. Alternatively, the cancer antigen can be expressed as part of the virus capsid or be otherwise associated with the virus capsid (e.g., as described above).

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with a virus vector expressing a cancer cell antigen according to the instant invention. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

### Subjects, Pharmaceutical Formulations, and Modes of Administration.

Virus vectors and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles, adults and geriatric subjects.

In representative embodiments, the subject is “in need of” the methods of the invention.

In particular embodiments, the present invention provides a pharmaceutical composition comprising a virus vector and/or capsid and/or capsid protein and/or virus particle of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, optionally at least about 10⁵ infectious units are introduced to the cell.

The cell(s) into which the virus vector is introduced can be of any type, including but not limited to neural cells (including cells of the peripheral and central nervous systems, in particular, brain cells such as neurons and oligodendricytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

In some embodiments, the virus vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

Thus, the present invention provides a method of administering a nucleic acid to a cell, the method comprising contacting the cell with the virus vector, virus particle and/or composition of this invention.

A further aspect of the invention is a method of administering the virus vector, virus particle and/or virus capsid of this invention to a subject. Thus, the present invention also provides a method of delivering a nucleic acid to a subject, comprising administering to the subject a virus particle, virus vector and/or composition of this invention. Administration of the virus vectors, virus particles and/or capsids according to the present invention to a human subject or an animal in need thereof can be by any means known in the art. Optionally, the virus vector, virus particle and/or capsid is delivered in a treatment effective or prevention effective dose in a pharmaceutically acceptable carrier.

The virus vectors and/or capsids of the invention can further be administered to elicit an immunogenic response (e.g., as a vaccine). Typically, immunogenic compositions of the present invention comprise an immunogenically effective amount of virus vector and/or capsid in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the virus vector and/or capsid to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10³, 10¹⁴, 10¹⁵ transducing units, optionally about 10⁸-10¹³ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The virus vector and/or capsid can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the virus vector and/or capsid is administered to a limb (arm and/or leg) of a subject (e.g., a subject with muscular dystrophy such as DMD) by limb perfusion, optionally isolated limb perfusion (e.g., by intravenous or intra-articular administration). In embodiments of the invention, the virus vectors and/or capsids of the invention can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the vector to cross the endothelial cell barrier. In particular embodiments, the viral vectors and/or capsids of the invention can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The virus vector and/or capsid can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Delivery to a target tissue can also be achieved by delivering a depot comprising the virus vector and/or capsid. In representative embodiments, a depot comprising the virus vector and/or capsid is implanted into skeletal, cardiac and/or diaphragm muscle tissue or the tissue can be contacted with a film or other matrix comprising the virus vector and/or capsid. Such implantable matrices or substrates are described in U.S. Pat. No. 7,201,898.

In particular embodiments, a virus vector and/or virus capsid according to the present invention is administered to skeletal muscle, diaphragm muscle and/or cardiac muscle (e.g., to treat and/or prevent muscular dystrophy, heart disease [for example, PAD or congestive heart failure]).

In representative embodiments, the invention is used to treat and/or prevent disorders of skeletal, cardiac and/or diaphragm muscle.

In a representative embodiment, the invention provides a method of treating and/or preventing muscular dystrophy in a subject in need thereof, the method comprising: administering a treatment or prevention effective amount of a virus vector of the invention to a mammalian subject, wherein the virus vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the virus vector can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional RNA (e.g., RNAi, microRNA, antisense RNA) that normally circulates in the blood or for systemic delivery to other tissues to treat and/or prevent a disorder (e.g., a metabolic disorder, such as diabetes [e.g., insulin], hemophilia [e.g., Factor IX or Factor VIII], a mucopolysaccharide disorder [e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.] or a lysosomal storage disorder such as Gaucher's disease [glucocerebrosidase] or Fabry disease [a-galactosidase A] or a glycogen storage disorder such as Pompe disease [lysosomal acid a glucosidase]). Other suitable proteins for treating and/or preventing metabolic disorders are described herein. The use of muscle as a platform to express a nucleic acid of interest is described in U.S. Patent publication US 2002/0192189.

Thus, as one aspect, the invention further encompasses a method of treating and/or preventing a metabolic disorder in a subject in need thereof, the method comprising: administering a treatment or prevention effective amount of a virus vector of the invention to skeletal muscle of a subject, wherein the virus vector comprises a heterologous nucleic acid encoding a polypeptide, wherein the metabolic disorder is a result of a deficiency and/or defect in the polypeptide. Illustrative metabolic disorders and heterologous nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering virus vectors to skeletal muscle is described in more detail herein.

The invention can also be practiced to produce antisense RNA, RNAi or other functional RNA (e.g., a ribozyme) for systemic delivery.

The invention also provides a method of treating and/or preventing congenital heart failure or PAD in a subject in need thereof, the method comprising administering a treatment or prevention effective amount of a virus vector of the invention to a mammalian subject, wherein the virus vector comprises a heterologous nucleic acid encoding, for example, a sarcoplasmic endoreticulum Ca²⁺-ATPase (SERCA2a), an angiogenic factor, phosphatase inhibitor I (I-1) and fragments thereof (e.g., I1C), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (βARKct), inhibitor 1 of protein phosphatase 1 and fragments thereof (e.g., I1C), S100-A1, parvalbumin, adenylyl cyclase type 6, a molecule that effects G-protein coupled receptor kinase type 2 knockdown such as a truncated constitutively active bARKct, Pim-1, PGC-1α, SOD-1, SOD-2, EC-SOD, kallikrein, HIF, thymosin-β4, mir-1, mir-133, mir-206, mir-208 and/or mir-26a.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1).

The virus vectors and/or virus capsids disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

The virus vectors and virus capsids can be administered to tissues of the CNS (e.g., brain, eye) and may advantageously result in broader distribution of the virus vector or capsid than would be observed in the absence of the present invention.

In particular embodiments, the delivery vectors of the invention may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a delivery vector encoding one or more neurotrophic factors.

Age-related macular degeneration involves both angiogenesis and retinal degeneration. This disorder can be treated by administering the inventive deliver vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using a delivery vector of the invention to treat a pituitary tumor. According to this embodiment, the delivery vector encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

In particular embodiments, the vector can comprise a secretory signal as described in U.S. Pat. No. 7,071,172.

In representative embodiments of the invention, the virus vector and/or virus capsid is administered to the CNS (e.g., to the brain or to the eye). The virus vector and/or capsid may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The virus vector and/or capsid may also be administered to different regions of the eye such as the retina, cornea and/or optic nerve.

The virus vector and/or capsid may be delivered into the cerebrospinal fluid (e.g., by lumbar puncture) for more disperse administration of the delivery vector. The virus vector and/or capsid may further be administered intravascularly to the CNS in situations in which the blood-brain barrier has been perturbed (e.g., brain tumor or cerebral infarct).

The virus vector and/or capsid can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the virus vector and/or capsid is administered in a liquid formulation by direct injection (e.g., stereotactic injection) to the desired region or compartment in the CNS. In other embodiments, the virus vector and/or capsid may be provided by topical application to the desired region or by intra-nasal administration of an aerosol formulation. Administration to the eye, may be by topical application of liquid droplets. As a further alternative, the virus vector and/or capsid may be administered as a solid, slow-release formulation (see, e.g., U.S. Pat. No. 7,201,898).

In yet additional embodiments, the virus vector can used for retrograde transport to treat and/or prevent diseases and disorders involving motor neurons (e.g., amyotrophic lateral sclerosis (ALS); spinal muscular atrophy (SMA), etc.). For example, the virus vector can be delivered to muscle tissue from which it can migrate into neurons.

Having described the present invention, the same will be explained in greater detail in the following examples, which are included herein for illustration purposes only, and which are not intended to be limiting to the invention.

### EXAMPLES

### Example 1. Combinatorial Engineering and Selection of Antibody-Evading AAV Vectors (AAV1e Clones 1-26)

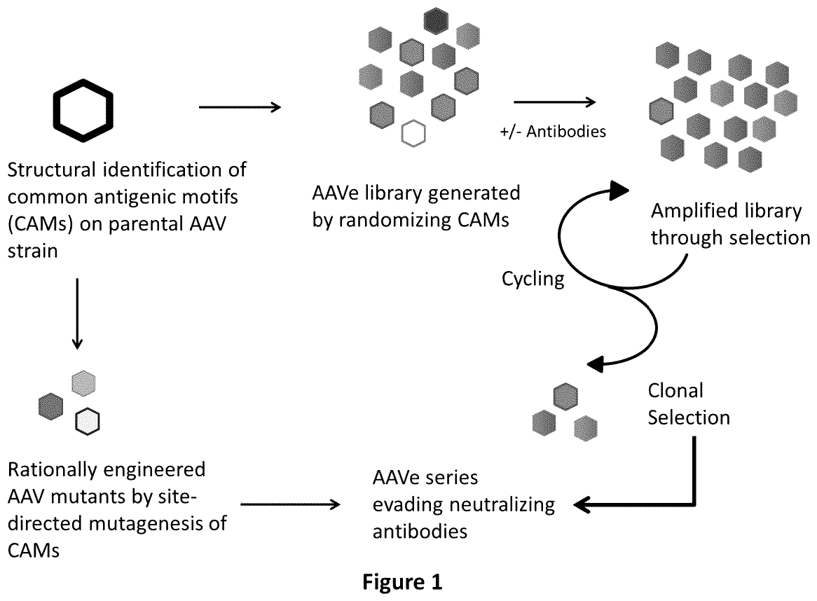

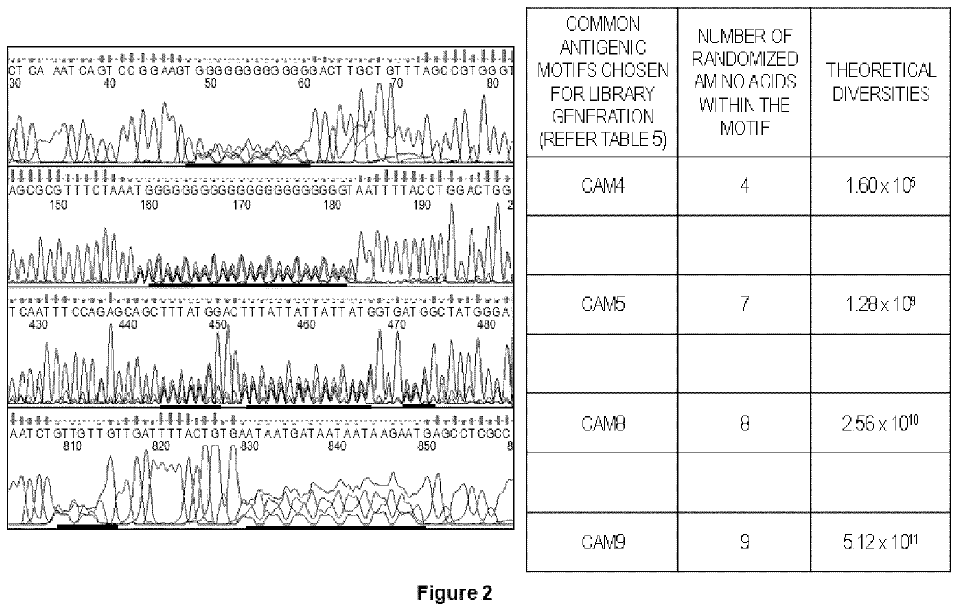

The method for generating antibody evading AAVe mutants is as follows. A general schematic description of the approach is provided in FIG. 1. As an example, the first step involves identification of conformational 3D antigenic epitopes on the AAV capsid surface from cryo-electron microscopy. Selected residues within antigenic motifs are then subjected to mutagenesis using degenerate primers with each codon substituted by nucleotides NNK and gene fragments combined together by Gibson assembly and/or multistep PCR. Capsid-encoding genes containing a degenerate library of mutated antigenic motifs are cloned into a wild type AAV genome to replace the original Cap encoding DNA sequence yielding a plasmid library. Plasmid libraries are then transfected into 293 producer cell lines to generate AAV capsid libraries, which can then be subjected to selection. Successful generation of AAV libraries is confirmed via DNA sequencing (FIG. 2). In order to select for new AAV strains that can escape neutralizing antibodies (NAbs), AAV libraries are subjected to multiple rounds of infection in specific cells or tissues in the presence of a helper virus such as adenovirus with or without different monoclonal antibodies, polyclonal antibodies or serum containing anti-AAV antibodies. Cell lysates harvested from at least one round of successful infection and replication are sequenced to identify single AAV isolates escaping antibody neutralization.

As a nonlimiting specific example, common antigenic motifs on the AAV1 capsid protein (VP1) were subjected to mutagenesis as described above. The degenerate libraries were then subjected to infection in endothelial cells in culture for five cycles of infection and replication. Cells were infected with AAV1 libraries on day 0, infected with adenovirus at day 1 and cell lysates as well as supernatant were obtained at day 7 post-infection for repeating the cycle of infection and replication. This procedure was repeated five times following which, fifteen to twenty isolated clones from each library were subjected to DNA sequence analysis (FIG. 2). Each unique sequence was labeled as AAV1e (#number), where the number depicts the specific clonal isolate (Tables 6.1 to 6.4).

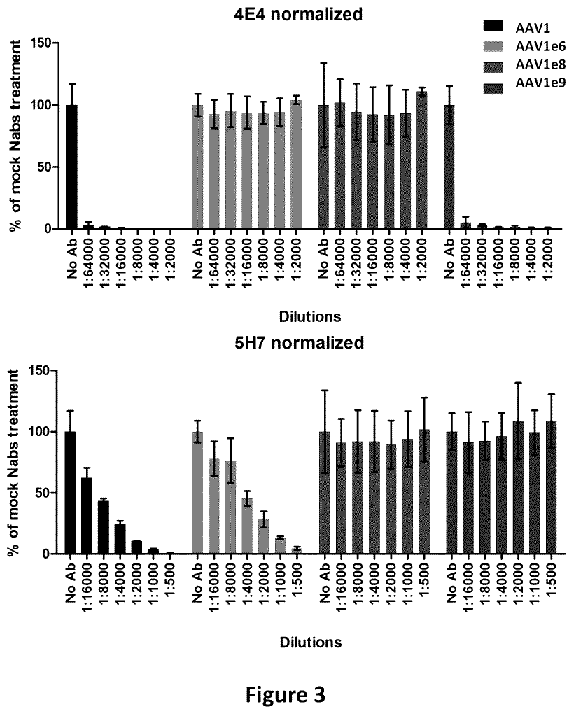

For validation of AAV1e mutants and their ability to escape neutralization, AAV1 neutralizing antibodies, 4E4 (FIG. 3 top) and 5H7 (FIG. 3 bottom) were serially diluted in DMEM+5% FBS on a 96 well plate. AAV1 and AAV1e clones packaging a CBA-Luc cassette (5e7vg/well) were added and incubated with antibody on a 96 well plate for 30 min at room temperature. 293 cells (4e5 cells/well) were added into the virus+antibody mix and incubated at 37° C., 5% CO₂ incubator for 48h. Final volume of antibody, virus and cell mixture is 100 ul. Medium was then discarded from individual wells and replaced with 25 ul of passive lysis buffer. After 30 min incubation at room temperature, 25 ul of luciferin was added and reporter transgene expression (transduction efficiency) was assayed using a Victor3 illuminometer.

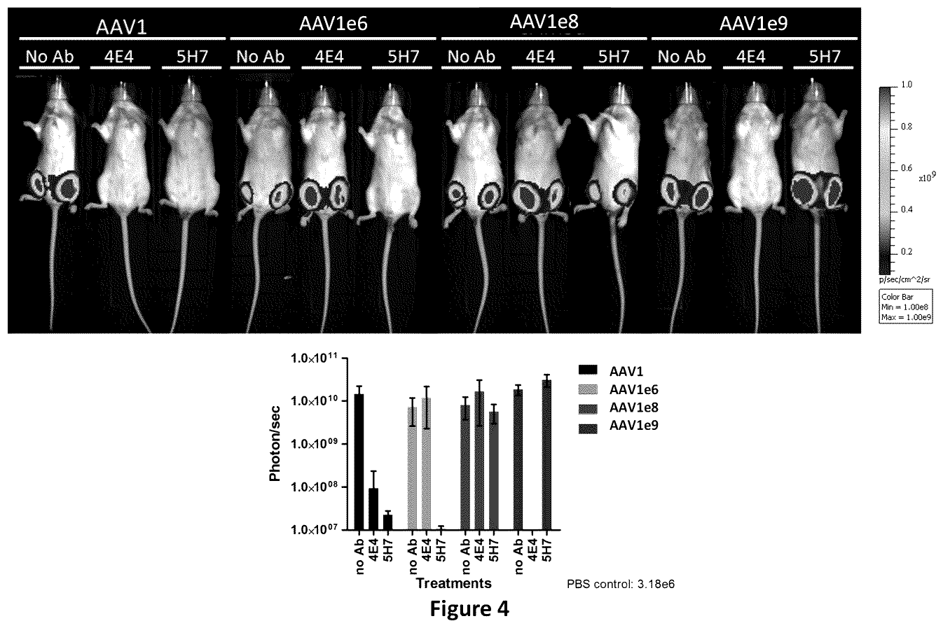

For validation of AAV1e mutants in mouse models in vivo (FIG. 4), a dose of 1e9vg/u1 was pre-incubated with neutralizing antibodies 4E4 (1:500) or 5H7 (1:10), or with PBS for 1 h at room temperature. Each mouse (6-8 weeks old, BALB/c, female) was injected with 20 ul of the virus and antibody mixture into each gastrocnemius muscle in the hind leg (2e10 vg/leg) through an intramuscular injection.

Mice were anesthetized with isoflurane and injected with 150 ul of RediJect D-lucifercin intraperitoneally (IP) at different time intervals for live animal imaging and luciferase reporter expression. Luciferase activities of each mouse were imaged 1 min after the injection using a Xenogen IVIS Lumina® system. Live animal luciferase imaging was performed at 1 week and 4 weeks post-injection and luciferase activities quantified to determine differences in the ability of AAV1e clones to evade neutralizing antibodies (FIG. 4).

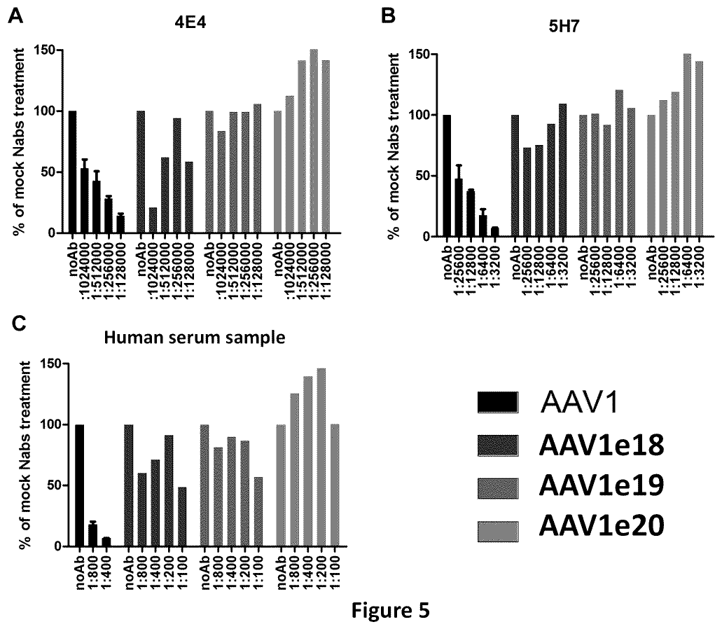

For further enhancement of antibody evading properties, mutations discovered in AAV1e clones were combined on capsids to generate new AAV1e strains (clones 18 through 20). These clones were subjected to in vitro transduction assays in order to determine their ability to evade antibody neutralization. Clones AAV1e18-20 demonstrated the ability to escape both monoclonal antibodies against AAV1 or human serum sample containing polyclonal antibodies (FIG. 5).

### Example 2. Rational Engineering of Antibody-Evading AAV Vectors (AAV1e Series 27-36, AAV9e1, and AAV9e2)

Current WT AAV vectors are likely to have pre-existing antibodies targeted against the capsid surface, which prevents efficient transduction. Vectors of this invention overcome these limitations.

This invention provides AAV antibody escape variants that retain transduction efficiency. They are engineered to overcome pre-existing antibody responses based on capsid interaction sites and capsid-antibody structures, and can be further engineered to target specific tissues.

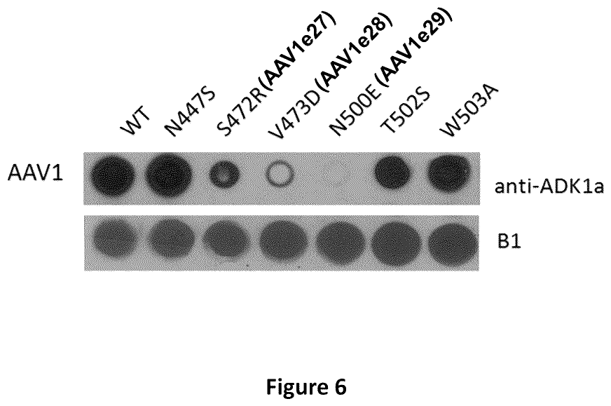

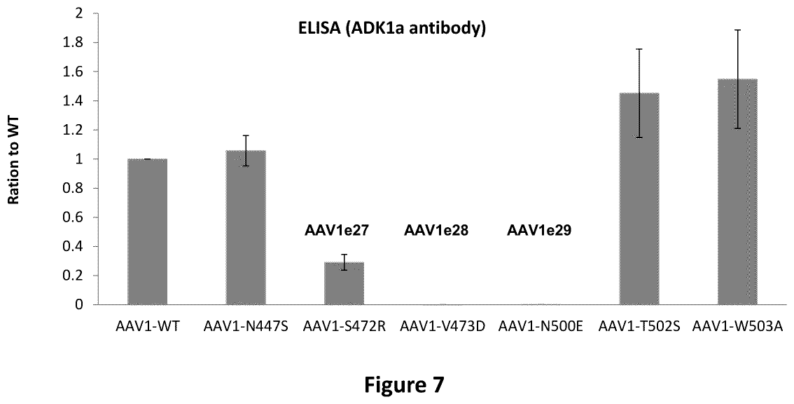

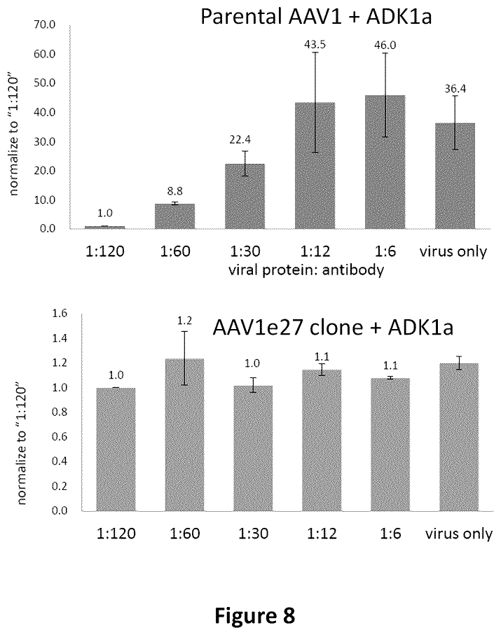

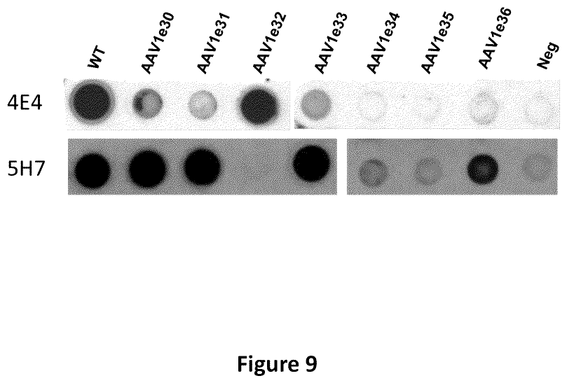

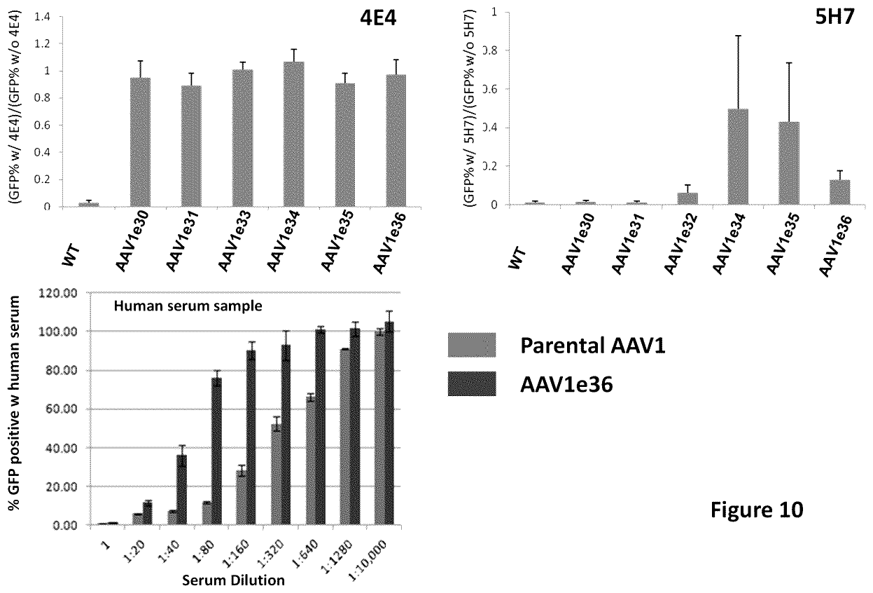

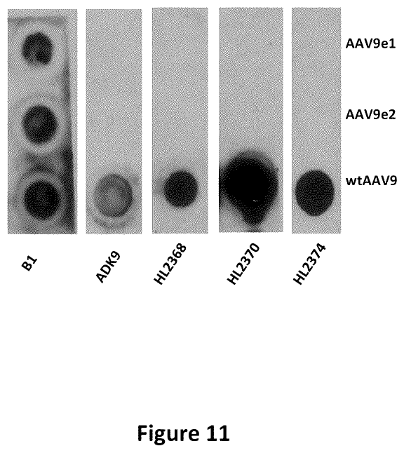

We have designed AAV1 as well as AAV9 variants to escape anti-AAV capsid monoclonal binding and host antibody neutralization based on antigenic epitope information attained from 3D structural characterization of AAV capsids, receptor binding sites, and AAV-antibody complex structures determined by cryo-electron microscopy and image reconstruction. These vectors contain amino acid alterations in variable regions of the capsid, which have been established as common antigenic motifs (CAMs; Table 5). Amino acid residues within these CAMs have been modified to generate novel AAV strains that can escape neutralizing antibodies (AAVe series) in order to overcome pre-existing immunity (Tables 7 and 8), which has been reported to be detrimental to AAV transduction efficacy in pre-clinical animal studies and in human clinical trials. We have tested the mutants described herein and observe, using biochemical approaches including dot blots and ELISA (FIGS. 6, 7, 9 and 11), that these mutants escape recognition by antibodies targeted at the parental capsid, escape neutralization in the presence of anti-capsid antibodies (FIGS. 8 and 10), and display significantly reduced recognition by sera obtained from patients participating in a clinical trial utilizing AAV1 as the gene delivery vector (FIG. 10).

TABLE 5
Representative list of common antigenic motifs (CAMs) found on different AAV serotypes and
isolates (respective VP1 numbering of residues and different amino acid residues is shown).

| | | CAM3 | | | CAM5 |
|---|---|---|---|---|---|
| | CAM1 (SEQ ID NO:) | (SEQ ID NO:) | CAM4-1 (SEQ ID NO:) | CAM4-2 | (SEQ ID NO:) |
| AAV1 | 262-SASTGAS-268 (303) | 370-VFMIPQYGYL- | 451-NQSGSAQNK-459 (305) | 472-SV-473 | 493-KTDNNNSN- |
| | | 379 (304) | | | 500 (306) |
| AAV2 | 262-SQSGAS-267 (311) | 369-VFMVPQYGYL- | 450-TPSGTTTQS-458 (313) | 471-RD-472 | 492-SADNNNSE- |
| | | 378 (312) | | | 499 (314) |
| AAV3 | 262-SQSGAS-267 (319) | 369-VFMVPQYGYL- | 451-TTSGTTNQS-459 (321) | 472-SL-473 | 493-ANDNNNSN- |
| | | 378 (320) | | | 500 (322) |
| AAV4 | 253-RLGESLQS-260 (327) | 360-VFMVPQYGYC- | 445-GTTLNAGTA-453 (329) | 466-SN-467 | 487- |
| | | 369 (328) | | | ANQNYKIPATGS- |
| | | | | | 498 (330 |
| AAV5 | 249-EIKSGSVDGS- | 360-VFTLPQYGYA- | 440-STNNTGGVQ-448 (337) | 458-AN-459 | 479-SGVNRAS- |
| | 258 (335) | 369 (336) | | | 485 (338) |
| AAV6 | 262-SASTGAS-268 (343) | 370-VFMIPQYGYL- | 451-NQSGSAQNK-459 (345) | 472-SV-473 | 493-KTDNNNSN- |
| | | 379 (344) | | | 500 (346) |
| AAV7 | 263-SETAGST-269 (351) | 371-VFMIPQYGYL- | 453-NPGGTAGNR-461 (353) | 474-AE-475 | 495-LDQNNNSN- |
| | | 380 (352) | | | 502 (354) |
| AAV8 | 263-NGTSGGAT-270 (359) | 372-VFMIPQYGYL- | 453-TTGGTANTQ-461 (361) | 474-AN-475 | 495-TGQNNNSN- |
| | | 381 (360) | | | 502 (362) |
| AAV9 | 262-NSTSGGSS-269 (367) | 371-VFMIPQYGYL- | 451-INGSGQNQQ-459 (369) | 472-AV-473 | 493-VTQNNNSE- |
| | | 380 (368) | | | 500 (370) |
| AAVrh8 | 262-NGTSGGST-269 (375) | 371-VFMVPQYGYL- | 451-QTTGTGGTQ-459 (377) | 472-AN-473 | 493-TNQNNNSN- |
| | | 380 (376) | | | 500 (378) |
| AAVrh10 | 263-NGTSGGST-270 (383) | 372-VFMIPQYGYL- | 453-STGGTAGTQ-461 (385) | 474-SA-475 | 495-LSQNNNSN- |
| | | 381 (384) | | | 502 (386) |
| AAV10 | 263-NGTSGGST-270 (391) | 372-VFMIPQYGYL- | 453-STGGTQGTQ-461 (393) | 474-SA-475 | 495-LSQNNNSN- |
| | | 381 (392) | | | 502 (394) |
| AAV11 | 253-RLGTTSSS-260 (399) | 360-VFMVPQYGYC- | 444-GETLNQGNA-452 (401) | 465-AF-466 | 486- |
| | | 369 (400) | | | ASQNYKIPASGG- |
| | | | | | 497 (402) |
| AAV12 | 262-RIGTTANS-269 (407) | 369-VFMVPQYGYC- | 453-GNSLNQGTA-461 (409) | 474-AY-475 | 495- |
| | | 378 (408) | | | ANQNYKIPASGG- |
| | | | | | 506 (410) |
| AAVrh32.33 | 253-RLGTTSNS-260 (415) | 360-VFMVPQYGYC- | 444-GETLNQGNA-452 (417) | 465-AF-466 | 486- |
| | | 369 (416) | | | ASQNYKIPASGG- |
| | | | | | 497 (418) |
| Bovine AAV | 255-RLGSSNAS-262 (423) | 362-VFMVPQYGYC- | 447-GGTLNQGNS-455 (425) | 468-SG-469 | 489- |
| | | 371 (424) | | | ASQNYKIPQGRN- |
| | | | | | 500 (426) |
| Avian AAV | 265-RIQGPSGG-272 (431) | 375-IYTIPQYGYC- | 454-VSQAGSSGR-462 (433) | 475-AA-476 | 496- |
| | | 384 (432) | | | ASNITKNNVFSV- |
| | | | | | 507 (434) |
| | | CAM7 | | | CAM9-2 |
| | CAM6 (SEQ ID NO:) | (SEQ ID NO:) | CAM8 (SEQ ID NO:) | CAM9-1 | (SEQ ID NO:) |
| AAV1 | 528-KDDEDKF-534 (307) | 547-SAGASN- | 588-STDPATGDVH-597 (309) | 709-AN-710 | 716-DNNGLYT- |
| | | 552 (308) | | | 722 (310) |
| AAV2 | 527-KDDEEKF-533 (315) | 546-GSEKTN- | 587-NRQAATADVN-596 (317) | 708-VN-709 | 715-DTNGVYS- |
| | | 551 (316) | | | 721 (318) |
| AAV3 | 528-KDDEEKF-534 (323) | 547-GTTASN- | 588-NTAPTTGTVN-597 (325) | 709-VN-710 | 716-DTNGVYS- |
| | | 552 (324) | | | 722 (326) |
| AAV4 | 527-GPADSKF-533 (331) | 545-QNGNTA- | 586-SNLPTVDRLT-595 (333) | 707-NS-708 | 714-DAAGKYT- |
| | | 560 (332) | | | 720 (334) |
| AAV5 | 515-LQGSNTY-521 (339) | 534-ANPGTTAT- | 577-TTAPATGTYN-586 (341) | 697-QF-698 | 704-DSTGEYR- |
| | | 541 (340) | | | 710 (342) |
| AAV6 | 528-KDDKDKF-534 (347) | 547-SAGASN- | 588-STDPATGDVH-597 (349) | 709-AN-710 | 716-DNNGLYT- |
| | | 552 (348) | | | 722 (350) |
| AAV7 | 530-KDDEDRF-536 (355) | 549-GATNKT- | 589-NTAAQTQVVN-598 (357) | 710-TG-711 | 717-DSQGVYS- |
| | | 554 (356) | | | 723 (358) |
| AAV8 | 530-KDDEERF-536 (363) | 549-NAARDN- | 590-NTAPQIGTVNS-600 (365) | 711-TS-712 | 718-NTEGVYS- |
| | | 554 (364) | | | 724 (366) |
| AAV9 | 528-KEGEDRF-534 (371) | 547-GTGRDN- | 588-QAQAQTGWVQ-597 (373) | 709-NN-710 | 716-NTEGVYS- |
| | | 552 (372) | | | 722 (374) |
| AAVrh8 | 528-KDDDDRF-534 (379) | 547-GAGNDG- | 588-NTQAQTGLVH-597 (381) | 709-TN-710 | 716-NTEGVYS- |
| | | 552 (380) | | | 722 (382) |
| AAVrh10 | 530-KDDEERF-536 (387) | 549-GAGKDN- | 590-NAAPIVGAVN-599 (389) | 711-TN-712 | 718-NTDGTYS- |
| | | 554 (388) | | | 724 (390) |
| AAV10 | 530-KDDEERF-536 (395) | 549-GAGRDN- | 590-NTGPIVGNVN-599 (397) | 711-TN-712 | 718-NTEGTYS- |
| | | 554 (396) | | | 724 (398) |
| AAV11 | 526-GPSDGDF-532 (403) | 544-VTGNTT- | 585-TTAPITGNVT-594 (405) | 706-SS-707 | 713-DTTGKYT- |
| | | 549 (404) | | | 719 (406) |
| AAV12 | 535-GAGDSDF-541 (411) | 553-PSGNTT- | 594-TTAPHIANLD-603 (413) | 715-NS-716 | 722-DNAGNYH- |
| | | 558 (412) | | | 728 (414) |
| AAVrh32.33 | 526-GPSDGDF-532 (419) | 544-VTGNTT- | 585-TTAPITGNVT-594 (421) | 706-SS-707 | 713-DTTGKYT- |
| | | 549 (420) | | | 719 (422) |
| Bovine AAV | 529-ANDATDF-535 (427) | 547-ITGNTT- | 588-TTVPTVDDVD-597 (429) | 709-DS-710 | 716-DNAGAYK- |
| | | 552 (428) | | | 722 (430) |
| Avian AAV | 533-FSGEPDR-539 (435) | 552-VYDQTTAT- | 595-VTPGTRAAVN-604 (437) | 716-AD-717 | 723-SDTGSYS- |
| | | 559 (436) | | | 729 (438) |

TABLE 6.1
AAV1e1-7.

| Nab Evading | Novel amino acid | |
|---|---|---|
| AAV1e | sequence identified in | |
| strains | corresponding AAV1e isolate | Frequency |
| AAV1e1 | 456-QVRG-459 (SEQ ID NO: 22) | 10/19 |
| AAV1e2 | 456-GRGG-459 (SEQ ID NO: 24) | 1/19 |
| AAV1e3 | 456-SGGR-459 (SEQ ID NO: 25) | 1/19 |
| AAV1e4 | 456-ERPR-459 (SEQ ID NO: 23) | 1/19 |
| AAV1e5 | 456-SERR-459 (SEQ ID NO: 26) | 1/19 |
| AAV1e6 | 456-LRGG-459 (SEQ ID NO: 27) | 1/19 |
| AAV1e7 | 456-ERPR-459 (SEQ ID NO: 23), | 4/19 |
| | D595N | |

List of novel neutralizing antibody evading AAV1e strains isolated after screening and selection. Each strain is labeled as AAV1eN, where N is the strain number. Amino acid residues that were selected by this approach within the different common antigenic motifs are listed with VP1 capsid protein numbering. In each case, 15-25 clones isolated from the library screen were sent for sequence analysis the relative frequencies of each strain is also listed.

TABLE 6.2
AAV1e8-16.

| Nab | | |
|---|---|---|
| Evading | Novel amino | |
| AAV1e | acid sequence identified in | Fre- |
| strains | corresponding AAV1e isolate | quency |
| AAV1e8 | 493-PGGNATR-499 (SEQ ID NO: 30) | 15/15 |
| AAV1e9 | 588-TADHDTKGVL-597 (SEQ ID NO: 32) | 15/24 |
| AAV1e10 | 588-VVDPDKKGVL-597 (SEQ ID NO: 33) | 1/24 |
| AAVle11 | 588-AKDTGPLNVM-597 (SEQ ID NO: 34) | 2/24 |
| AAV1e12 | 588-QTDAKDNGVQ-597 (SEQ ID NO: 35) | 1/24 |
| AAV1e13 | 588-DKDPWLNDVI-597 (SEQ ID NO: 36) | 1/24 |
| AAV1e14 | 588-TRDGSTESVL-597 (SEQ ID NO: 37) | 2/24 |
| AAV1e15 | 588-VIDPDQKGVL-597 (SEQ ID NO: 38) | 1/24 |
| AAV1e16 | 588-VNDMSNYMVH-597 (SEQ ID NO: 39) | 1/24 |

List of novel neutralizing antibody evading AAV1e strains isolated after screening and selection (Cont'd)

TABLE 6.3
(AAV1e17-20).

| Nab Evading | |
|---|---|
| AAV1e strains | |
| (Combination | Amino acid sequences combined |
| mutant strains) | by rational mutagenesis |
| AAV1e17 | (456-LRGG-459, SEQ ID NO: 27) + |
| | (493-PGGNATR-499, SEQ ID NO: 30) |
| AAV1e18 | (456-LRGG-459, SEQ ID NO: 27) + |
| | (588-TADHDTKGVL-597, SEQ ID NO: 32) |
| AAV1e19 | (493-PGGNATR-499, SEQ ID NO: 30) + |
| | (588-TADHDTKGVL-597, SEQ ID NO: 32) |
| AAV1e20 | (456-LRGG-459, SEQ ID NO: 27) + |
| | (493-PGGNATR-499, SEQ ID NO: 30) + |
| | (588-TADHDTKGVL-597, SEQ ID NO: 32) |

List of novel neutralizing antibody evading AAV1e generated by making various rationally engineered permutations and combinations of amino acid sequences derived from AAV1e6, AAV and AAV1e9.

TABLE 6.4
AAV1e21-26.

| Nab Evading | | |
|---|---|---|
| AAV1e strains | | |
| engineered | Novel amino acid sequence | |
| using AAV1e8 | identified in | Fre- |
| as a template | corresponding AAV1e isolate | quency |
| AAV1e21 | 588-CNDEMQVQVN-597 (SEQ ID | 2/9 |
| | NO: 297) | |
| AAV1e22 | 588-SPDIVYADVC-597 (SEQ ID | 1/9 |
| | NO: 298) | |
| AAV1e23 | 588-LDDCHNIDVN-597 (SEQ ID | 1/9 |
| | NO: 299) | |
| AAV1e24 | 588-SCDCVTNSVS-597 (SEQ ID | 1/9 |
| | NO: 300) | |
| AAV1e25 | 588-TVDSNPYEVN-597 (SEQ ID | 1/9 |
| | NO: 301) | |
| AAV1e26 | 588-GDDHPNPDVL-597 (SEQ ID | 1/9 |
| | NO: 302) | |

List of novel neutralizing antibody evading AAV1e strains isolated after screening and selection (Cont'd.) These novel AAV1e strains contain new sequences listed below in addition to the AAV1e8 sequence 493-PGGNATR-499. Briefly, an AAV1e capsid library was generated using AAV as the template capsid and randomizing common antigenic motif CAM8 (residues 588-597). These were subjected similar screening and isolation protocols to obtain different novel AAV1e isolates.

TABLE 7
AAV1e27-36.

| Nab Evading | Site-specific amino acid mutations |
|---|---|
| AAV1e strains | generated by rational mutagenesis |
| AAV1e27 | S472R |
| AAV1e28 | V473D |
| AAV1e29 | N500E |
| AAV1e30 | A456T + Q457T + N458Q + K459S |
| AAV1e31 | T492S + K493A |
| AAV1e32 | S586R + S587G + S588N + T589R |
| AAV1e33 | A456T + Q457T + N458Q + K459S + T492S + K493A |
| AAV1e34 | A456T + Q457T + N458Q + K459S + S586R + S587G + |
| | S588N + T589R |
| AAV1e35 | T492S + K493A + S586R + S587G + S588N + T589R |
| AAV1e36 | A456T + Q457T + N458Q + K459S + T492S + K493A + |
| | S586R + S587G + S588N + T589R |

List of novel neutralizing antibody evading AAV1e strains generated by making various rationally determined, site-specific mutations on the AAV capsid protein. Single mutants and multiple site mutants are shown.

TABLE 8
AAV9e1 & AAV9e2.

| Antibody Evading | Site-specific amino acid mutations |
|---|---|
| AAV1e strains | generated by rational mutagenesis |
| AAV9e1 | S454V + Q456V |
| AAV9e2 | I451Q + G453Q + Q456S + N457A + N459 insertion |

Proof of concept studies establishing the rational design of novel neutralizing antibody evading AAV9e strains. Table lists the different site-specific point mutations made in AAV9 by rational mutagenesis.

### Example 3. Structure-Based Iterative Evolution of Antigenically Advanced AAV Variants for Therapeutic Gene Transfer

Cells, viruses and antibodies. HEK293 and MB114 cells were maintained in Dulbecco's modified Eagle's medium (DMEM) supplemented with 10% fetal bovine serum (FBS) (ThermoFisher, Waltham, MA), 100 units/ml of penicillin and 10 ng/ml of streptomycin (P/S) (ThermoFisher, Waltham, MA) in 5% CO₂ at 37° C. Murine adenovirus 1 (MAV-1) was purchased from American Type Culture Collection (ATCC, Mannassas, VA) and amplified by infecting MB114 cells at a multiplicity of infection (MOI) of 1. At day 6 post-infection (approximately 50% cytopathic effect (CPE)), media containing progeny MAV-1 viruses were harvested and centrifuged at 3000 g for 5 min, and the supernatant stored at −80° C. for subsequent evolution studies. Mouse anti-AAV1 monoclonal antibodies ADK1a, 4E4 and 5H7 have been described previously. De-identified and naïve human serum samples were purchased from Valley Biomedical, Winchester, VA. Naïve serum from rhesus macaques was from the Yerkes National Primate Center. Antisera against AAV1 capsids, generated by immunizing rhesus macaques intramuscularly (I.M.) with AAV1 capsids was from the Oregon National Primate Center. All mouse, human and non-human primate serum used in this study were heat inactivated at 55° C. for 15 min prior to use.

Recombinant AAV production, purification and quantification. Recombinant AAV vectors were produced by transfecting four 150 mm dishes containing HEK293 cells at 70-80% confluence using polyethylenimine (PEI) according to the triple plasmid protocol. Recombinant vectors packaging single stranded genomes encoding firefly luciferase driven by the chicken beta-actin promoter (ssCBA-Luc) or self-complementary green fluorescence protein driven by a hybrid chicken beta-actin promoter (scCBh-GFP) were generated using this method. Subsequent steps involving harvesting of recombinant AAV vectors and downstream purification were carried out as described previously. Recombinant AAV vector titers were determined by quantitative PCR (qPCR) with primers that amplify AAV2 inverted terminal repeat (ITR) regions, 5′-AACATGCTACGCAGAGAGGGAGTGG-3′ (SEQ ID NO:477), 5′-CATGAGACAAGGAACCCCTAGTGATGGAG-3′ (SEQ ID NO:478).

Structural modeling and analysis of AAV antigenic footprints. Antigenic footprints of AAV serotypes 1/6, AAV2, AAV5, AAV8 and AAV9 were determined using previously resolved structures of AAV capsids complexed with different mouse monoclonal antibodies. To restrict diversity and maximize efficiency of AAV library generation, only amino acid residues directly in contact with antibodies were included for analysis. Contact surface residues on each serotype were either aligned by Clustal Omega software or structurally superimposed using PyMOL (Schrödinger, New York City, N.Y.). Structural alignment revealed that antibody footprints from multiple serotypes overlap in close proximity to the 3-fold symmetry axis, around the 5-fold pore and at the 2-fold depression. Of these so-called common antigenic motifs (CAMs), we determined that 12/18 of the antibodies analyzed have direct contact at the 3-fold symmetry supporting the notion that this region is a critical antigenic determinant. For the current study, antigenic footprints for three distinct monoclonal antibodies (4E4, 5H7 and ADK1a) were visualized on the AAV1 capsid (PDB ID: 3ng9) and roadmap images were generated using the RIVEM program.

Generation of AAV capsid libraries. AAV libraries were engineered through saturation mutagenesis of amino acid residues within different antigenic footprints associated with distinct monoclonal antibodies described above. Briefly, for Gibson assembly, twelve oligos with an average length of 70 nucleotides were ordered from IDT (Coralville, IA). Each oligo contains at least 15-20 nt overlapping homology to the neighboring oligos. Three oligos contained degenerate nucleotides (NNK) within genomic regions coding for different antigenic footprints. Plasmid libraries were then generated by in vitro assembly of multiple oligos using High Fidelity Gibson Assembly Mix (NEB, Ipswich, MA) according to manufacturer instructions. The assembled fragments were either PCR amplified for 10 cycles using Phusion HF (NEB, Ipswich, MA) or directly cloned into pTR-AAV1** plasmids between the BspEI and SbfI restriction sites. Plasmid pTR-AAV1** contains genes encoding AAV2 Rep and AAV1 Cap with 2 stop codons at positions 490 and 491 (AAV1 VP1 numbering) introduced by site directed mutagenesis (Agilent, Santa Clara, CA). The entire construct is flanked by AAV2 inverted terminal repeats (ITRs) to enable packaging and replication of pseudotyped AAV1 libraries upon helper virus co-infection. It is noteworthy to mention that the AAV1** capsid gene was incorporated prior to library cloning in order to reduce wild type AAV1 contamination within the different libraries. Ligation reactions were then concentrated and purified by ethanol precipitation. Purified ligation products were electroporated into DH10B electroMax (Invitrogen, Carlsbad, CA) and directly plated on multiple 5245 mm 2 bioassay dishes (Corning, Corning, NY) to avoid bias from bacterial suspension cultures. Plasmid DNA from pTR-AAV1CAM libraries was purified from pooled colonies grown on LB agar plates using a Maxiprep kit (Invitrogen, Carlsbad, CA).

Directed evolution of novel AAV CAM strains. Equal amounts (15 ng each) of each pTR-AAV1CAM library and the Ad helper plasmid, pXX⁶⁸⁰, were transfected onto HEK293 cells at 70-80% confluency on each 150 mm dish using PEI to generate CAM viral libraries. AAV CAM libraries were purified using standard procedures described earlier. MB114 cells were seeded on a 100 mm tissue culture dish overnight to reach 60-70% confluence before inoculation with AAV CAM libraries at an MOI ranging from 1000-10,000. After 24 h post-transduction, MAV-1 was added as helper virus to promote AAV replication. At 6 days post-infection with MAV-1 (50% CPE), the supernatant was harvested and DNase I resistant vector genomes were quantified on day 7. Media containing replicating AAV strains and MAV-1 obtained from each round of infection were then used as inoculum for each subsequent cycle for a total of 5 rounds of evolution. Subsequent iterative rounds of evolution were carried out in a similar fashion with AAV capsid libraries containing different permutations and combinations of newly evolved antigenic footprints.

Identification of newly evolved AAV strains. To analyze sequence diversity of the parental and evolved AAV CAM libraries, DNase I resistant vector genomes were isolated from media and amplified by Q5 polymerase for 10-18 cycles (NEB, Ipswich, MA) using primers, 5′-CCCTACACGACGCTCTTCCGATCTNNNNNcagaactcaaaatcagtcagtccggaagt-3′ (SEQ ID NO:479) and 5′-GACTGGAGTTCAGACGTGTGCTCTTCCGATCNNNNNgccaggtaatgctcccatagc-3′ (SEQ ID NO:480). Illumina MiSeq sequencing adaptor for multiplexing was added through a second round of PCR using Q5 Polymerase with P5 and P7 primers. After each round of PCR, the products were purified using a PureLink PCR Micro Kit (ThermoFisher, Waltham, MA). Quality of the amplicons was verified using a Bioanalyzer (Agilent), and concentrations quantified using a Qubit spectrometer (ThermoFisher, Waltham, MA). Libraries were then prepared for sequencing with a MiSeq 300 Kit v2, following manufacturer instructions, and sequenced on the MiSeq system (Illumina).

Sequencing data analysis. De-multiplexed reads were analyzed via a custom Perl script. Briefly, raw sequencing files were probed for mutagenized regions of interest, and the frequencies of different nucleotide sequences in this region were counted and ranked for each library. Nucleotide sequences were also translated, and these amino acid sequences were similarly counted and ranked. Amino acid sequence frequencies across libraries were then plotted in R.

Isolation of AAV CAM variants for characterization. To characterize selected clones from each library, DNase I resistant vector genomes were isolated from media and amplified by Phusion HF (NEB, Ipswich, MA) using primers flanking the BspEI and SbfI sites. The PCR products were gel purified, sub-cloned into TOPO cloning vectors (ThermoFisher, Waltham, MA) and sent out for standard Sanger sequencing (Eton Bioscience, San Diego, CA). Unique sequences were sub-cloned into an AAV helper plasmid backbone, pXR, using BspEI and SbfI sites. Unique recombinant AAV CAM variants were produced following a standard rAAV production protocol as described above.

In vitro antibody and serum neutralization assays. Twenty-five microliters of antibodies or antisera (as specified for individual experiments) was mixed with an equal volume containing recombinant AAV vectors (MOI 1,000-10,000) in tissue culture treated, black, glass bottom 96 well plates (Corning, Corning, NY) and incubated at room temperature (RT°) for 30 min. A total of 5×10⁴ HEK293 cells in 50 μl of media was then added to each well and the plates incubated in 5% CO₂ at 37° C. for 48 h. Cells were then lysed with 25 μl of 1× passive lysis buffer (Promega, Madison, WI) for 30 min at RT. Luciferase activity was measured on a Victor 3 multilabel plate reader (Perkin Elmer, Waltham, MA) immediately after addition of 25 μl of luciferin (Promega, Madison, WI). All read outs were normalized to controls with no antibody/antisera treatment. Recombinant AAV vectors packaging ssCBA-Luc transgenes and pre diluted in DMEM+5% FBS+P/S were utilized in this assay.

In vivo antibody neutralization assay. Each hind limb of 6-8 week old female BAlb/c mice (Jackson Laboratory, Bay Harbor, ME) was injected intramuscularly (I.M.) with 2×10¹⁰ AAV packaging CBA-Luc pre-mixed with three different monoclonal antibodies, 4E4, 5H7 and ADK1a, at 1:500, 1:50 and 1:5 dilutions, respectively, in a final volume of 20 After 4 wk post-injection, luciferase activity was measured using a Xenogen IVIS Lumina system (PerkinElmer Life Sciences/Caliper Life Sciences, Waltham, MA) at 5 min post-intraperitoneal (I.P.) injection of 175 ul of in vivo D-luciferin (120 mg/kg Nanolight, Pinetop, AZ) per mouse. Luciferase activity was measured as photons/sec/cm²/sr and analyzed using Living Image 3.2 software (Caliper Life Sciences, Waltham, MA).

Generation of anti-AAV1 mouse serum by Immunization. 1×10¹⁰ vg of wtAAV1 in 20 μl of PBS was injected intramuscularly into each hind leg of 6-8 week old, female Balb/c mice. Whole blood was collected by cardiac puncture at 4 wk post-injection and serum was isolated using standard coagulation and centrifugation protocols. Briefly, mouse blood was coagulated at RT° for 30 min and centrifuged at 2000 g for 10 min at 4° C. All serum was heat-inactivated at 55° C. for 15 min and stored at −80° C.

In vivo characterization of AAV CAM variants in mice. A dose of 1×10¹¹ vg of AAV vectors packaging the scCBh-GFP transgene cassette in 200 μl of PBS was injected into C57/B16 mice intravenously (I.V.) via the tail vein. Mice were sacrificed after 3 wk post-injection and perfused with 4% paraformaldehyde (PFA) in PBS. Multiple organs, including heart, brain, liver and kidney, were harvested. Tissues were sectioned to 50 μm thin slices by vibratome VT1200S (Leica, Welzlar, Germany) and stained for GFP with standard immunohistochemistry 3,3′-Diaminobenzidine (DAB) stain procedures described previously. At least 3 sections per organ from 3 different mice were submitted for slide scanning.. For bio-distribution analysis, 1×10¹¹ vg of AAV vectors packaging ssCBA-Luc were injected I.V. as mentioned above in Balb/C mice. After 2 wk post-injection, mice were sacrificed and perfused with 1×PBS. Multiple organs, including heart, brain, lung, liver, spleen, kidney and muscle, were harvested. DNA was harvested using DNeasy kit (Qiagen, Hilden, Germany) according to the manufacturer's instructions. Vector genome copy numbers were determined by quantitative PCR (qPCR) using as described previously using luciferase transgene primers, 5′-CCTTCGCTTCAAAAAATGGAAC-3′ (SEQ ID NO:481), and 5′-AAAAGCACTCTGATTGACAAATAC-3′ (SEQ ID NO:482). Viral genome copy numbers were normalized to mouse genomic DNA in each sample. Tissue samples were also processed for luciferase activity assays by homogenization in 1×PLB (Promega, Madison, WI) using a Qiagen TissueLyserII at a frequency of 20 hz for three 45s pulses. The homogenate was spun down, and 20 μl of supernatant mixed with 50 μl of luciferin (Promega, Madison, WI) and immediately measured using a Victor 3 multilabel plate reader (Perkin Elmer, Waltham, MA).

Intracerebroventricular (I.C.V.) injections. Postnatal day 0 (P0) C57/B16 pups which were anesthetized on ice for 2 minutes followed by stereotaxic I.C.V. injections with AAV vectors packaging the scCBh-GFP transgene cassette. A dose of 3×10⁹ vg in 3 μl of PBS was injected into the left lateral ventricle using a Hamilton 700 series syringe with a 26s gauge needle (Sigma-Aldrich, St. Louis, MO), attached to a KOPF-900 small animal stereotaxic instrument (KOPF instruments, Tujunga, CA). All neonatal injections were performed 0.5 mm relative to the sagittal sinus, 2 mm rostral to transverse sinus and 1.5 mm deep. After vector administration, mice were revived under a heat lamp and rubbed in the bedding before being placed back with the dam. Mouse brains were harvested at 2 wk post vector administrations (P14). Brains were post fixed and immunostained as described previously.

Western blots and Electron Microscopy. A total of 5×10⁹ viral genomes were re-suspended in NuPAGE LDS sample buffer (Invitrogen, Carlsbad, CA). +50 mM 1,4-Dithiothreitol (DTT). Samples were ran on NuPAGE 4-12% Bis-Tris Gel and transferred onto polyvinylidene fluoride (PVDF) membrane. (Invitrogen, Carlsbad, CA). AAV capsid proteins were detected using mouse monoclonal antibody B1 (1:50) and secondary goat anti-mouse conjugated to horseradish peroxidase (HRP) (Jackson Immuno Research Labs, West Grove, PA). For EM studies, 1×10⁹ vg/μl of virus was prepared in PBS and absorbed on a Formvar/Carbon 400 mesh, Cu grid (TED Pella, Redding, CA). Samples were negative stained with 2% uranyl acetate and analyzed using a Zeiss Supra 25 field emission scanning electron microscope.

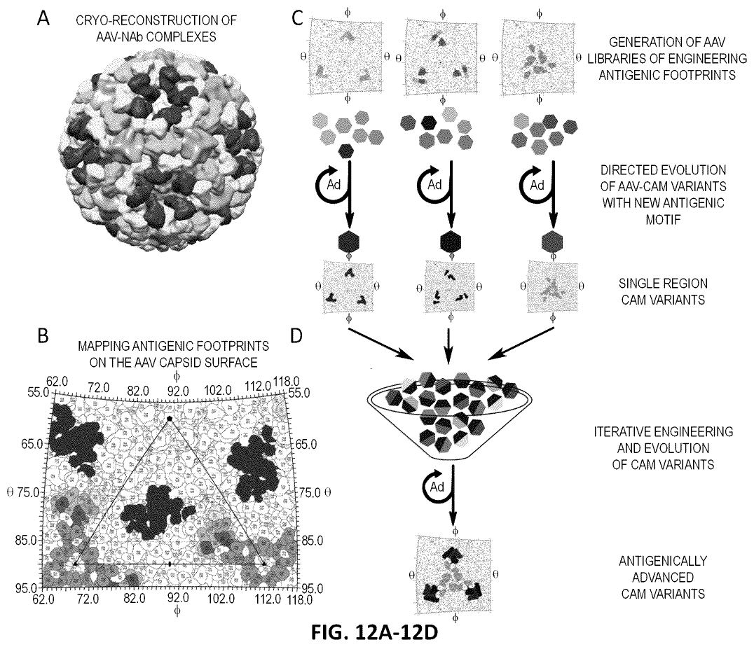

Structural analysis of AAV-Antibody complexes enables an iterative approach to evolve novel AAV variants. We analyzed previously resolved, cryo-reconstructed structures of AAV1 capsids complexed with four different fragment antigen binding (Fab) regions of anti-AAV1 monoclonal antibodies. Three-dimensional reconstruction revealed that this subset of antibodies nearly masks the entire AAV1 capsid surface (FIG. 12A). We then identified a subset of capsid surface residues (through construction of roadmap images) that lie within these antigenic footprints and are implicated in direct contact with the different antibodies (FIG. 12B). Further analysis and comparison with different AAV serotypes revealed a prominent clustering of common antigenic footprints at the 3-fold symmetry axis on the capsid surface. Specifically, amino acid residues within three surface regions, common antigenic motif 4 (CAM4; 456-AQNK-459, SEQ ID NO:483), common antigenic motif 5 (CAMS; 493-KTDNNNS-499, SEQ ID NO:484) and common antigenic motif 8 (CAM8; 588-STDPATGDVH-597, SEQ ID NO:309) were selected for saturation mutagenesis and generation of different AAV libraries. It is important to note that the different CAMs listed above are subsets of variable regions (VRs) 4, 5 and 8 outlined previously. Each AAV capsid library was then subjected to five rounds of directed evolution in vascular endothelial cells, which are highly permissive to the parental AAV1 strain (FIG. 12C). Novel AAV variants were identified and combination AAV libraries were engineered using the latter as templates. Iterative rounds of evolution and capsid engineering yielded novel antigenically advanced AAV strains characterized in the current study (FIG. 12D).

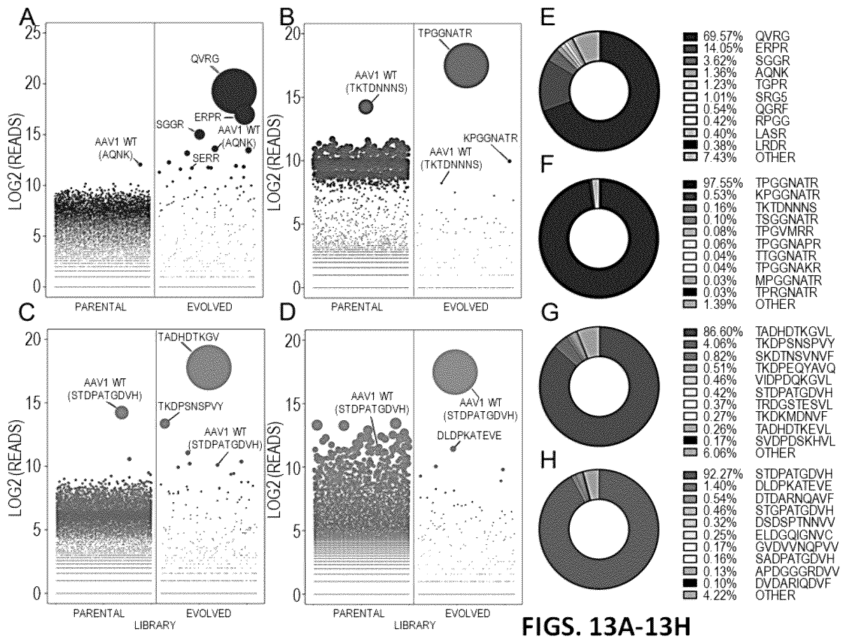

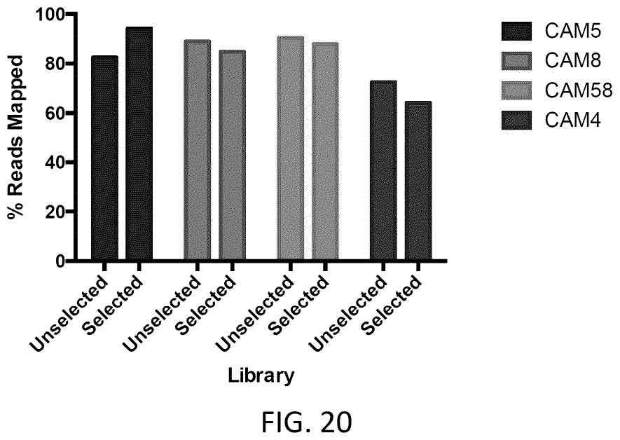

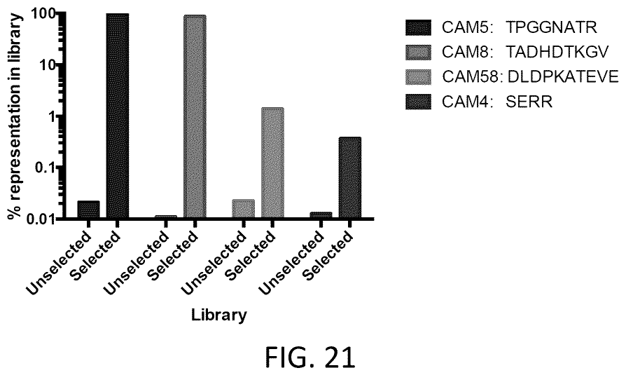

Antigenic footprints on the AAV capsid surface are remarkably plastic and evolvable. As outlined above, the AAV CAM4, CAMS, and CAM8 libraries were subjected to 5 rounds of directed evolution. Libraries were then sequenced using the MiSeq system (Illumina), wherein each unselected (parental) library was sequenced at ˜2×10⁶ reads and selected (evolved) libraries sequenced at ˜2×10⁵ reads. De-multiplexed reads were probed for mutagenized regions of interest with a custom Perl script, with a high percentage of reads mapping to these regions for all libraries (FIGS. 20-21). At both the nucleotide and amino acid level, all unselected libraries demonstrated high diversity and minimal bias towards any particular sequence, while evolved libraries showed dramatic enhancement in representation of one or more lead variants (FIGS. 13A-C, E-G). Further, within the top ten selected variants for each library, many amino acid sequences showed similarities at multiple residues (FIGS. 13E-G). For instance, in the evolved CAMS library 97.55% of sequences spanning the mutagenized region of interest read TPGGNATR (SEQ ID NO:485), while minor variants largely mimicked this sequence (FIG. 13F). In case of CAM8, we observed significant enrichment (86.6%) for a variant with amino acid residues TADHDTKGV (SEQ ID NO:486) (FIG. 13G). The evolved CAM4 library demonstrated higher plasticity (QVRG (SEQ ID NO:22), 69.57%; ERPR (SEQ ID NO:23), 14.05%; SGGR (SEQ ID NO:25), 3.62%) as evidenced by the range of amino acid residues tolerated within that antigenic region (FIG. 13A). We then generated a combination AAV library (CAM58, FIG. 13D), which carries the lead epitope from the evolved CAMS library and a randomized CAM8 region. Interestingly, subjecting this library to directed evolution yielded the wild type AAV1 sequence in the CAM8 region (92.27%), i.e., STDPATGDVH (SEQ ID NO:309) (FIG. 13H). Although a secondary variant with the sequence DLDPKATEVE (SEQ ID NO:487) was also enriched (1.4%) (FIG. 13D), the latter observation demonstrates the evolutionary and structural constraints imposed by the interaction between CAMS and CAM8 regions. These constraints were further evaluated by rational combination of different epitopes derived from these novel CAM4, 5 or 8 variants. Nevertheless, these results corroborate the notion that antigenic footprints on the AAV capsid surface are mutable and can be evolved into novel footprints, while maintaining infectivity.

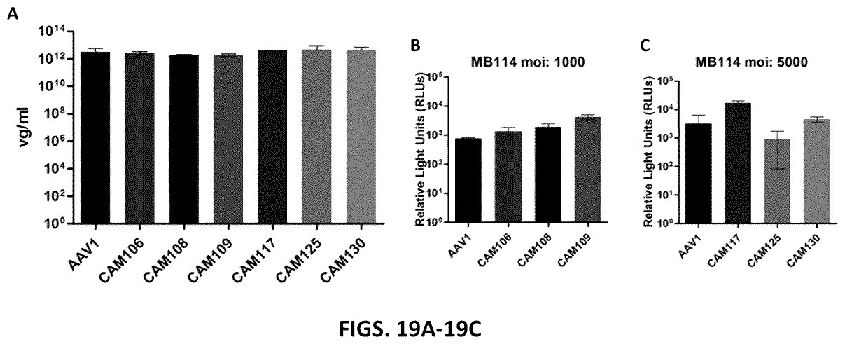

Individually evolved AAV CAM variants are similar to the parental AAV1 serotype. Multiple, evolved AAV variants were selected from each library for subsequent characterization, specifically, CAM101-107 (region 4), CAM108 (region 5) and CAM109-116 (region 8). All CAM variants packaging the ssCBA-Luc genome were produced and their transduction efficiencies assessed in vascular endothelial cells (FIGS. 19A-19C). A single CAM variant from each evolved library that displayed the highest transduction efficiency was shortlisted for further characterization. Specifically, CAM106 (456-SERR-459, SEQ ID NO:26), CAM108 (492-TPGGNATR-499, SEQ ID NO:485) and CAM109 (588-TADHDTKGVL-597, SEQ ID NO:32)) showed similar to modestly improved transduction efficiency compared to parental AAV1 on vascular endothelial cells. These observations support the notion that antigenic footprints can be re-engineered and evolved, while maintaining or improving upon the endogenous attributes of the corresponding parental AAV strain. Further evaluation of the physical properties of these lead CAM variants confirmed that yield (vector genome titers), capsid morphology (EM), and packaging efficiency (proportion of full-to-empty particles) were comparable to parental AAV1 vectors (FIGS. 19A-19C).

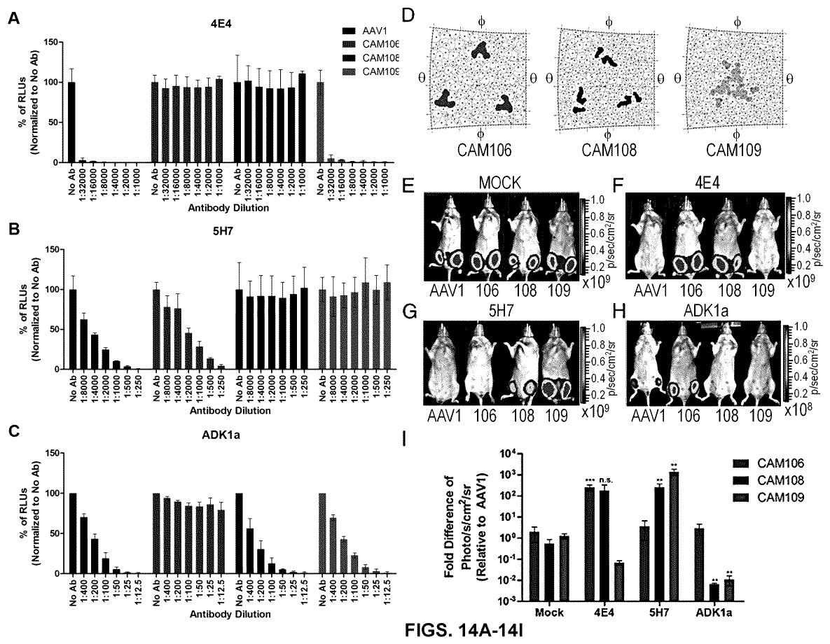

Individual CAM variants evade neutralization by monoclonal antibodies. We first evaluated the ability of single region CAM variants to escape neutralization by mouse monoclonal antibodies, ADK1a, 4E4 and 5H7 described previously. As shown in FIGS. 14A-C, each CAM variant shows a distinct NAb escape profile. As expected, parental AAV1 was neutralized by all MAbs tested at different dilutions. The CAM106 and CAM108 variants were resistant to neutralization by 4E4, while CAM109 was completely neutralized similar to AAV1 (FIG. 14A). Next, we determined that CAM108 and CAM109 both escape neutralization by 5H7, whereas CAM106 was significantly affected by 5H7 similar to AAV1 (FIG. 14B). With ADK1a, CAM106 was completely resistant to neutralization, while CAM108 and CAM109 were both effectively neutralized (FIG. 14C).

In vivo neutralization profile of CAM variants against monoclonal antibodies. To further test whether the ability of CAM variants to escape neutralization can be reproduced in vivo, AAV1 and CAM variants packaging ssCBA-Luc were mixed with the corresponding MAbs and injected intramuscularly into mice. In the absence of MAbs, all CAM variants and AAV1 showed similar luciferase transgene expression in mouse muscle (FIG. 14E). In the presence of antibodies, the neutralization profiles of the CAM variants corroborated results from in vitro studies. Briefly, CAM106 was resistant to ADK1a and 4E4, while CAM108 efficiently transduces mouse muscle in the presence of 4E4 or 5H7 and CAM109 evades 5H7 with high efficiency. Importantly, AAV1 transduction of mouse muscle was completely abolished when co-administered with any of these antibodies (FIGS. 14F-H). Quantitative analysis of luciferase transgene expression by CAM variants normalized to AAV1 confirmed these observations (FIG. 14I).

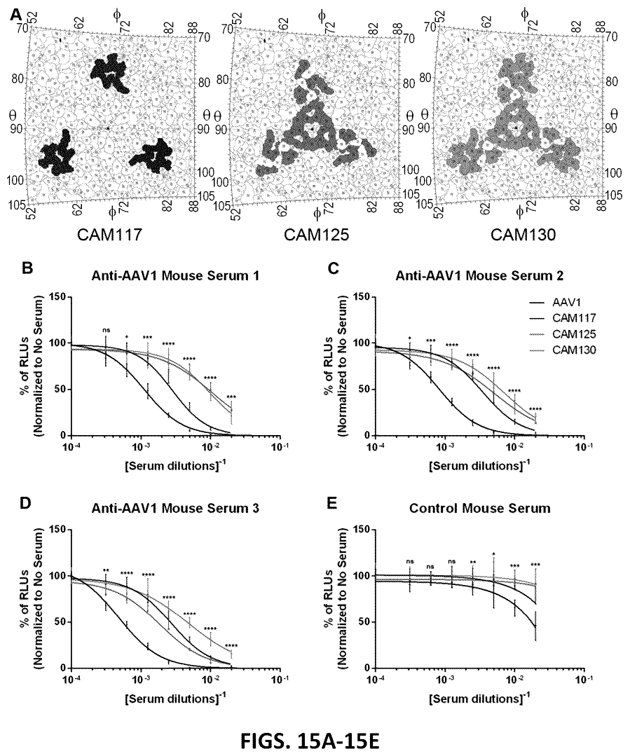

Iterative engineering of complex antigenic footprints on single region CAM variants. Based on promising results from MAb neutralization studies, we hypothesized that combining different, evolved antigenic footprints will allow better NAb evasion. To achieve such, we generated four variants through a combination of rational mutagenesis, library generation and iterative evolution. First, we observed that rational combination of antigenic footprints from CAM106 and CAM108 yielded a functional and stable AAV variant, dubbed CAM117 (FIG. 15A). However, we observed that amino acid residues constituting antigenically advanced footprints on CAM108 and CAM109 were not structurally compatible (reduced viral titer) In order to facilitate structural compatibility, we generated a new AAV capsid library using CAM108 as a template and by carrying out saturation mutagenesis of amino acid residues in region 8. After 3 iterative cycles of directed evolution on vascular endothelial cells, several viable variants were generated (FIG. 15A). After initial characterization, CAM125 (region 5, 492-TPGGNATR-499 (SEQ ID NO:485); region 8, 588-DLDPKATEVE-597 (SEQ ID NO:487)) was selected for further analysis. We then iteratively engineered a third variant (CAM130) by grafting the evolved antigenic footprint from CAM106 onto CAM125. The CAM130 variant contains the following amino acid residues in three distinct antigenic footprints—region 4, 456-SERR-459 (SEQ ID NO:26; region 5, 492-TPGGNATR-499 (SEQ ID NO:485) and region 8, 588-DLDPKATEVE-597 (SEQ ID NO:487) (FIG. 15A). All three iteratively engineered variants, CAM117, CAM125 and CAM130 show similar physical attributes compared to parental AAV1 with regard to titer and proportion of full-to-empty particles (FIGS. 19A-19C).

CAM117, CAM 125 and CAM130 escape neutralizing antisera from pre-immunized mice. To test whether antigenically advanced CAM variants can demonstrate escape from polyclonal neutralizing antibodies found in serum, we sero-converted mice by immunization with wild type AAV1 capsids. Overall, while antisera obtained from individual mice efficiently neutralized AAV1, CAM117, CAM125 and CAM130 display increased resistance to neutralization (FIGS. 15B-D). Briefly, we tested antisera dilutions ranging over two orders of magnitude (1:3200 to 1:50) to generate sigmoidal neutralization curves. As seen in FIGS. 15B-D, when compared to AAV1, the CAM variants show a dramatic shift to the right indicating improved ability to evade anti-AAV1 serum. In particular, the serum concentration required for 50% neutralization of transduction (ND₅₀) is significantly higher in case of each CAM variant compared to parental AAV1 in each individual subject (FIGS. 15B-D). Furthermore, we observed an incremental ability to evade NAbs with each iterative engineering/evolution step. Specifically, the most antigenically advanced variant, CAM130 displays a 8-16 fold improvement in ND₅₀ values (FIGS. 15B-D). These results corroborate the notion that antigenic footprints on AAV capsid are modular and cumulative in their ability to mediate NAb evasion. A similar, but less robust trend was observed with regard to the neutralizing potential of serum obtained from naïve mice as control (FIG. 15E).

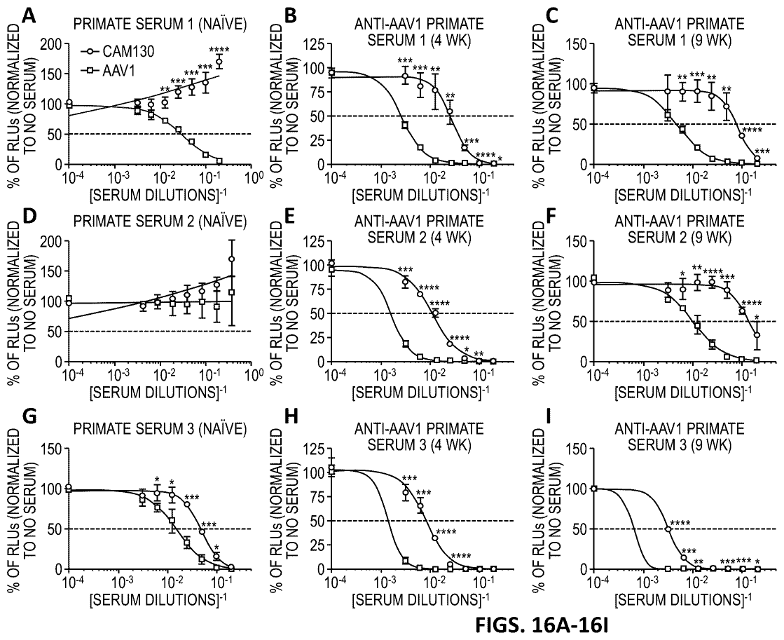

CAM130 efficiently evades neutralization by non-human primate antisera. To validate whether our approach can be translated in larger animal models, we tested the ability of AAV1 and the lead variant, CAM130 to evade NAbs generated in non-human primates. Briefly, we subjected AAV vectors to neutralization assays using serum collected at three different time points—pre-immunization (naïve), 4 wks and 9 wks post-immunization. All macaques sero-converted after immunization with NAb titers at the highest levels in week 4 and declining at week 9 in subjects 1 and 2, and increased potency at week 9 in subject 3 (FIGS. 16A-I). Moreover, naïve sera from subjects 1 and 3 prior to immunization were able to neutralize AAV1 effectively (FIGS. 16A and 16G). We tested antisera dilutions ranging over two orders of magnitude (1:320 to 1:5) to generate neutralization curves as described earlier. Antisera obtained at 4 wks after immunization neutralized AAV1 effectively at ND₅₀>1:320. In contrast, CAM130 displayed a significant shift to the right and improved resistance to neutralization compared to AAV1 by 4-16 fold (FIGS. 16B, 16E, 16H). A similar trend and enhancement in resistance to NAbs was observed in the case of CAM130 when evaluating antisera obtained at 9 wks post-immunization (FIGS. 16C, 16F, 16I). Further, these results strongly support the notion that antigenicity of AAV capsids can be re-engineered to escape broadly neutralizing antibodies from different animal species on the basis of structural cues obtained from mouse MAb footprints.

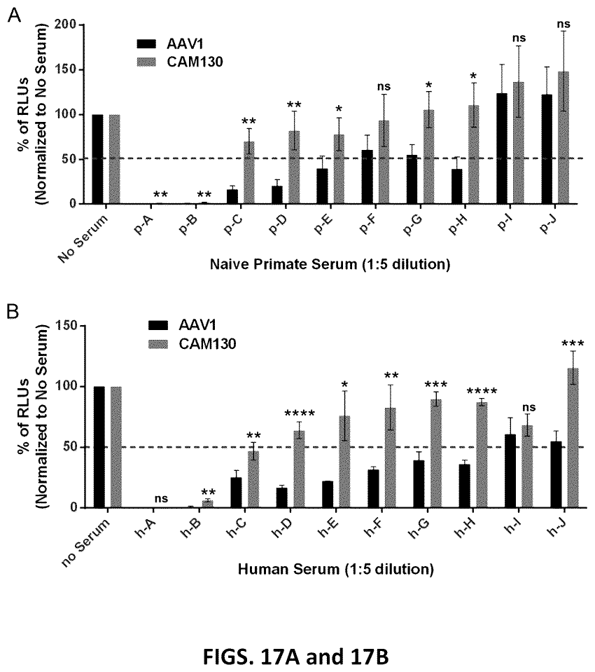

CAM130 efficiently evades NAbs in primate and human sera. To test whether CAM130 can evade NAbs in the general non-human primate and human population, we tested serum samples obtained from a cohort of 10 subjects each. We evaluated a fixed serum dilution of 1:5 to reflect currently mandated exclusion criteria employed in ongoing clinical trials for hemophilia and other indications requiring systemic AAV administration. As seen in FIG. 17A, primate subjects p-A and p-B displayed high NAb titers that completely neutralized both AAV1 and CAM130. At the other end of the spectrum, subjects p-I and p-J showed no pre-existing immunity to AAV capsids and did not effectively neutralize AAV1 or CAM130. However, serum samples for subjects p-C through p-H efficiently neutralized AAV1 and reduced transduction efficiency below 50% of untreated controls. In contrast, serum samples p-C through p-H were unable to neutralize the antigenically advanced CAM130 variant. Thus, CAM130 shows exceptional NAb evasion in this cohort by evading 8 out of 10 serum samples (FIG. 17A). We then utilized a similar approach to test serum from 10 human subjects. Using clinically relevant exclusion criteria (1:5 dilution), we segregated the human sera into two high titer (h-A and h-B), six intermediate titer (h-C through h-H) and two modest titer sub-groups that neutralized AAV1 effectively. Strikingly, CAM130 was able to evade polyclonal NAbs in human sera for 8/10 samples tested (FIG. 17B). Taken together, these studies strongly support the notion that the antigenically advanced CAM130 variant can significantly expand the patient cohort.

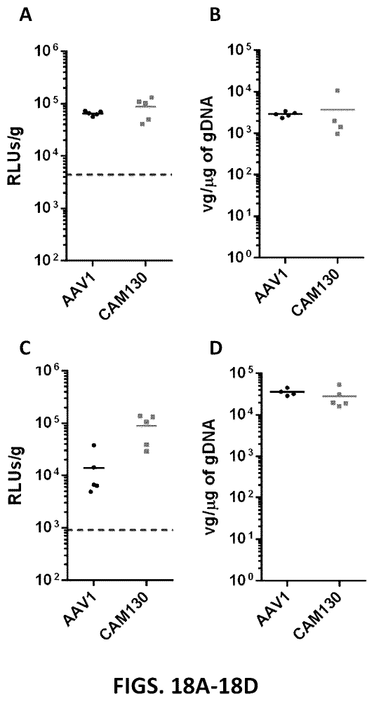

CAM130 displays a favorable transduction profile in vivo. We compared the in vivo tissue tropism, transduction efficiency and biodistribution of CAM130 to the parental AAV1 strain in mice. A dose of 1×10¹¹ vg/mouse of AAV vectors packaging scCBh-GFP was injected intravenously into 6-8 week old female BALB/c mice via the tail vein. At 2 wks post injection, CAM130 showed an enhanced cardiac GFP expression profile compared to AAV1, while differences in the liver were unremarkable. In particular, more GFP-positive cardiac myofibers are detectable in CAM130 treated animals compared to the AAV1 cohort. We then administered 1×10¹¹ vg/mouse of AAV vectors packaging ssCBA-Luc genomes intravenously as described above. In contrast to GFP expression from self-complementary CAM130 vectors, no significant differences were noted in luciferase activity within the heart for ssCAM130 vs. AAV1-treated mice (FIG. 18A). However, a modest, albeit statistically insignificant increase in luciferase expression was observed within the liver (FIG. 18C). Transduction efficiencies in other major organs, i.e., lung, brain, kidney and spleen, were low. Importantly, no differences were noted in the systemic biodistribution of CAM130 and AAV1 vectors. Consistent with earlier reports, ˜10-fold higher vector genome copy numbers were detected in the liver compared to cardiac tissue for both CAM130 and AAV1 vectors (FIGS. 18B and 18D).

To further compare the potency and tropism of CAM130 to AAV1, we evaluated the transduction profiles of the latter two strains following CNS administration. A dose of 3×10⁹ vg/mouse of AAV1 or CAM130 packaging scCBh-GFP genomes was injected by intra-CSF administration in neonatal mice. Both AAV1 and CAM130 spread well within the brain with a general preference for transducing the ipsilateral side more readily than the contralateral hemisphere. Similar to cardiac tissue, a greater number of GFP-positive cells are observed in the case of CAM130 compared to AAV1. In particular, CAM130 appears to transduce a greater number of neurons, particularly within the motor cortex, cortex and most prominently in the hippocampus. The potential mechanism(s) for the improved transduction profile displayed by CAM130 in cardiac and CNS tissue could potentially arise from post-entry trafficking events that are currently under investigation. More importantly, these in vivo results confirm that antigenic footprints on AAV capsids can be engineered to effectively evade NAbs, while simultaneously controlling cellular/tissue tropism as well as biodistribution profile and improving potency.

Similarly, AAV1e mutants demonstrate robust and neuron-specific gene expression in the brain following intracranial administration. Different AAV1e vectors packaging an scGFP expression cassette were administered into the cerebrospinal fluid of P0 mice by stereotaxic injection into lateral ventricles. The vector dose administered was 3×10⁹ vector genomes/animal. Mice were sacrificed at 3 weeks post-injection and brains processed using DAB immunohistochemistry and image reporter gene expression in cerebellum, olfactory bulb, cortex and hippocampus.

### Example 4: AAV8e Antibody Evading Mutants

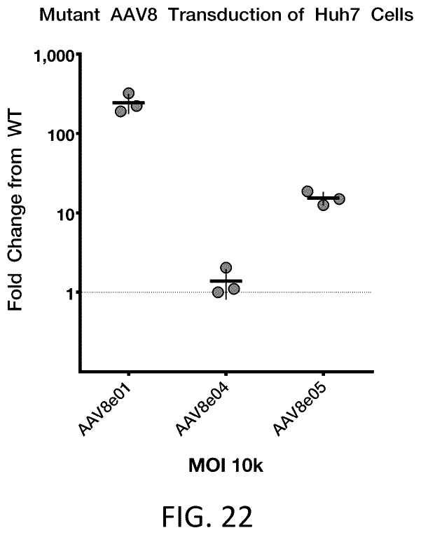

Evolved mutants AAV8e01, AAV8e04 and AAV8e05 demonstrate improved transduction in comparison with parental AAV8 isolate in human hepatocarcinoma cells (Huh7). Briefly, cells were incubated with different AAV8 derived variants at 10,000 vector genomes per cell for 24 hours. Quantitation of luciferase transgene activity revealed over 2 log increase in transduction efficiency of AAV8e01 over AAV8 (dotted line); over 1 log order increase for AAV8e05 and ˜2-fold increase in the case of AAV8e04. These results corroborate the generation of novel AAV8 variants that demonstrate robust transduction of transformed human hepatocytes in culture compared to the state-of-the-art natural isolate. These results are shown in FIG. 22.

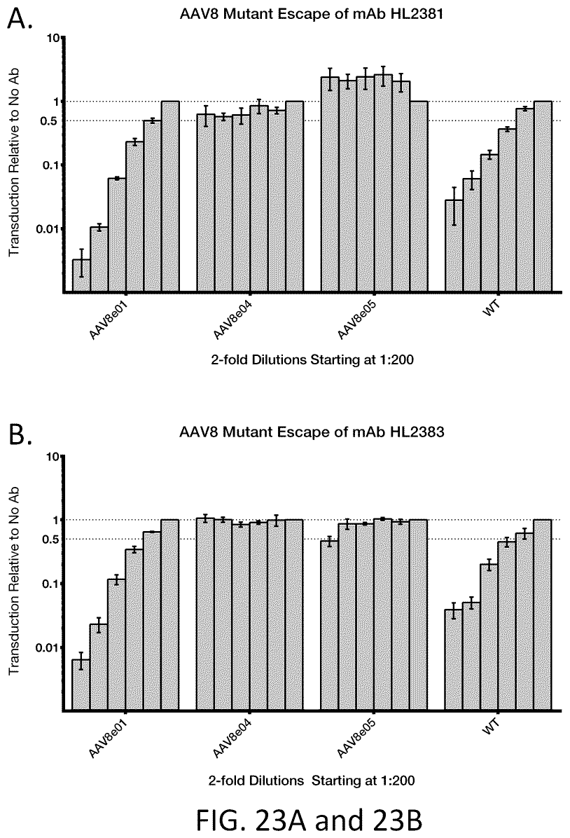

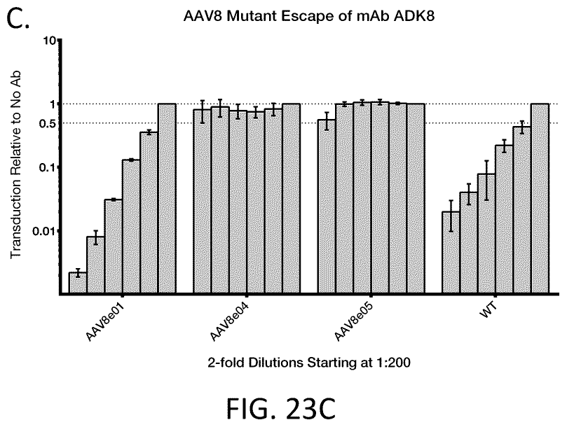

AAV8e mutants demonstrate the ability to escape neutralization by mouse monoclonal antibodies generated specifically against AAV8. Briefly, human hepatocarcinoma cells were incubated with different AAV8e mutants or wild type (WT) AAV8 vectors packaging luciferase transgene cassettes with or without neutralizing antibodies. Each monoclonal antibody (mAb) was directed against different antigenic epitopes located on the AAV8 capsid surface. As shown in FIGS. 23A-23C, AAV8e04 and AAV8e05 escape neutralization by mAbs HL2381 (FIG. 23A), HL2383 (FIG. 23B) and ADK8 (FIG. 23C) tested at different dilutions. In contrast, the parental AAV8 strain is neutralized effectively under these conditions.

Nonlimiting examples of AAV8e mutants of this invention are listed in Table 9.

TABLE 9
AAV8e mutants

| Name | Clone | Sequence | Description |
|---|---|---|---|
| AAV8e01 | CAM84a | 455-SNGRGV-460 | Single 8CAM-4a |
| | | (SEQ ID NO: 488) | |
| AAV8e02 | CAM84b | 455-VNTSLVG-461 | Single 8CAM-4b |
| | | (SEQ ID NO: 489) | |
| AAV8e03 | CAM84c | 455-IRGAGAV-461 | Single 8CAM-4c |
| | | (SEQ ID NO: 490) | |
| AAV8e04 | CAM85a | 494-YPGGNYK-501 | Single 8CAM-5a |
| | | (SEQ ID NO: 491) | |
| AAV8e05 | CAM88a | 586-KQKNVN-591 | Single 8CAM-8a |
| | | (SEQ ID NO: 492) | |
| AAV8e06 | CAM88b | 586-RMSSIK-591 | Single 8CAM-8b |
| | | (SEQ ID NO: 493) | |
| AAV8e07 | CAM845a | 455-SNGRGV-460 | Double |
| | | (SEQ ID NO: 488) + | 8CAM-4a-5a |
| | | 494-YPGGNYK-501 | |
| | | (SEQ ID NO: 491) | |
| AAV8e08 | CAM848a | 455-SNGRGV-460 | Double |
| | | (SEQ ID NO: 488) + | 8CAM-4a-8a |
| | | 586-KQKNVN-591 | |
| | | (SEQ ID NO: 492) | |

The foregoing is illustrative of the present invention, and is not to be construed as limiting thereof. The invention is defined by the following claims, with equivalents of the claims to be included therein.

SEQUENCES
AAV1 capsid protein
(GenBank Accession No. AAD27757)
(SEQ ID NO: 1)

| | |
|---|---|
| 1 | MAADGYLPDW LEDNLSEGIR EWWDLKPGAP KPKANQQKQD |
| | DGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPLG LVEEGAKTAP GKKRPVEQSP QEPDSSSGIG |
| | KTGQQPAKKR LNFGQTGDSE |
| 181 | SVPDPQPLGE PPATPAAVGP TTMASGGGAP MADNNEGADG |
| | VGNASGNWHC DSTWLGDRVI |
| 241 | TTSTRTWALP TYNNHLYKQI SSASTGASND NHYFGYSTPW |
| | GYFDFNRFHC HFSPRDWQRL |
| 301 | INNNWGFRPK RLNFKLFNIQ VKEVTTNDGV TTIANNLTST |
| | VQVFSDSEYQ LPYVLGSAHQ |
| 361 | GCLPPFPADV FMIPQYGYLT LNNGSQAVGR SSFYCLEYFP |
| | SQMLRTGNNF TFSYTFEEVP |
| 421 | FHSSYAHSQS LDRLMNPLID QYLYYLNRTQ NQSGSAQNKD |
| | LLFSRGSPAG MSVQPKNWLP |
| 481 | GPCYRQQRVS KTKTDNNNSN FTWTGASKYN LNGRESIINP |
| | GTAMASHKDD EDKFFPMSGV |
| 541 | MIFGKESAGA SNTALDNVMI TDEEEIKATN PVATERFGTV |
| | AVNFQSSSTD PATGDVHAMG |
| 601 | ALPGMVWQDR DVYLQGPIWA KIPHTDGHFH PSPLMGGFGL |
| | KNPPPQILIK NTPVPANPPA |
| 661 | EFSATKFASF ITQYSTGQVS VEIEWELQKE NSKRWNPEVQ |
| | YTSNYAKSAN VDFTVDNNGL |
| 721 | YTEPRPIGTR YLTRPL |

AAV2 capsid protein
(GenBank Accession No. YP 680426)
(SEQ ID NO: 2)

| | |
|---|---|
| 1 | MAADGYLPDW LEDTLSEGIR QWWKLKPGPP PPKPAERHKD |
| | DSRGLVLPGY KYLGPFNGLD |
| 61 | KGEPVNEADA AALEHDKAYD RQLDSGDNPY LKYNHADAEF |
| | QERLKEDTSF GGNLGRAVFQ |
| 121 | AKKRVLEPLG LVEEPVKTAP GKKRPVEHSP VEPDSSSGTG |
| | KAGQQPARKR LNFGQTGDAD |
| 181 | SVPDPQPLGQ PPAAPSGLGT NTMATGSGAP MADNNEGADG |
| | VGNSSGNWHC DSTWMGDRVI |
| 241 | TTSTRTWALP TYNNHLYKQI SSQSGASNDN HYFGYSTPWG |
| | YFDFNRFHCH FSPRDWQRLI |
| 301 | NNNWGFRPKR LNFKLFNIQV KEVTQNDGTT TIANNLTSTV |
| | QVFTDSEYQL PYVLGSAHQG |
| 361 | CLPPFPADVF MVPQYGYLTL NNGSQAVGRS SFYCLEYFPS |
| | QMLRTGNNFT FSYTFEDVPF |
| 421 | HSSYAHSQSL DRLMNPLIDQ YLYYLSRTNT PSGTTTQSRL |
| | QFSQAGASDI RDQSRNWLPG |
| 481 | PCYRQQRVSK TSADNNNSEY SWTGATKYHL NGRDSLVNPG |
| | PAMASHKDDE EKFFPQSGVL |
| 541 | IFGKQGSEKT NVDIEKVMIT DEEEIRTTNP VATEQYGSVS |
| | TNLQRGNRQA ATADVNTQGV |
| 601 | LPGMVWQDRD VYLQGPIWAK IPHTDGHFHP SPLMGGFGLK |
| | HPPPQILIKN TPVPANPSTT |
| 661 | FSAAKFASFI TQYSTGQVSV EIEWELQKEN SKRWNPEIQY |
| | TSNYNKSVNV DFTVDTNGVY |
| 721 | SEPRPIGTRY LTRNL |

AAV3 capsid protein
(GenBank Accession No. AAC55049)
(SEQ ID NO: 3)

| | |
|---|---|
| 1 | MAADGYLPDW LEDNLSEGIR EWWALKPGVP QPKANQQHQD |
| | NRRGLVLPGY KYLGPGNGLD |
| 61 | KGEPVNEADA AALEHDKAYD QQLKAGDNPY LKYNHADAEF |
| | QERLQEDTSF GGNLGRAVFQ |
| 121 | AKKRILEPLG LVEEAAKTAP GKKGAVDQSP QEPDSSSGVG |
| | KSGKQPARKR LNFGQTGDSE |
| 181 | SVPDPQPLGE PPAAPTSLGS NTMASGGGAP MADNNEGADG |
| | VGNSSGNWHC DSQWLGDRVI |
| 241 | TTSTRTWALP TYNNHLYKQI SSQSGASNDN HYFGYSTPWG |
| | YFDFNRFHCH FSPRDWQRLI |
| 301 | NNNWGFRPKK LSFKLFNIQV RGVTQNDGTT TIANNLTSTV |
| | QVFTDSEYQL PYVLGSAHQG |
| 361 | CLPPFPADVF MVPQYGYLTL NNGSQAVGRS SFYCLEYFPS |
| | QMLRTGNNFQ FSYTFEDVPF |
| 421 | HSSYAHSQSL DRLMNPLIDQ YLYYLNRTQG TTSGTTNQSR |
| | LLFSQAGPQS MSLQARNWLP |
| 481 | GPCYRQQRLS KTANDNNNSN FPWTAASKYH LNGRDSLVNP |
| | GPAMASHKDD EEKFFPMHGN |
| 541 | LIFGKEGTTA SNAELDNVMI TDEEEIRTTN PVATEQYGTV |
| | ANNLQSSNTA PTTGTVNHQG |
| 601 | ALPGMVWQDR DVYLQGPIWA KIPHTDGHFH PSPLMGGFGL |
| | KHPPPQIMIK NTPVPANPPT |
| 661 | TFSPAKFASF ITQYSTGQVS VEIEWELQKE NSKRWNPEIQ |
| | YTSNYNKSVN VDFTVDTNGV |
| 721 | YSEPRPIGTR YLTRNL |

AAV4 capsid protein
(GenBank Accession No. NP 044927)
(SEQ ID NO: 4)

| | |
|---|---|
| 1 | MTDGYLPDWL EDNLSEGVRE WWALQPGAPK PKANQQHQDN |
| | ARGLVLPGYK YLGPGNGLDK |
| 61 | GEPVNAADAA ALEHDKAYDQ QLKAGDNPYL KYNHADAEFQ |
| | QRLQGDTSFG GNLGRAVFQA |
| 121 | KKRVLEPLGL VEQAGETAPG KKRPLIESPQ QPDSSTGIGK |
| | KGKQPAKKKL VFEDETGAGD |
| 181 | GPPEGSTSGA MSDDSEMRAA AGGAAVEGGQ GADGVGNASG |
| | DWHCDSTWSE GHVTTTSTRT |
| 241 | WVLPTYNNHL YKRLGESLQS NTYNGFSTPW GYFDFNRFHC |
| | HFSPRDWQRL INNNWGMRPK |
| 301 | AMRVKIFNIQ VKEVTTSNGE TTVANNLTST VQIFADSSYE |
| | LPYVMDAGQE GSLPPFPNDV |
| 361 | FMVPQYGYCG LVTGNTSQQQ TDRNAFYCLE YFPSQMLRTG |
| | NNFEITYSFE KVPFHSMYAH |
| 421 | SQSLDRLMNP LIDQYLWGLQ STTTGTTLNA GTATTNFTKL |
| | RPTNFSNFKK NWLPGPSIKQ |
| 481 | QGFSKTANQN YKIPATGSDS LIKYETHSTL DGRWSALTPG |
| | PPMATAGPAD SKFSNSQLIF |
| 541 | AGPKQNGNTA TVPGTLIFTS EEELAATNAT DTDMWGNLPG |
| | GDQSNSNLPT VDRLTALGAV |
| 601 | PGMVWQNRDI YYQGPIWAKI PHTDGHFHPS PLIGGFGLKH |
| | PPPQIFIKNT PVPANPATTF |
| 661 | SSTPVNSFIT QYSTGQVSVQ IDWEIQKERS KRWNPEVQFT |
| | SNYGQQNSLL WAPDAAGKYT |
| 721 | EPRAIGTRYL THHL |

AAV5 capsid protein
(GenBank Accession No. AAD13756)
(SEQ ID NO: 5)

| | |
|---|---|
| 1 | MSFVDHPPDW LEEVGEGLRE FLGLEAGPPK PKPNQQHQDQ |
| | ARGLVLPGYN YLGPGNGLDR |
| 61 | GEPVNRADEV AREHDISYNE QLEAGDNPYL KYNHADAEFQ |
| | EKLADDTSFG GNLGKAVFQA |
| 121 | KKRVLEPFGL VEEGAKTAPT GKRIDDHFPK RKKARTEEDS |
| | KPSTSSDAEA GPSGSQQLQI |
| 181 | PAQPASSLGA DTMSAGGGGP LGDNNQGADG VGNASGDWHC |
| | DSTWMGDRVV TKSTRTWVLP |
| 241 | SYNNHQYREI KSGSVDGSNA NAYFGYSTPW GYFDFNRFHS |
| | HWSPRDWQRL INNYWGFRPR |
| 301 | SLRVKIFNIQ VKEVTVQDST TTIANNLTST VQVFTDDDYQ |
| | LPYVVGNGTE GCLPAFPPQV |
| 361 | FTLPQYGYAT LNRDNTENPT ERSSFFCLEY FPSKMLRTGN |
| | NFEFTYNFEE VPFHSSFAPS |
| 421 | QNLFKLANPL VDQYLYRFVS TNNTGGVQFN KNLAGRYANT |
| | YKNWFPGPMG RTQGWNLGSG |
| 481 | VNRASVSAFA TTNRMELEGA SYQVPPQPNG MTNNLQGSNT |
| | YALENTMIFN SQPANPGTTA |
| 541 | TYLEGNMLIT SESETQPVNR VAYNVGGQMA TNNQSSTTAP |
| | ATGTYNLQEI VPGSVWMERD |
| 601 | VYLQGPIWAK IPETGAHFHP SPAMGGFGLK HPPPMMLIKN |
| | TPVPGNITSF SDVPVSSFIT |
| 661 | QYSTGQVTVE MEWELKKENS KRWNPEIQYT NNYNDPQFVD |
| | FAPDSTGEYR TTRPIGTRYL |
| 721 | TRPL |

AAV6 capsid protein
(GenBank Accession No. AAB95450)
(SEQ ID NO: 6)

| | |
|---|---|
| 1 | MAADGYLPDW LEDNLSEGIR EWWDLKPGAP KPKANQQKQD |
| | DGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPFG LVEEGAKTAP GKKRPVEQSP QEPDSSSGIG |
| | KTGQQPAKKR LNFGQTGDSE |
| 181 | SVPDPQPLGE PPATPAAVGP TTMASGGGAP MADNNEGADG |
| | VGNASGNWHC DSTWLGDRVI |
| 241 | TTSTRTWALP TYNNHLYKQI SSASTGASND NHYFGYSTPW |
| | GYFDFNRFHC HFSPRDWQRL |
| 301 | INNNWGFRPK RLNFKLFNIQ VKEVTTNDGV TTIANNLTST |
| | VQVFSDSEYQ LPYVLGSAHQ |
| 361 | GCLPPFPADV FMIPQYGYLT LNNGSQAVGR SSFYCLEYFP |
| | SQMLRTGNNF TFSYTFEDVP |
| 421 | FHSSYAHSQS LDRLMNPLID QYLYYLNRTQ NQSGSAQNKD |
| | LLFSRGSPAG MSVQPKNWLP |
| 481 | GPCYRQQRVS KTKTDNNNSN FTWTGASKYN LNGRESIINP |
| | GTAMASHKDD KDKFFPMSGV |
| 541 | MIFGKESAGA SNTALDNVMI TDEEEIKATN PVATERFGTV |
| | AVNLQSSSTD PATGDVHVMG |
| 601 | ALPGMVWQDR DVYLQGPIWA KIPHTDGHFH PSPLMGGFGL |
| | KHPPPQILIK NTPVPANPPA |
| 661 | EFSATKFASF ITQYSTGQVS VEIEWELQKE NSKRWNPEVQ |
| | YTSNYAKSAN VDFTVDNNGL |
| 721 | YTEPRPIGTR YLTRPL |

AAV7 capsid protein
(GenBank Accession No. AAN03855)
(SEQ ID NO: 7)

| | |
|---|---|
| 1 | MAADGYLPDW LEDNLSEGIR EWWDLKPGAP KPKANQQKQD |
| | NGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPLG LVEEGAKTAP AKKRPVEPSP QRSPDSSTGI |
| | GKKGQQPARK RLNFGQTGDS |
| 181 | ESVPDPQPLG EPPAAPSSVG SGTVAAGGGA PMADNNEGAD |
| | GVGNASGNWH CDSTWLGDRV |
| 241 | ITTSTRTWAL PTYNNHLYKQ ISSETAGSTN DNTYFGYSTP |
| | WGYFDFNRFH CHFSPRDWQR |
| 301 | LINNNWGFRP KKLRFKLFNI QVKEVTTNDG VTTIANNLTS |
| | TIQVFSDSEY QLPYVLGSAH |
| 361 | QGCLPPFPAD VFMIPQYGYL TLNNGSQSVG RSSFYCLEYF |
| | PSQMLRTGNN FEFSYSFEDV |
| 421 | PFHSSYAHSQ SLDRLMNPLI DQYLYYLART QSNPGGTAGN |
| | RELQFYQGGP STMAEQAKNW |
| 481 | LPGPCFRQQR VSKTLDQNNN SNFAWTGATK YHLNGRNSLV |
| | NPGVAMATHK DDEDRFFPSS |
| 541 | GVLIFGKTGA TNKTTLENVL MTNEEEIRPT NPVATEEYGI |
| | VSSNLQAANT AAQTQVVNNQ |
| 601 | GALPGMVWQN RDVYLQGPIW AKIPHTDGNF HPSPLMGGFG |
| | LKHPPPQILI KNTPVPANPP |
| 661 | EVFTPAKFAS FITQYSTGQV SVEIEWELQK ENSKRWNPEI |
| | QYTSNFEKQT GVDFAVDSQG |
| 721 | VYSEPRPIGT RYLTRNL |

AAV8 capsid protein
(GenBank Accession No. AAN03857)
(SEQ ID NO: 8)

| | |
|---|---|
| 1 | MAADGYLPDW LEDNLSEGIR EWWALKPGAP KPKANQQKQD |
| | DGRGLVLPGY KYLGPFNGLD |
| 61 | KGEPVNAADA AALEHDKAYD QQLQAGDNPY LRYNHADAEF |
| | QERLQEDTSF GGNLGRAVFQ |
| 121 | AKKRVLEPLG LVEEGAKTAP GKKRPVEPSP QRSPDSSTGI |
| | GKKGQQPARK RLNFGQTGDS |
| 181 | ESVPDPQPLG EPPAAPSGVG PNTMAAGGGA PMADNNEGAD |
| | GVGSSSGNWH CDSTWLGDRV |
| 241 | ITTSTRTWAL PTYNNHLYKQ ISNGTSGGAT NDNTYFGYST |
| | PWGYFDFNRF HCHFSPRDWQ |
| 301 | RLINNNWGFR PKRLSFKLFN IQVKEVTQNE GTKTIANNLT |
| | STIQVFTDSE YQLPYVLGSA |
| 361 | HQGCLPPFPA DVFMIPQYGY LTLNNGSQAV GRSSFYCLEY |
| | FPSQMLRTGN NFQFTYTFED |
| 421 | VPFHSSYAHS QSLDRLMNPL IDQYLYYLSR TQTTGGTANT |
| | QTLGFSQGGP NTMANQAKNW |
| 481 | LPGPCYRQQR VSTTTGQNNN SNFAWTAGTK YHLNGRNSLA |
| | NPGIAMATHK DDEERFFPSN |
| 541 | GILIFGKQNA ARDNADYSDV MLTSEEEIKT TNPVATEEYG |
| | IVADNLQQQN TAPQIGTVNS |
| 601 | QGALPGMVWQ NRDVYLQGPI WAKIPHTDGN FHPSPLMGGF |
| | GLKHPPPQIL IKNTPVPADP |
| 661 | PTTFNQSKLN SFITQYSTGQ VSVEIEWELQ KENSKRWNPE |
| | IQYTSNYYKS TSVDFAVNTE |
| 721 | GVYSEPRPIG TRYLTRNL |

AAV9 capsid protein
(GenBank Accession No. AAS99264)
(SEQ ID NO: 9)

| | |
|---|---|
| 1 | MAADGYLPDW LEDNLSEGIR EWWALKPGAP QPKANQQHQD |
| | NARGLVLPGY KYLGPGNGLD |
| 61 | KGEPVNAADA AALEHDKAYD QQLKAGDNPY LKYNHADAEF |
| | QERLKEDTSF GGNLGRAVFQ |
| 121 | AKKRLLEPLG LVEEAAKTAP GKKRPVEQSP QEPDSSAGIG |
| | KSGAQPAKKR LNFGQTGDTE |
| 181 | SVPDPQPIGE PPAAPSGVGS LTMASGGGAP VADNNEGADG |
| | VGSSSGNWHC DSQWLGDRVI |
| 241 | TTSTRTWALP TYNNHLYKQI SNSTSGGSSN DNAYFGYSTP |
| | WGYFDFNRFH CHFSPRDWQR |
| 301 | LINNNWGFRP KRLNFKLFNI QVKEVTDNNG VKTIANNLTS |
| | TVQVFTDSDY QLPYVLGSAH |
| 361 | EGCLPPFPAD VFMIPQYGYL TLNDGSQAVG RSSFYCLEYF |
| | PSQMLRTGNN FQFSYEFENV |
| 421 | PFHSSYAHSQ SLDRLMNPLI DQYLYYLSKT INGSGQNQQT |
| | LKFSVAGPSN MAVQGRNYIP |
| 481 | GPSYRQQRVS TTVTQNNNSE FAWPGASSWA LNGRNSLMNP |
| | GPAMASHKEG EDRFFPLSGS |
| 541 | LIFGKQGTGR DNVDADKVMI TNEEEIKTTN PVATESYGQV |
| | ATNHQSAQAQ AQTGWVQNQG |
| 601 | ILPGMVWQDR DVYLQGPIWA KIPHTDGNFH PSPLMGGFGM |
| | KHPPPQILIK NTPVPADPPT |
| 661 | AFNKDKLNSF ITQYSTGQVS VEIEWELQKE NSKRWNPEIQ |
| | YTSNYYKSNN VEFAVNTEGV |
| 721 | YSEPRPIGTR YLTRNL |

AAVrh.8 capsid protein
(GenBank Accession No. AA088183)
(SEQ ID NO: 10)

| | |
|---|---|
| 1 | MAADGYLPDW LEDNLSEGIR EWWDLKPGAP KPKANQQKQD |
| | DGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPLG LVEEGAKTAP GKKRPVEQSP QEPDSSSGIG |
| | KTGQQPAKKR LNFGQTGDSE |
| 181 | SVPDPQPLGE PPAAPSGLGP NTMASGGGAP MADNNEGADG |
| | VGNSSGNWHC DSTWLGDRVI |
| 241 | TTSTRTWALP TYNNHLYKQI SNGTSGGSTN DNTYFGYSTP |
| | WGYFDFNRFH CHFSPRDWQR |
| 301 | LINNNWGFRP KRLNFKLFNI QVKEVTTNEG TKTIANNLTS |
| | TVQVFTDSEY QLPYVLGSAH |
| 361 | QGCLPPFPAD VFMVPQYGYL TLNNGSQALG RSSFYCLEYF |
| | PSQMLRTGNN FQFSYTFEDV |
| 421 | PFHSSYAHSQ SLDRLMNPLI DQYLYYLVRT QTTGTGGTQT |
| | LAFSQAGPSS MANQARNWVP |
| 481 | GPCYRQQRVS TTTNQNNNSN FAWTGAAKFK LNGRDSLMNP |
| | GVAMASHKDD DDRFFPSSGV |
| 541 | LIFGKQGAGN DGVDYSQVLI TDEEEIKATN PVATEEYGAV |
| | AINNQAANTQ AQTGLVHNQG |
| 601 | VIPGMVWQNR DVYLQGPIWA KIPHTDGNFH PSPLMGGFGL |
| | KHPPPQILIK NTPVPADPPL |
| 661 | TFNQAKLNSF ITQYSTGQVS VEIEWELQKE NSKRWNPEIQ |
| | YTSNYYKSTN VDFAVNTEGV |
| 721 | YSEPRPIGTR YLTRNL |

AAVrh.10 capsid protein
(GenBank Accession No. AA088201)
(SEQ ID NO: 11)

| | |
|---|---|
| 1 | MAADGYLPDW LEDNLSEGIR EWWDLKPGAP KPKANQQKQD |
| | DGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPLG LVEEGAKTAP GKKRPVEPSP QRSPDSSTGI |
| | GKKGQQPAKK RLNFGQTGDS |
| 181 | ESVPDPQPIG EPPAGPSGLG SGTMAAGGGA PMADNNEGAD |
| | GVGSSSGNWH CDSTWLGDRV |
| 241 | ITTSTRTWAL PTYNNHLYKQ ISNGTSGGST NDNTYFGYST |
| | PWGYFDFNRF HCHFSPRDWQ |
| 301 | RLINNNWGFR PKRLNFKLFN IQVKEVTQNE GTKTIANNLT |
| | STIQVFTDSE YQLPYVLGSA |
| 361 | HQGCLPPFPA DVFMIPQYGY LTLNNGSQAV GRSSFYCLEY |
| | FPSQMLRTGN NFEFSYQFED |
| 421 | VPFHSSYAHS QSLDRLMNPL IDQYLYYLSR TQSTGGTAGT |
| | QQLLFSQAGP NNMSAQAKNW |
| 481 | LPGPCYRQQR VSTTLSQNNN SNFAWTGATK YHLNGRDSLV |
| | NPGVAMATHK DDEERFFPSS |
| 541 | GVLMFGKQGA GKDNVDYSSV MLTSEEEIKT TNPVATEQYG |
| | VVADNLQQQN AAPIVGAVNS |
| 601 | QGALPGMVWQ NRDVYLQGPI WAKIPHTDGN FHPSPLMGGF |
| | GLKHPPPQIL IKNTPVPADP |
| 661 | PTTFSQAKLA SFITQYSTGQ VSVEIEWELQ KENSKRWNPE |
| | IQYTSNYYKS TNVDFAVNTD |
| 721 | GTYSEPRPIG TRYLTRNL |

AAV10 capsid protein
(GenBank Accession No. AAT46337)
(SEQ ID NO: 12)

| | |
|---|---|
| 1 | MAADGYLPDW LEDNLSEGIR EWWDLKPGAP KPKANQQKQD |
| | DGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPLG LVEEAAKTAP GKKRPVEPSP QRSPDSSTGI |
| | GKKGQQPAKK RLNFGQTGES |
| 181 | ESVPDPQPIG EPPAGPSGLG SGTMAAGGGA PMADNNEGAD |
| | GVGSSSGNWH CDSTWLGDRV |
| 241 | ITTSTRTWAL PTYNNHLYKQ ISNGTSGGST NDNTYFGYST |
| | PWGYFDFNRF HCHFSPRDWQ |
| 301 | RLINNNWGFR PKRLSFKLFN IQVKEVTQNE GTKTIANNLT |
| | STIQVFTDSE YQLPYVLGSA |
| 361 | HQGCLPPFPA DVFMIPQYGY LTLNNGSQAV GRSSFYCLEY |
| | FPSQMLRTGN NFEFSYTFED |
| 421 | VPFHSSYAHS QSLDRLMNPL IDQYLYYLSR TQSTGGTQGT |
| | QQLLFSQAGP ANMSAQAKNW |
| 481 | LPGPCYRQQR VSTTLSQNNN SNFAWTGATK YHLNGRDSLV |
| | NPGVAMATHK DDEERFFPSS |
| 541 | GVLMFGKQGA GRDNVDYSSV MLTSEEEIKT TNPVATEQYG |
| | VVADNLQQAN TGPIVGNVNS |
| 601 | QGALPGMVWQ NRDVYLQGPI WAKIPHTDGN FHPSPLMGGF |
| | GLKHPPPQIL IKNTPVPADP |
| 661 | PTTFSQAKLA SFITQYSTGQ VSVEIEWELQ KENSKRWNPE |
| | IQYTSNYYKS TNVDFAVNTE |
| 721 | GTYSEPRPIG TRYLTRNL |

AAV11 capsid protein
(GenBank Accession No. AAT46339)
(SEQ ID NO: 13)

| | |
|---|---|
| 1 | MAADGYLPDW LEDNLSEGIR EWWDLKPGAP KPKANQQKQD |
| | DGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPLG LVEEGAKTAP GKKRPLESPQ EPDSSSGIGK |
| | KGKQPARKRL NFEEDTGAGD |
| 181 | GPPEGSDTSA MSSDIEMRAA PGGNAVDAGQ GSDGVGNASG |
| | DWHCDSTWSE GKVTTTSTRT |
| 241 | WVLPTYNNHL YLRLGTTSSS NTYNGFSTPW GYFDFNRFHC |
| | HFSPRDWQRL INNNWGLRPK |
| 301 | AMRVKIFNIQ VKEVTTSNGE TTVANNLTST VQIFADSSYE |
| | LPYVMDAGQE GSLPPFPNDV |
| 361 | FMVPQYGYCG IVTGENQNQT DRNAFYCLEY FPSQMLRTGN |
| | NFEMAYNFEK VPFHSMYAHS |
| 421 | QSLDRLMNPL LDQYLWHLQS TTSGETLNQG NAATTFGKIR |
| | SGDFAFYRKN WLPGPCVKQQ |
| 481 | RFSKTASQNY KIPASGGNAL LKYDTHYTLN NRWSNIAPGP |
| | PMATAGPSDG DFSNAQLIFP |
| 541 | GPSVTGNTTT SANNLLFTSE EEIAATNPRD TDMFGQIADN |
| | NQNATTAPIT GNVTAMGVLP |
| 601 | GMVWQNRDIY YQGPIWAKIP HADGHFHPSP LIGGFGLKHP |
| | PPQIFIKNTP VPANPATTFT |
| 661 | AARVDSFITQ YSTGQVAVQI EWEIEKERSK RWNPEVQFTS |
| | NYGNQSSMLW APDTTGKYTE |
| 721 | PRVIGSRYLT NHL |

AAV12 capsid protein
(GenBank Accession No. ABI16639)
(SEQ ID NO: 14)

| | |
|---|---|
| 1 | MAADGYLPDW LEDNLSEGIR EWWALKPGAP QPKANQQHQD |
| | NGRGLVLPGY KYLGPFNGLD |
| 61 | KGEPVNEADA AALEHDKAYD KQLEQGDNPY LKYNHADAEF |
| | QQRLATDTSF GGNLGRAVFQ |
| 121 | AKKRILEPLG LVEEGVKTAP GKKRPLEKTP NRPTNPDSGK |
| | APAKKKQKDG EPADSARRTL |
| 181 | DFEDSGAGDG PPEGSSSGEM SHDAEMRAAP GGNAVEAGQG |
| | ADGVGNASGD WHCDSTWSEG |
| 241 | RVTTTSTRTW VLPTYNNHLY LRIGTTANSN TYNGFSTPWG |
| | YFDFNRFHCH FSPRDWQRLI |
| 301 | NNNWGLRPKS MRVKIFNIQV KEVTTSNGET TVANNLTSTV |
| | QIFADSTYEL PYVMDAGQEG |
| 361 | SFPPFPNDVF MVPQYGYCGV VTGKNQNQTD RNAFYCLEYF |
| | PSQMLRTGNN FEVSYQFEKV |
| 421 | PFHSMYAHSQ SLDRMMNPLL DQYLWHLQST TTGNSLNQGT |
| | ATTTYGKITT GDFAYYRKNW |
| 481 | LPGACIKQQK FSKNANQNYK IPASGGDALL KYDTHTTLNG |
| | RWSNMAPGPP MATAGAGDSD |
| 541 | FSNSQLIFAG PNPSGNTTTS SNNLLFTSEE EIATTNPRDT |
| | DMFGQIADNN QNATTAPHIA |
| 601 | NLDAMGIVPG MVWQNRDIYY QGPIWAKVPH TDGHFHPSPL |
| | MGGFGLKHPP PQIFIKNTPV |
| 661 | PANPNTTFSA ARINSFLTQY STGQVAVQID WEIQKEHSKR |
| | WNPEVQFTSN YGTQNSMLWA |
| 721 | PDNAGNYHEL RAIGSRFLTH HL |

AAVrh.32.33 capsid protein
(GenBank Accession No. ACB55318)
(SEQ ID NO: 15)

| | |
|---|---|
| 1 | MAADGYLPDW LEDNLSEGIR EWWDLKPGAP KPKANQQKQD |
| | DGRGLVLPGY KYLGPFNGLD |
| 61 | KGEPVNAADA AALEHDKAYD QQLKAGDNPY LRYNHADAEF |
| | QERLQEDTSF GGNLGRAVFQ |
| 121 | AKKRVLEPLG LVEEGAKTAP GKKRPLESPQ EPDSSSGIGK |
| | KGKQPAKKRL NFEEDTGAGD |
| 181 | GPPEGSDTSA MSSDIEMRAA PGGNAVDAGQ GSDGVGNASG |
| | DWHCDSTWSE GKVTTTSTRT |
| 241 | WVLPTYNNHL YLRLGTTSNS NTYNGFSTPW GYFDFNRFHC |
| | HFSPRDWQRL INNNWGLRPK |
| 301 | AMRVKIFNIQ VKEVTTSNGE TTVANNLTST VQIFADSSYE |
| | LPYVMDAGQE GSLPPFPNDV |
| 361 | FMVPQYGYCG IVTGENQNQT DRNAFYCLEY FPSQMLRTGN |
| | NFEMAYNFEK VPFHSMYAHS |
| 421 | QSLDRLMNPL LDQYLWHLQS TTSGETLNQG NAATTFGKIR |
| | SGDFAFYRKN WLPGPCVKQQ |
| 481 | RFSKTASQNY KIPASGGNAL LKYDTHYTLN NRWSNIAPGP |
| | PMATAGPSDG DFSNAQLIFP |
| 541 | GPSVTGNTTT SANNLLFTSE EEIAATNPRD TDMFGQIADN |
| | NQNATTAPIT GNVTAMGVLP |
| 601 | GMVWQNRDIY YQGPIWAKIP HADGHFHPSP LIGGFGLKHP |
| | PPQIFIKNTP VPANPATTFT |
| 661 | AARVDSFITQ YSTGQVAVQI EWEIEKERSK RWNPEVQFTS |
| | NYGNQSSMLW APDTTGKYTE |
| 721 | PRVIGSRYLT NHL |

Bovine AAV capsid protein
(GenBank Accession No. YP 024971)
(SEQ ID NO: 16)

| | |
|---|---|
| 1 | MSFVDHPPDW LESIGDGFRE FLGLEAGPPK PKANQQKQDN |
| | ARGLVLPGYK YLGPGNGLDK |
| 61 | GDPVNFADEV AREHDLSYQK QLEAGDNPYL KYNHADAEFQ |
| | EKLASDTSFG GNLGKAVFQA |
| 121 | KKRILEPLGL VETPDKTAPA AKKRPLEQSP QEPDSSSGVG |
| | KKGKQPARKR LNFDDEPGAG |
| 181 | DGPPPEGPSS GAMSTETEMR AAAGGNGGDA GQGAEGVGNA |
| | SGDWHCDSTW SESHVTTTST |
| 241 | RTWVLPTYNN HLYLRLGSSN ASDTFNGFST PWGYFDFNRF |
| | HCHFSPRDWQ RLINNHWGLR |
| 301 | PKSMQVRIFN IQVKEVTTSN GETTVSNNLT STVQIFADST |
| | YELPYVMDAG QEGSLPPFPN |
| 361 | DVFMVPQYGY CGLVTGGSSQ NQTDRNAFYC LEYFPSQMLR |
| | TGNNFEMVYK FENVPFHSMY |
| 421 | AHSQSLDRLM NPLLDQYLWE LQSTTSGGTL NQGNSATNFA |
| | KLTKTNFSGY RKNWLPGPMM |
| 481 | KQQRFSKTAS QNYKIPQGRN NSLLHYETRT TLDGRWSNFA |
| | PGTAMATAAN DATDFSQAQL |
| 541 | IFAGPNITGN TTTDANNLMF TSEDELRATN PRDTDLFGHL |
| | ATNQQNATTV PTVDDVDGVG |
| 601 | VYPGMVWQDR DIYYQGPIWA KIPHTDGHFH PSPLIGGFGL |
| | KSPPPQIFIK NTPVPANPAT |
| 661 | TFSPARINSF ITQYSTGQVA VKIEWEIQKE RSKRWNPEVQ |
| | FTSNYGAQDS LLWAPDNAGA |
| 721 | YKEPRAIGSR YLTNHL |

Avian AAV ATCC VR-865 capsid protein
(GenBank Accession No. NP 852781)
(SEQ ID NO: 17)

| | |
|---|---|
| 1 | MSLISDAIPD WLERLVKKGV NAAADFYHLE SGPPRPKANQ |
| | QTQESLEKDD SRGLVFPGYN |
| 61 | YLGPFNGLDK GEPVNEADAA ALEHDKAYDL EIKDGHNPYF |
| | EYNEADRRFQ ERLKDDTSFG |
| 121 | GNLGKAIFQA KKRVLEPFGL VEDSKTAPTG DKRKGEDEPR |
| | LPDTSSQTPK KNKKPRKERP |
| 181 | SGGAEDPGEG TSSNAGAAAP ASSVGSSIMA EGGGGPVGDA |
| | GQGADGVGNS SGNWHCDSQW |
| 241 | LENGVVTRTT RTWVLPSYNN HLYKRIQGPS GGDNNNKFFG |
| | FSTPWGYFDY NRFHCHFSPR |
| 301 | DWQRLINNNW GIRPKAMRFR LFNIQVKEVT VQDFNTTIGN |
| | NLTSTVQVFA DKDYQLPYVL |
| 361 | GSATEGTFPP FPADIYTIPQ YGYCTLNYNN EAVDRSAFYC |
| | LDYFPSDMLR TGNNFEFTYT |
| 421 | FEDVPFHSMF AHNQTLDRLM NPLVDQYLWA FSSVSQAGSS |
| | GRALHYSRAT KTNMAAQYRN |
| 481 | WLPGPFFRDQ QIFTGASNIT KNNVFSVWEK GKQWELDNRT |
| | NLMQPGPAAA TTFSGEPDRQ |
| 541 | AMQNTLAFSR TVYDQTTATT DRNQILITNE DEIRPTNSVG |
| | IDAWGAVPTN NQSIVTPGTR |
| 601 | AAVNNQGALP GMVWQNRDIY PTGTHLAKIP DTDNHFHPSP |
| | LIGRFGCKHP PPQIFIKNTP |
| 661 | VPANPSETFQ TAKVASFINQ YSTGQCTVEI FWELKKETSK |
| | RWNPEIQFTS NFGNAADIQF |
| 721 | AVSDTGSYSE PRPIGTRYLT KPL |

1. An adeno associated virus (AAV) capsid protein, wherein the capsid protein comprises one or more of the following substitutions, wherein the amino acids are numbered according to the amino acid sequence of SEQ ID NO:8:
(a) a substitution of the amino acids corresponding to amino acids 455-460 with the sequence SNGRGV (SEQ ID NO:488), a substitution of the amino acids corresponding to amino acids 455-461 with the sequence VNTSING (SEQ ID NO:489), or a substitution of the amino acids corresponding to amino acids 455-461 with the sequence IRGAGAV (SEQ ID NO:490),
(b) a substitution of the amino acids corresponding to amino acids 494-501 with the sequence YPGGNYK (SEQ ID NO:491), and/or
(c) a substitution of the amino acids corresponding to amino acids 586-591 with the sequence KQKNVN (SEQ ID NO:492) or RMSSIK (SEQ ID NO:493)
wherein the capsid protein comprising one or more of the substitutions (a)-(c) has an amino acid sequence that is at least 90% identical to the amino acid sequence of SEQ ID NO:8.

2. (canceled)

3. An adeno associated virus (AAV) capsid protein, wherein the capsid protein comprises a substitution of the amino acids corresponding to amino acids 455-460 with the sequence SNGRGV (SEQ ID NO:488), wherein the amino acids are numbered according to the amino acid sequence of SEQ ID NO:8, and wherein the capsid protein has an amino acid sequence that is at least 90% identical to the amino acid sequence of SEQ ID NO:8.

4. The AAV capsid protein of claim 3, wherein the capsid protein has an amino acid sequence that is at least 95% identical to the amino acid sequence of SEQ ID NO:8.

5. The AAV capsid protein of claim 3, wherein the capsid protein has an amino acid sequence that is at least 98% identical to the amino acid sequence of SEQ ID NO:8.

6. An adeno associated virus (AAV) capsid protein, wherein the capsid protein comprises one or more of the following substitutions, wherein the amino acids are numbered according to the amino acid sequence of SEQ ID NO:1:
(a) a substitution of amino acids corresponding to amino acids 456 to 459 with the amino acid sequence SERR (SEQ ID NO:26);
(b) a substitution of amino acids corresponding to amino acids 492 to 499 with the amino acid sequence TPGGNATR (SEQ ID NO:485); and
(c) a substitution of amino acids corresponding to amino acids 588 to 597 with the amino acid sequence DLDPKATEVE (SEQ ID NO:487),
wherein the capsid protein comprising one or more of the substitutions (a)-(c) has an amino acid sequence that is at least 90% identical to the amino acid sequence of SEQ ID NO:1.

7. The AAV capsid protein of claim 6, wherein the capsid protein comprises all three of substitutions (a)-(c).

8. The AAV capsid protein of claim 7, wherein the capsid protein has an amino acid sequence that is at least 97% identical to the amino acid sequence of SEQ ID NO:8.

9. A nucleic acid comprising a nucleic acid sequence encoding the capsid protein of claim 1.

10. A nucleic acid comprising a nucleic acid sequence encoding the capsid protein of claim 3.

11. A nucleic acid comprising a nucleic acid sequence encoding the capsid protein of claim 6.

12. A nucleic acid comprising a nucleic acid sequence encoding the capsid protein of claim 7.

13. A nucleic acid comprising a nucleic acid sequence encoding the capsid protein of claim 8.

14. An isolated cell comprising the nucleic acid of claim 9.

15. An isolated cell comprising the nucleic acid of claim 10.

16. An isolated cell comprising the nucleic acid of claim 12.

17. An isolated cell comprising the nucleic acid of claim 13.

18. A method of producing an adeno-associated virus (AAV) capsid protein, the method comprising:
a. culturing the isolated cell of claim 14; and
b. collecting the AAV capsid protein from the cell.

19. A method of producing an adeno-associated virus (AAV) capsid protein, the method comprising:
a. culturing the isolated cell of claim 15; and
b. collecting the AAV capsid protein from the cell.

20. A method of producing an adeno-associated virus (AAV) capsid protein, the method comprising:
a. culturing the isolated cell of claim 16; and
b. collecting the AAV capsid protein from the cell.


SEQUENCE LISTING (527 sequences)

SEQ ID NO: 1 - protein, 736 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, adeno-associated virus [sequence omitted]

SEQ ID NO: 3 - protein, 736 aa, adeno-associated virus
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, adeno-associated virus
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 11 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 12 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 13 - protein, 733 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 14 - protein, 742 aa, adeno-associated virus
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 15 - protein, 733 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 16 - protein, 736 aa, adeno-associated virus
MSFVDHPPDWLESIGDGFREFLGLEAGPPKPKANQQKQDNARGLVLPGYKYLGPGNGLDKGDPVNFADEVAREHDLSYQKQLEAGDNPYLKYNHADAEFQEKLASDTSFGGNLGKAVFQAKKRILEPLGLVETPDKTAPAAKKRPLEQSPQEPDSSSGVGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 17 - protein, 743 aa, adeno-associated virus
MSLISDAIPDWLERLVKKGVNAAADFYHLESGPPRPKANQQTQESLEKDDSRGLVFPGYNYLGPFNGLDKGEPVNEADAAALEHDKAYDLEIKDGHNPYFEYNEADRRFQERLKDDTSFGGNLGKAIFQAKKRVLEPFGLVEDSKTAPTGDKRKGEDEPRLPDTSSQTPKKNKKPRKERPSGGAEDPGEGTSSNAGAAAPASSVGSSIMAEGGGGPVGDAGQGADGVGNSSGNWHCDSQWLENGVVTRTTRTWVLPSYNNHLYKRIQGPSGGDNNNKFFGFSTPWGYFDYNRFHCHFSPRDWQRLINNNWGIRPKAMRFRLFNIQVKEVTVQDFNTTIGNNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQAGSSGRALHYSRATKTNMAAQYRNWLPGPFFRDQQIFTGASNITKNNVFSVWEKGKQWELDNRTNLMQPGPAAATTFSGEPDRQAMQNTLAFSRTVYDQTTATTDRNQILITNEDEIRPTNSVGIDAWGAVPTNNQSIVTPGTRAAVNNQGALPGMVWQNRDIYPTGTHLAKIPDTDNHFHPSPLIGRFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 18-21 - skipped (4 entries)

SEQ ID NO: 22 - protein, 4 aa, synthetic construct
  REGION 1..4: note = AAV capsid protein common antigenic motif (CAM) substitution sequence
QVRG

SEQ ID NO: 23 - protein, 4 aa, synthetic construct
  REGION 1..4: note = AAV capsid protein common antigenic motif (CAM) substitution sequence
ERPR

SEQ ID NO: 24 - protein, 4 aa, synthetic construct
  REGION 1..4: note = AAV capsid protein common antigenic motif (CAM) substitution sequence
GRGG

SEQ ID NO: 25 - protein, 4 aa, synthetic construct
  REGION 1..4: note = AAV capsid protein common antigenic motif (CAM) substitution sequence
SGGR

SEQ ID NO: 26 - protein, 4 aa, synthetic construct
  REGION 1..4: note = AAV capsid protein common antigenic motif (CAM) substitution sequence
SERR

SEQ ID NO: 27 - protein, 4 aa, synthetic construct
  REGION 1..4: note = AAV capsid protein common antigenic motif (CAM) substitution sequence
LRGG

SEQ ID NO: 28-29 - skipped (2 entries)

SEQ ID NO: 30 - protein, 7 aa, synthetic construct
  REGION 1..7: note = AAV capsid protein common antigenic motif (CAM) substitution sequence
PGGNATR

SEQ ID NO: 31 - skipped

SEQ ID NO: 32 - protein, 10 aa, synthetic construct
  REGION 1..10: note = AAV capsid protein common antigenic motif (CAM) substitution sequence
TADHDTKGVL

SEQ ID NO: 33 - protein, 10 aa, synthetic construct
  REGION 1..10: note = AAV capsid protein common antigenic motif (CAM) substitution sequence
VVDPDKKGVL

SEQ ID NO: 34 - protein, 10 aa, synthetic construct
  REGION 1..10: note = AAV capsid protein common antigenic motif (CAM) substitution sequence
AKDTGPLNVM

SEQ ID NO: 35 - protein, 10 aa, synthetic construct
  REGION 1..10: note = AAV capsid protein common antigenic motif (CAM) substitution sequence
QTDAKDNGVQ

SEQ ID NO: 36 - protein, 10 aa, synthetic construct
  REGION 1..10: note = AAV capsid protein common antigenic motif (CAM) substitution sequence
DKDPWLNDVI

SEQ ID NO: 37 - protein, 10 aa, synthetic construct
  REGION 1..10: note = AAV capsid protein common antigenic motif (CAM) substitution sequence
TRDGSTESVL

SEQ ID NO: 38 - protein, 10 aa, synthetic construct
  REGION 1..10: note = AAV capsid protein common antigenic motif (CAM) substitution sequence
VIDPDQKGVL

SEQ ID NO: 39 - protein, 10 aa, synthetic construct
  REGION 1..10: note = AAV capsid protein common antigenic motif (CAM) substitution sequence
VNDMSNYMVH

SEQ ID NO: 40-161 - skipped (122 entries)

SEQ ID NO: 162 - protein, 5 aa, synthetic construct
  REGION 1..5: note = Targeting peptide sequence
FVFLP

SEQ ID NO: 163 - skipped

SEQ ID NO: 164 - protein, 4 aa, synthetic construct
  REGION 1..4: note = Targeting peptide sequence
RGNR

SEQ ID NO: 165 - protein, 7 aa, synthetic construct
  REGION 1..7: note = Targeting peptide sequence
  SITE 7: note = MISC_FEATURE - Xaa can be G or S
NSVRDLX

SEQ ID NO: 166 - protein, 7 aa, synthetic construct
  REGION 1..7: note = Targeting peptide sequence
PRSVTVP

SEQ ID NO: 167 - protein, 7 aa, synthetic construct
  REGION 1..7: note = Targeting peptide sequence
  SITE 6: note = misc_feature - Xaa can be any naturally occurring amino acid
  SITE 7: note = MISC_FEATURE - Xaa can be S or A
NSVSSXX

SEQ ID NO: 168 - protein, 6 aa, synthetic construct
  REGION 1..6: note = Targeting peptide sequence
NGRAHA

SEQ ID NO: 169 - protein, 7 aa, synthetic construct
  REGION 1..7: note = Targeting peptide sequence
QPEHSST

SEQ ID NO: 170 - protein, 7 aa, synthetic construct
  REGION 1..7: note = Targeting peptide sequence
VNTANST

SEQ ID NO: 171 - protein, 7 aa, synthetic construct
  REGION 1..7: note = Targeting peptide sequence
HGPMQKS

SEQ ID NO: 172 - protein, 7 aa, synthetic construct
  REGION 1..7: note = Targeting peptide sequence
PHKPPLA

SEQ ID NO: 173 - protein, 7 aa, synthetic construct
  REGION 1..7: note = Targeting peptide sequence
IKNNEMW

SEQ ID NO: 174 - protein, 7 aa, synthetic construct
  REGION 1..7: note = Targeting peptide sequence
RNLDTPM

SEQ ID NO: 175 - protein, 7 aa, synthetic construct
  REGION 1..7: note = Targeting peptide sequence
VDSHRQS

SEQ ID NO: 176 - protein, 7 aa, synthetic construct
  REGION 1..7: note = Targeting peptide sequence
YDSKTKT

SEQ ID NO: 177 - protein, 7 aa, synthetic construct
  REGION 1..7: note = Targeting peptide sequence
SQLPHQK

SEQ ID NO: 178 - protein, 7 aa, synthetic construct
  REGION 1..7: note = Targeting peptide sequence
STMQQNT

SEQ ID NO: 179 - protein, 7 aa, synthetic construct
  REGION 1..7: note = Targeting peptide sequence
TERYMTQ

SEQ ID NO: 180 - protein, 7 aa, synthetic construct
  REGION 1..7: note = Targeting peptide sequence
QPEHSST

SEQ ID NO: 181 - protein, 7 aa, synthetic construct
  REGION 1..7: note = Targeting peptide sequence
DASLSTS

SEQ ID NO: 182 - protein, 7 aa, synthetic construct
  REGION 1..7: note = Targeting peptide sequence
DLPNKKT

SEQ ID NO: 183 - protein, 7 aa, synthetic construct
  REGION 1..7: note = Targeting peptide sequence
DLTAARL

SEQ ID NO: 184 - protein, 7 aa, synthetic construct
  REGION 1..7: note = Targeting peptide sequence
EPHQFNY

SEQ ID NO: 185 - protein, 7 aa, synthetic construct
  REGION 1..7: note = Targeting peptide sequence
EPQSNHT

SEQ ID NO: 186 - protein, 7 aa, synthetic construct
  REGION 1..7: note = Targeting peptide sequence
MSSWPSQ

SEQ ID NO: 187 - protein, 7 aa, synthetic construct
  REGION 1..7: note = Targeting peptide sequence
NPKHNAT

SEQ ID NO: 188 - protein, 7 aa, synthetic construct
  REGION 1..7: note = Targeting peptide sequence
PDGMRTT

SEQ ID NO: 189 - protein, 7 aa, synthetic construct
  REGION 1..7: note = Targeting peptide sequence
PNNNKTT

SEQ ID NO: 190 - protein, 7 aa, synthetic construct
  REGION 1..7: note = Targeting peptide sequence
QSTTHDS

SEQ ID NO: 191 - protein, 7 aa, synthetic construct
  REGION 1..7: note = Targeting peptide sequence
TGSKQKQ

SEQ ID NO: 192 - protein, 7 aa, synthetic construct
  REGION 1..7: note = Targeting peptide sequence
SLKHQAL

SEQ ID NO: 193 - protein, 7 aa, synthetic construct
  REGION 1..7: note = Targeting peptide sequence
SPIDGEQ

SEQ ID NO: 194 - protein, 8 aa, synthetic construct
  REGION 1..8: note = Targeting peptide sequence
WIFPWIQL

SEQ ID NO: 195 - protein, 9 aa, synthetic construct
  REGION 1..9: note = Targeting peptide sequence
CDCRGDCFC

SEQ ID NO: 196 - protein, 5 aa, synthetic construct
  REGION 1..5: note = Targeting peptide sequence
CNGRC

SEQ ID NO: 197 - protein, 7 aa, synthetic construct
  REGION 1..7: note = Targeting peptide sequence
CPRECES

SEQ ID NO: 198 - protein, 10 aa, synthetic construct
  REGION 1..10: note = Targeting peptide sequence
CTTHWGFTLC

SEQ ID NO: 199 - protein, 9 aa, synthetic construct
  REGION 1..9: note = Targeting peptide sequence
CGRRAGGSC

SEQ ID NO: 200 - protein, 9 aa, synthetic construct
  REGION 1..9: note = Targeting peptide sequence
CKGGRAKDC

SEQ ID NO: 201 - protein, 9 aa, synthetic construct
  REGION 1..9: note = Targeting peptide sequence
CVPELGHEC

SEQ ID NO: 202 - protein, 9 aa, synthetic construct
  REGION 1..9: note = Targeting peptide sequence
CRRETAWAK

SEQ ID NO: 203 - protein, 14 aa, synthetic construct
  REGION 1..14: note = Targeting peptide sequence
VSWFSHRYSPFAVS

SEQ ID NO: 204 - protein, 13 aa, synthetic construct
  REGION 1..13: note = Targeting peptide sequence
GYRDGYAGPILYN

SEQ ID NO: 205-206 - skipped (2 entries)

SEQ ID NO: 207 - protein, 7 aa, synthetic construct
  REGION 1..7: note = Targeting peptide sequence
RPLPPLP

SEQ ID NO: 208 - protein, 7 aa, synthetic construct
  REGION 1..7: note = Targeting peptide sequence
APPLPPR

SEQ ID NO: 209 - protein, 12 aa, synthetic construct
  REGION 1..12: note = Targeting peptide sequence
DVFYPYPYASGS

SEQ ID NO: 210 - protein, 6 aa, synthetic construct
  REGION 1..6: note = Targeting peptide sequence
MYWYPY

SEQ ID NO: 211 - protein, 12 aa, synthetic construct
  REGION 1..12: note = Targeting peptide sequence
DITWDQLWDLMK

SEQ ID NO: 212 - protein, 8 aa, synthetic construct
  REGION 1..8: note = Targeting peptide sequence
  SITE 5: note = MISC_FEATURE - Xaa can be G or L
CWDDXWLC

SEQ ID NO: 213 - protein, 14 aa, synthetic construct
  REGION 1..14: note = Targeting peptide sequence
EWCEYLGGYLRCYA

SEQ ID NO: 214 - protein, 14 aa, synthetic construct
  REGION 1..14: note = Targeting peptide sequence
  SITE 2: note = misc_feature - Xaa can be any naturally occurring amino acid
  REGION 4..5: note = misc_feature - Xaa can be any naturally occurring amino acid
  SITE 8: note = misc_feature - Xaa can be any naturally occurring amino acid
  SITE 11: note = misc_feature - Xaa can be any naturally occurring amino acid
  SITE 13: note = misc_feature - Xaa can be any naturally occurring amino acid
YXCXXGPXTWXCXP

SEQ ID NO: 215 - protein, 14 aa, synthetic construct
  REGION 1..14: note = Targeting peptide sequence
IEGPTLRQWLAARA

SEQ ID NO: 216 - skipped

SEQ ID NO: 217 - protein, 7 aa, synthetic construct
  REGION 1..7: note = Targeting peptide sequence
  SITE 1: note = misc_feature - Xaa can be any naturally occurring amino acid
  REGION 3..4: note = misc_feature - Xaa can be any naturally occurring amino acid
XFXXYLW

SEQ ID NO: 218 - protein, 13 aa, synthetic construct
  REGION 1..13: note = Targeting peptide sequence
SSIISHFRWGLCD

SEQ ID NO: 219 - protein, 13 aa, synthetic construct
  REGION 1..13: note = Targeting peptide sequence
MSRPACPPNDKYE

SEQ ID NO: 220 - protein, 8 aa, synthetic construct
  REGION 1..8: note = Targeting peptide sequence
CLRSGRGC

SEQ ID NO: 221 - protein, 9 aa, synthetic construct
  REGION 1..9: note = Targeting peptide sequence
CHWMFSPWC

SEQ ID NO: 222 - skipped

SEQ ID NO: 223 - protein, 8 aa, synthetic construct
  REGION 1..8: note = Targeting peptide sequence
CSSRLDAC

SEQ ID NO: 224 - protein, 7 aa, synthetic construct
  REGION 1..7: note = Targeting peptide sequence
CLPVASC

SEQ ID NO: 225 - protein, 13 aa, synthetic construct
  REGION 1..13: note = Targeting peptide sequence
CGFECVRQCPERC

SEQ ID NO: 226 - protein, 13 aa, synthetic construct
  REGION 1..13: note = Targeting peptide sequence
CVALCREACGEGC

SEQ ID NO: 227 - protein, 9 aa, synthetic construct
  REGION 1..9: note = Targeting peptide sequence
SWCEPGWCR

SEQ ID NO: 228 - protein, 7 aa, synthetic construct
  REGION 1..7: note = Targeting peptide sequence
YSGKWGW

SEQ ID NO: 229 - protein, 7 aa, synthetic construct
  REGION 1..7: note = Targeting peptide sequence
GLSGGRS

SEQ ID NO: 230 - protein, 7 aa, synthetic construct
  REGION 1..7: note = Targeting peptide sequence
LMLPRAD

SEQ ID NO: 231 - protein, 9 aa, synthetic construct
  REGION 1..9: note = Targeting peptide sequence
CSCFRDVCC

SEQ ID NO: 232 - protein, 9 aa, synthetic construct
  REGION 1..9: note = Targeting peptide sequence
CRDVVSVIC

SEQ ID NO: 233 - protein, 5 aa, synthetic construct
  REGION 1..5: note = Targeting peptide sequence
CNGRC

SEQ ID NO: 234 - protein, 6 aa, synthetic construct
  REGION 1..6: note = Targeting peptide sequence
MARSGL

SEQ ID NO: 235 - protein, 6 aa, synthetic construct
  REGION 1..6: note = Targeting peptide sequence
MARAKE

SEQ ID NO: 236 - protein, 6 aa, synthetic construct
  REGION 1..6: note = Targeting peptide sequence
MSRTMS

SEQ ID NO: 237 - protein, 6 aa, synthetic construct
  REGION 1..6: note = Targeting peptide sequence
KCCYSL

SEQ ID NO: 238 - protein, 14 aa, synthetic construct
  REGION 1..14: note = Targeting peptide sequence
MYWGDSHWLQYWYE

SEQ ID NO: 239 - protein, 7 aa, synthetic construct
  REGION 1..7: note = Targeting peptide sequence
MQLPLAT

SEQ ID NO: 240 - protein, 4 aa, synthetic construct
  REGION 1..4: note = Targeting peptide sequence
EWLS

SEQ ID NO: 241 - protein, 4 aa, synthetic construct
  REGION 1..4: note = Targeting peptide sequence
SNEW

SEQ ID NO: 242 - protein, 4 aa, synthetic construct
  REGION 1..4: note = Targeting peptide sequence
TNYL

SEQ ID NO: 243 - protein, 8 aa, synthetic construct
  REGION 1..8: note = Targeting peptide sequence
WIFPWIQL

SEQ ID NO: 244 - protein, 12 aa, synthetic construct
  REGION 1..12: note = Targeting peptide sequence
WDLAWMFRLPVG

SEQ ID NO: 245 - protein, 13 aa, synthetic construct
  REGION 1..13: note = Targeting peptide sequence
CTVALPGGYVRVC

SEQ ID NO: 246 - protein, 9 aa, synthetic construct
  REGION 1..9: note = Targeting peptide sequence
CVPELGHEC

SEQ ID NO: 247 - protein, 9 aa, synthetic construct
  REGION 1..9: note = Targeting peptide sequence
CGRRAGGSC

SEQ ID NO: 248 - protein, 13 aa, synthetic construct
  REGION 1..13: note = Targeting peptide sequence
CVAYCIEHHCWTC

SEQ ID NO: 249 - protein, 12 aa, synthetic construct
  REGION 1..12: note = Targeting peptide sequence
CVFAHNYDYLVC

SEQ ID NO: 250 - protein, 10 aa, synthetic construct
  REGION 1..10: note = Targeting peptide sequence
CVFTSNYAFC

SEQ ID NO: 251 - protein, 7 aa, synthetic construct
  REGION 1..7: note = Targeting peptide sequence
VHSPNKK

SEQ ID NO: 252 - protein, 9 aa, synthetic construct
  REGION 1..9: note = Targeting peptide sequence
CDCRGDCFC

SEQ ID NO: 253 - protein, 7 aa, synthetic construct
  REGION 1..7: note = Targeting peptide sequence
CRGDGWC

SEQ ID NO: 254 - protein, 6 aa, synthetic construct
  REGION 1..6: note = Targeting peptide sequence
  SITE 1: note = misc_feature - Xaa can be any naturally occurring amino acid
  SITE 6: note = misc_feature - Xaa can be any naturally occurring amino acid
XRGCDX

SEQ ID NO: 255 - skipped

SEQ ID NO: 256 - protein, 10 aa, synthetic construct
  REGION 1..10: note = Targeting peptide sequence
CTTHWGFTLC

SEQ ID NO: 257 - protein, 11 aa, synthetic construct
  REGION 1..11: note = Targeting peptide sequence
SGKGPRQITAL

SEQ ID NO: 258 - skipped

SEQ ID NO: 259 - protein, 6 aa, synthetic construct
  REGION 1..6: note = Targeting peptide sequence
VYMSPF

SEQ ID NO: 260 - protein, 7 aa, synthetic construct
  REGION 1..7: note = Targeting peptide sequence
MQLPLAT

SEQ ID NO: 261 - protein, 7 aa, synthetic construct
  REGION 1..7: note = Targeting peptide sequence
ATWLPPR

SEQ ID NO: 262 - protein, 12 aa, synthetic construct
  REGION 1..12: note = Targeting peptide sequence
HTMYYHHYQHHL

SEQ ID NO: 263 - protein, 19 aa, synthetic construct
  REGION 1..19: note = Targeting peptide sequence
SEVGCRAGPLQWLCEKYFG

SEQ ID NO: 264 - protein, 18 aa, synthetic construct
  REGION 1..18: note = Targeting peptide sequence
CGLLPVGRPDRNVWRWLC

SEQ ID NO: 265 - protein, 15 aa, synthetic construct
  REGION 1..15: note = Targeting peptide sequence
CKGQCDRFKGLPWEC

SEQ ID NO: 266 - protein, 5 aa, synthetic construct
  REGION 1..5: note = Targeting peptide sequence
SGRSA

SEQ ID NO: 267 - protein, 4 aa, synthetic construct
  REGION 1..4: note = Targeting peptide sequence
WGFP

SEQ ID NO: 268 - protein, 7 aa, synthetic construct
  REGION 1..7: note = Targeting peptide sequence
  SITE 1: note = misc_feature - Xaa can be any naturally occurring amino acid
  REGION 3..4: note = misc_feature - Xaa can be any naturally occurring amino acid
XFXXYLW

SEQ ID NO: 269 - protein, 17 aa, synthetic construct
  REGION 1..17: note = Targeting peptide sequence
AEPMPHSLNFSQYLWYT

SEQ ID NO: 270 - protein, 6 aa, synthetic construct
  REGION 1..6: note = Targeting peptide sequence
  SITE 4: note = MISC_FEATURE - Xaa can be W or F
WAYXSP

SEQ ID NO: 271 - protein, 7 aa, synthetic construct
  REGION 1..7: note = Targeting peptide sequence
IELLQAR

SEQ ID NO: 272 - protein, 12 aa, synthetic construct
  REGION 1..12: note = Targeting peptide sequence
DITWDQLWDLMK

SEQ ID NO: 273 - protein, 16 aa, synthetic construct
  REGION 1..16: note = Targeting peptide sequence
AYTKCSRQWRTCMTTH

SEQ ID NO: 274 - protein, 15 aa, synthetic construct
  REGION 1..15: note = Targeting peptide sequence
PQNSKIPGPTFLDPH

SEQ ID NO: 275 - protein, 15 aa, synthetic construct
  REGION 1..15: note = Targeting peptide sequence
SMEPALPDWWWKMFK

SEQ ID NO: 276 - protein, 16 aa, synthetic construct
  REGION 1..16: note = Targeting peptide sequence
ANTPCGPYTHDCPVKR

SEQ ID NO: 277 - protein, 12 aa, synthetic construct
  REGION 1..12: note = Targeting peptide sequence
TACHQHVRMVRP

SEQ ID NO: 278 - protein, 12 aa, synthetic construct
  REGION 1..12: note = Targeting peptide sequence
VPWMEPAYQRFL

SEQ ID NO: 279 - protein, 8 aa, synthetic construct
  REGION 1..8: note = Targeting peptide sequence
DPRATPGS

SEQ ID NO: 280 - protein, 12 aa, synthetic construct
  REGION 1..12: note = Targeting peptide sequence
FRPNRAQDYNTN

SEQ ID NO: 281 - protein, 9 aa, synthetic construct
  REGION 1..9: note = Targeting peptide sequence
CTKNSYLMC

SEQ ID NO: 282 - protein, 11 aa, synthetic construct
  REGION 1..11: note = Targeting peptide sequence
  SITE 2: note = MISC_FEATURE - Xaa can be R or Q
  SITE 3: note = MISC_FEATURE - Xaa can be L or R
  SITE 5: note = MISC_FEATURE - Xaa can be G or N
  REGION 6..7: note = misc_feature - Xaa can be any naturally occurring amino acid
  SITE 9: note = MISC_FEATURE - Xaa can be A or V
CXXTXXXGXGC

SEQ ID NO: 283 - protein, 9 aa, synthetic construct
  REGION 1..9: note = Targeting peptide sequence
CPIEDRPMC

SEQ ID NO: 284 - protein, 12 aa, synthetic construct
  REGION 1..12: note = Targeting peptide sequence
HEWSYLAPYPWF

SEQ ID NO: 285 - protein, 9 aa, synthetic construct
  REGION 1..9: note = Targeting peptide sequence
MCPKHPLGC

SEQ ID NO: 286 - protein, 15 aa, synthetic construct
  REGION 1..15: note = Targeting peptide sequence
RMWPSSTVNLSAGRR

SEQ ID NO: 287 - protein, 20 aa, synthetic construct
  REGION 1..20: note = Targeting peptide sequence
SAKTAVSQRVWLPSHRGGEP

SEQ ID NO: 288 - protein, 20 aa, synthetic construct
  REGION 1..20: note = Targeting peptide sequence
KSREHVNNSACPSKRITAAL

SEQ ID NO: 289 - protein, 4 aa, synthetic construct
  REGION 1..4: note = Targeting peptide sequence
EGFR

SEQ ID NO: 290 - protein, 6 aa, synthetic construct
  REGION 1..6: note = Targeting peptide sequence
AGLGVR

SEQ ID NO: 291 - protein, 15 aa, synthetic construct
  REGION 1..15: note = Targeting peptide sequence
GTRQGHTMRLGVSDG

SEQ ID NO: 292 - protein, 15 aa, synthetic construct
  REGION 1..15: note = Targeting peptide sequence
IAGLATPGWSHWLAL

SEQ ID NO: 293 - protein, 7 aa, synthetic construct
  REGION 1..7: note = Targeting peptide sequence
SMSIARL

SEQ ID NO: 294 - protein, 7 aa, synthetic construct
  REGION 1..7: note = Targeting peptide sequence
HTFEPGV

SEQ ID NO: 295 - protein, 15 aa, synthetic construct
  REGION 1..15: note = Targeting peptide sequence
NTSLKRISNKRIRRK

SEQ ID NO: 296 - protein, 15 aa, synthetic construct
  REGION 1..15: note = Targeting peptide sequence
LRIKRKRRKRKKTRK

SEQ ID NO: 297 - protein, 10 aa, synthetic construct
  REGION 1..10: note = AAV capsid protein common antigenic motif (CAM) substitution sequence
CNDEMQVQVN

SEQ ID NO: 298 - protein, 10 aa, synthetic construct
  REGION 1..10: note = AAV capsid protein common antigenic motif (CAM) substitution sequence
SPDIVYADVC

SEQ ID NO: 299 - protein, 10 aa, synthetic construct
  REGION 1..10: note = AAV capsid protein common antigenic motif (CAM) substitution sequence
LDDCHNIDVN

SEQ ID NO: 300 - protein, 10 aa, synthetic construct
  REGION 1..10: note = AAV capsid protein common antigenic motif (CAM) substitution sequence
SCDCVTNSVS

SEQ ID NO: 301 - protein, 10 aa, synthetic construct
  REGION 1..10: note = AAV capsid protein common antigenic motif (CAM) substitution sequence
TVDSNPYEVN

SEQ ID NO: 302 - protein, 10 aa, synthetic construct
  REGION 1..10: note = AAV capsid protein common antigenic motif (CAM) substitution sequence
GDDHPNPDVL

SEQ ID NO: 303 - protein, 7 aa, adeno-associated virus
SASTGAS

SEQ ID NO: 304 - protein, 10 aa, adeno-associated virus
VFMIPQYGYL

SEQ ID NO: 305 - protein, 9 aa, adeno-associated virus
NQSGSAQNK

SEQ ID NO: 306 - protein, 8 aa, adeno-associated virus
KTDNNNSN

SEQ ID NO: 307 - protein, 7 aa, adeno-associated virus
KDDEDKF

SEQ ID NO: 308 - protein, 6 aa, adeno-associated virus
SAGASN

SEQ ID NO: 309 - protein, 10 aa, adeno-associated virus
STDPATGDVH

SEQ ID NO: 310 - protein, 7 aa, adeno-associated virus
DNNGLYT

SEQ ID NO: 311 - protein, 6 aa, adeno-associated virus
SQSGAS

SEQ ID NO: 312 - protein, 10 aa, adeno-associated virus
VFMVPQYGYL

SEQ ID NO: 313 - protein, 9 aa, adeno-associated virus
TPSGTTTQS

SEQ ID NO: 314 - protein, 8 aa, adeno-associated virus
SADNNNSE

SEQ ID NO: 315 - protein, 7 aa, adeno-associated virus
KDDEEKF

SEQ ID NO: 316 - protein, 6 aa, adeno-associated virus
GSEKTN

SEQ ID NO: 317 - protein, 10 aa, adeno-associated virus
NRQAATADVN

SEQ ID NO: 318 - protein, 7 aa, adeno-associated virus
DTNGVYS

SEQ ID NO: 319 - protein, 6 aa, adeno-associated virus
SQSGAS

SEQ ID NO: 320 - protein, 10 aa, adeno-associated virus
VFMVPQYGYL

SEQ ID NO: 321 - protein, 9 aa, adeno-associated virus
TTSGTTNQS

SEQ ID NO: 322 - protein, 8 aa, adeno-associated virus
ANDNNNSN

SEQ ID NO: 323 - protein, 7 aa, adeno-associated virus
KDDEEKF

SEQ ID NO: 324 - protein, 6 aa, adeno-associated virus
GTTASN

SEQ ID NO: 325 - protein, 10 aa, adeno-associated virus
NTAPTTGTVN

SEQ ID NO: 326 - protein, 7 aa, adeno-associated virus
DTNGVYS

SEQ ID NO: 327 - protein, 8 aa, adeno-associated virus
RLGESLQS

SEQ ID NO: 328 - protein, 10 aa, adeno-associated virus
VFMVPQYGYC

SEQ ID NO: 329 - protein, 9 aa, adeno-associated virus
GTTLNAGTA

SEQ ID NO: 330 - protein, 12 aa, adeno-associated virus
ANQNYKIPATGS

SEQ ID NO: 331 - protein, 7 aa, adeno-associated virus
GPADSKF

SEQ ID NO: 332 - protein, 6 aa, adeno-associated virus
QNGNTA

SEQ ID NO: 333 - protein, 10 aa, adeno-associated virus
SNLPTVDRLT

SEQ ID NO: 334 - protein, 7 aa, adeno-associated virus
DAAGKYT

SEQ ID NO: 335 - protein, 10 aa, adeno-associated virus
EIKSGSVDGS

SEQ ID NO: 336 - protein, 10 aa, adeno-associated virus
VFTLPQYGYA

SEQ ID NO: 337 - protein, 9 aa, adeno-associated virus
STNNTGGVQ

SEQ ID NO: 338 - protein, 7 aa, adeno-associated virus
SGVNRAS

SEQ ID NO: 339 - protein, 7 aa, adeno-associated virus
LQGSNTY

SEQ ID NO: 340 - protein, 8 aa, adeno-associated virus
ANPGTTAT

SEQ ID NO: 341 - protein, 10 aa, adeno-associated virus
TTAPATGTYN

SEQ ID NO: 342 - protein, 7 aa, adeno-associated virus
DSTGEYR

SEQ ID NO: 343 - protein, 7 aa, adeno-associated virus
SASTGAS

SEQ ID NO: 344 - protein, 10 aa, adeno-associated virus
VFMIPQYGYL

SEQ ID NO: 345 - protein, 9 aa, adeno-associated virus
NQSGSAQNK

SEQ ID NO: 346 - protein, 8 aa, adeno-associated virus
KTDNNNSN

SEQ ID NO: 347 - protein, 7 aa, adeno-associated virus
KDDKDKF

SEQ ID NO: 348 - protein, 6 aa, adeno-associated virus
SAGASN

SEQ ID NO: 349 - protein, 10 aa, adeno-associated virus
STDPATGDVH

SEQ ID NO: 350 - protein, 7 aa, adeno-associated virus
DNNGLYT

SEQ ID NO: 351 - protein, 7 aa, adeno-associated virus
SETAGST

SEQ ID NO: 352 - protein, 10 aa, adeno-associated virus
VFMIPQYGYL

SEQ ID NO: 353 - protein, 9 aa, adeno-associated virus
NPGGTAGNR

SEQ ID NO: 354 - protein, 8 aa, adeno-associated virus
LDQNNNSN

SEQ ID NO: 355 - protein, 7 aa, adeno-associated virus
KDDEDRF

SEQ ID NO: 356 - protein, 6 aa, adeno-associated virus
GATNKT

SEQ ID NO: 357 - protein, 10 aa, adeno-associated virus
NTAAQTQVVN

SEQ ID NO: 358 - protein, 7 aa, adeno-associated virus
DSQGVYS

SEQ ID NO: 359 - protein, 8 aa, adeno-associated virus
NGTSGGAT

SEQ ID NO: 360 - protein, 10 aa, adeno-associated virus
VFMIPQYGYL

SEQ ID NO: 361 - protein, 9 aa, adeno-associated virus
TTGGTANTQ

SEQ ID NO: 362 - protein, 8 aa, adeno-associated virus
TGQNNNSN

SEQ ID NO: 363 - protein, 7 aa, adeno-associated virus
KDDEERF

SEQ ID NO: 364 - protein, 6 aa, adeno-associated virus
NAARDN

SEQ ID NO: 365 - protein, 11 aa, adeno-associated virus
NTAPQIGTVNS

SEQ ID NO: 366 - protein, 7 aa, adeno-associated virus
NTEGVYS

SEQ ID NO: 367 - protein, 8 aa, adeno-associated virus
NSTSGGSS

SEQ ID NO: 368 - protein, 10 aa, adeno-associated virus
VFMIPQYGYL

SEQ ID NO: 369 - protein, 9 aa, adeno-associated virus
INGSGQNQQ

SEQ ID NO: 370 - protein, 8 aa, adeno-associated virus
VTQNNNSE

SEQ ID NO: 371 - protein, 7 aa, adeno-associated virus
KEGEDRF

SEQ ID NO: 372 - protein, 6 aa, adeno-associated virus
GTGRDN

SEQ ID NO: 373 - protein, 10 aa, adeno-associated virus
QAQAQTGWVQ

SEQ ID NO: 374 - protein, 7 aa, adeno-associated virus
NTEGVYS

SEQ ID NO: 375 - protein, 8 aa, adeno-associated virus
NGTSGGST

SEQ ID NO: 376 - protein, 10 aa, adeno-associated virus
VFMVPQYGYL

SEQ ID NO: 377 - protein, 9 aa, adeno-associated virus
QTTGTGGTQ

SEQ ID NO: 378 - protein, 8 aa, adeno-associated virus
TNQNNNSN

SEQ ID NO: 379 - protein, 7 aa, adeno-associated virus
KDDDDRF

SEQ ID NO: 380 - protein, 6 aa, adeno-associated virus
GAGNDG

SEQ ID NO: 381 - protein, 10 aa, adeno-associated virus
NTQAQTGLVH

SEQ ID NO: 382 - protein, 7 aa, adeno-associated virus
NTEGVYS

SEQ ID NO: 383 - protein, 8 aa, adeno-associated virus
NGTSGGST

SEQ ID NO: 384 - protein, 10 aa, adeno-associated virus
VFMIPQYGYL

SEQ ID NO: 385 - protein, 9 aa, adeno-associated virus
STGGTAGTQ

SEQ ID NO: 386 - protein, 8 aa, adeno-associated virus
LSQNNNSN

SEQ ID NO: 387 - protein, 7 aa, adeno-associated virus
KDDEERF

SEQ ID NO: 388 - protein, 6 aa, adeno-associated virus
GAGKDN

SEQ ID NO: 389 - protein, 10 aa, adeno-associated virus
NAAPIVGAVN

SEQ ID NO: 390 - protein, 7 aa, adeno-associated virus
NTDGTYS

SEQ ID NO: 391 - protein, 8 aa, adeno-associated virus
NGTSGGST

SEQ ID NO: 392 - protein, 10 aa, adeno-associated virus
VFMIPQYGYL

SEQ ID NO: 393 - protein, 9 aa, adeno-associated virus
STGGTQGTQ

SEQ ID NO: 394 - protein, 8 aa, adeno-associated virus
LSQNNNSN

SEQ ID NO: 395 - protein, 7 aa, adeno-associated virus
KDDEERF

SEQ ID NO: 396 - protein, 6 aa, adeno-associated virus
GAGRDN

SEQ ID NO: 397 - protein, 10 aa, adeno-associated virus
NTGPIVGNVN

SEQ ID NO: 398 - protein, 7 aa, adeno-associated virus
NTEGTYS

SEQ ID NO: 399 - protein, 8 aa, adeno-associated virus
RLGTTSSS

SEQ ID NO: 400 - protein, 10 aa, adeno-associated virus
VFMVPQYGYC

SEQ ID NO: 401 - protein, 9 aa, adeno-associated virus
GETLNQGNA

SEQ ID NO: 402 - protein, 12 aa, adeno-associated virus
ASQNYKIPASGG

SEQ ID NO: 403 - protein, 7 aa, adeno-associated virus
GPSDGDF

SEQ ID NO: 404 - protein, 6 aa, adeno-associated virus
VTGNTT

SEQ ID NO: 405 - protein, 10 aa, adeno-associated virus
TTAPITGNVT

SEQ ID NO: 406 - protein, 7 aa, adeno-associated virus
DTTGKYT

SEQ ID NO: 407 - protein, 8 aa, adeno-associated virus
RIGTTANS

SEQ ID NO: 408 - protein, 10 aa, adeno-associated virus
VFMVPQYGYC

SEQ ID NO: 409 - protein, 9 aa, adeno-associated virus
GNSLNQGTA

SEQ ID NO: 410 - protein, 12 aa, adeno-associated virus
ANQNYKIPASGG

SEQ ID NO: 411 - protein, 7 aa, adeno-associated virus
GAGDSDF

SEQ ID NO: 412 - protein, 6 aa, adeno-associated virus
PSGNTT

SEQ ID NO: 413 - protein, 10 aa, adeno-associated virus
TTAPHIANLD

SEQ ID NO: 414 - protein, 7 aa, adeno-associated virus
DNAGNYH

SEQ ID NO: 415 - protein, 8 aa, adeno-associated virus
RLGTTSNS

SEQ ID NO: 416 - protein, 10 aa, adeno-associated virus
VFMVPQYGYC

SEQ ID NO: 417 - protein, 9 aa, adeno-associated virus
GETLNQGNA

SEQ ID NO: 418 - protein, 12 aa, adeno-associated virus
ASQNYKIPASGG

SEQ ID NO: 419 - protein, 7 aa, adeno-associated virus
GPSDGDF

SEQ ID NO: 420 - protein, 6 aa, adeno-associated virus
VTGNTT

SEQ ID NO: 421 - protein, 10 aa, adeno-associated virus
TTAPITGNVT

SEQ ID NO: 422 - protein, 7 aa, adeno-associated virus
DTTGKYT

SEQ ID NO: 423 - protein, 8 aa, adeno-associated virus
RLGSSNAS

SEQ ID NO: 424 - protein, 10 aa, adeno-associated virus
VFMVPQYGYC

SEQ ID NO: 425 - protein, 9 aa, adeno-associated virus
GGTLNQGNS

SEQ ID NO: 426 - protein, 12 aa, adeno-associated virus
ASQNYKIPQGRN

SEQ ID NO: 427 - protein, 7 aa, adeno-associated virus
ANDATDF

SEQ ID NO: 428 - protein, 6 aa, adeno-associated virus
ITGNTT

SEQ ID NO: 429 - protein, 10 aa, adeno-associated virus
TTVPTVDDVD

SEQ ID NO: 430 - protein, 7 aa, adeno-associated virus
DNAGAYK

SEQ ID NO: 431 - protein, 8 aa, adeno-associated virus
RIQGPSGG

SEQ ID NO: 432 - protein, 10 aa, adeno-associated virus
IYTIPQYGYC

SEQ ID NO: 433 - protein, 9 aa, adeno-associated virus
VSQAGSSGR

SEQ ID NO: 434 - protein, 12 aa, adeno-associated virus
ASNITKNNVFSV

SEQ ID NO: 435 - protein, 7 aa, adeno-associated virus
FSGEPDR

SEQ ID NO: 436 - protein, 8 aa, adeno-associated virus
VYDQTTAT

SEQ ID NO: 437 - protein, 10 aa, adeno-associated virus
VTPGTRAAVN

SEQ ID NO: 438 - protein, 7 aa, adeno-associated virus
SDTGSYS

SEQ ID NO: 439 - DNA, 53 bp, synthetic construct
  misc_feature 1..53: note = Radomized AAV1e common antigenic motif coding sequence
  source 1..53: mol_type = other DNA
ctcaaatcagtccggaagtgggggggggggggacttgctgtttagccgtgggt

SEQ ID NO: 440 - DNA, 55 bp, synthetic construct
  misc_feature 1..55: note = Radomized AAV1e common antigenic motif coding sequence
  source 1..55: mol_type = other DNA
agcgcgtttctaaatggggggggggggggggggggggtaattttacctggactgg

SEQ ID NO: 441 - DNA, 55 bp, synthetic construct
  misc_feature 1..55: note = Radomized AAV1e common antigenic motif coding sequence
  source 1..55: mol_type = other DNA
tcaatttccagagcagctttatggacttattattattatggtgatggctatggga

SEQ ID NO: 442 - DNA, 57 bp, synthetic construct
  misc_feature 1..57: note = Radomized AAV1e common antigenic motif coding sequence
  source 1..57: mol_type = other DNA
aatctgttgttgttgattttactgtgaataatgataataataagaatgagcctcgcc

SEQ ID NO: 443-476 - skipped (34 entries)

SEQ ID NO: 477 - DNA, 25 bp, synthetic construct
  misc_feature 1..25: note = qPCR primer sequence
  source 1..25: mol_type = other DNA
aacatgctacgcagagagggagtgg

SEQ ID NO: 478 - DNA, 29 bp, synthetic construct
  misc_feature 1..29: note = qPCR primer sequence
  source 1..29: mol_type = other DNA
catgagacaaggaacccctagtgatggag

SEQ ID NO: 479 - DNA, 54 bp, synthetic construct
  misc_feature 1..54: note = AAV CAM library amplification primer sequence
  misc_feature 25..29: note = n is a, c, g, or t
  source 1..54: mol_type = other DNA
ccctacacgacgctcttccgatctnnnnncagaactcaaaatcagtccggaagt

SEQ ID NO: 480 - DNA, 58 bp, synthetic construct
  misc_feature 1..58: note = AAV CAM library amplification primer sequence
  misc_feature 33..37: note = n is a, c, g, or t
  source 1..58: mol_type = other DNA
gactggagttcagacgtgtgctcttccgatctnnnnngccaggtaatgctcccatagc

SEQ ID NO: 481 - DNA, 22 bp, synthetic construct
  misc_feature 1..22: note = qPCR primer sequence
  source 1..22: mol_type = other DNA
ccttcgcttcaaaaaatggaac

SEQ ID NO: 482 - DNA, 24 bp, synthetic construct
  misc_feature 1..24: note = qPCR primer sequence
  source 1..24: mol_type = other DNA
aaaagcactctgattgacaaatac

SEQ ID NO: 483 - protein, 4 aa, adeno-associated virus
AQNK

SEQ ID NO: 484 - protein, 7 aa, adeno-associated virus
KTDNNNS

SEQ ID NO: 485 - protein, 8 aa, synthetic construct
  REGION 1..8: note = AAV capsid protein common antigenic motif (CAM) substitution sequence
TPGGNATR

SEQ ID NO: 486 - protein, 9 aa, synthetic construct
  REGION 1..9: note = AAV capsid protein common antigenic motif (CAM) substitution
TADHDTKGV

SEQ ID NO: 487 - protein, 10 aa, synthetic construct
  REGION 1..10: note = AAV capsid protein common antigenic motif (CAM) substitution
DLDPKATEVE

SEQ ID NO: 488 - protein, 6 aa, synthetic construct
  REGION 1..6: note = AAV capsid protein common antigenic motif (CAM) substitution sequence
SNGRGV

SEQ ID NO: 489 - protein, 7 aa, synthetic construct
  REGION 1..7: note = AAV capsid protein common antigenic motif (CAM) substitution sequence
VNTSLVG

SEQ ID NO: 490 - protein, 7 aa, synthetic construct
  REGION 1..7: note = AAV capsid protein common antigenic motif (CAM) substitution sequence
IRGAGAV

SEQ ID NO: 491 - protein, 7 aa, synthetic construct
  REGION 1..7: note = AAV capsid protein common antigenic motif (CAM) substitution sequence
YPGGNYK

SEQ ID NO: 492 - protein, 6 aa, synthetic construct
  REGION 1..6: note = AAV capsid protein common antigenic motif (CAM) substitution sequence
KQKNVN

SEQ ID NO: 493 - protein, 6 aa, synthetic construct
  REGION 1..6: note = AAV capsid protein common antigenic motif (CAM) substitution sequence
RMSSIK

SEQ ID NO: 494 - DNA, 221 bp, synthetic construct
  misc_feature 1..221: note = AAV1e
  source 1..221: mol_type = other DNA
ctcaaatcagtccggaagtgggggggggggggacttgctgtttagccgtgggtagcgcgtttctaaatggggggggggggggggggggggtaattttacctggactggtcaatttccagagcagctttatggactttattattattatggtgatggctatgggaaatctgttgttgttgattttactgtgaataatgataataataagaatgagcctcgcc

SEQ ID NO: 495 - protein, 4 aa, synthetic construct
  REGION 1..4: note = Evolved library amino acid sequence
TGPR

SEQ ID NO: 496 - protein, 4 aa, synthetic construct
  REGION 1..4: note = Evolved library amino acid sequence
SRGT

SEQ ID NO: 497 - protein, 4 aa, synthetic construct
  REGION 1..4: note = Evolved library amino acid sequence
QGFR

SEQ ID NO: 498 - protein, 4 aa, synthetic construct
  REGION 1..4: note = Evolved library amino acid sequence
RPGG

SEQ ID NO: 499 - protein, 4 aa, synthetic construct
  REGION 1..4: note = Evolved library amino acid sequence
LASR

SEQ ID NO: 500 - protein, 4 aa, synthetic construct
  REGION 1..4: note = Evolved library amino acid sequence
LRDR

SEQ ID NO: 501 - protein, 8 aa, synthetic construct
  REGION 1..8: note = Evolved library amino acid sequence
KPGGNATR

SEQ ID NO: 502 - protein, 8 aa, synthetic construct
  REGION 1..8: note = Evolved library amino acid sequence
TKTDNNNS

SEQ ID NO: 503 - protein, 8 aa, synthetic construct
  REGION 1..8: note = Evolved library amino acid sequence
TSGGNATR

SEQ ID NO: 504 - protein, 7 aa, synthetic construct
  REGION 1..7: note = Evolved library amino acid sequence
TPGVMRR

SEQ ID NO: 505 - protein, 8 aa, synthetic construct
  REGION 1..8: note = Evolved library amino acid sequence
TPGGNAPR

SEQ ID NO: 506 - protein, 8 aa, synthetic construct
  REGION 1..8: note = Evolved library amino acid sequence
TTGGNATR

SEQ ID NO: 507 - protein, 8 aa, synthetic construct
  REGION 1..8: note = Evolved library amino acid sequence
TPGGNAKR

SEQ ID NO: 508 - protein, 8 aa, synthetic construct
  REGION 1..8: note = Evolved library amino acid sequence
MPGGNATR

SEQ ID NO: 509 - protein, 8 aa, synthetic construct
  REGION 1..8: note = Evolved library amino acid sequence
TPRGNATR

SEQ ID NO: 510 - protein, 10 aa, synthetic construct
  REGION 1..10: note = Evolved library amino acid sequence
TKDPSNSPVY

SEQ ID NO: 511 - protein, 10 aa, synthetic construct
  REGION 1..10: note = Evolved library amino acid sequence
SKDTNSVNVF

SEQ ID NO: 512 - protein, 10 aa, synthetic construct
  REGION 1..10: note = Evolved library amino acid sequence
TKDPEQYAVQ

SEQ ID NO: 513 - protein, 10 aa, synthetic construct
  REGION 1..10: note = Evolved library amino acid sequence
TKDVKMDNVF

SEQ ID NO: 514 - protein, 10 aa, synthetic construct
  REGION 1..10: note = Evolved library amino acid sequence
TADHDTKEVL

SEQ ID NO: 515 - protein, 10 aa, synthetic construct
  REGION 1..10: note = Evolved library amino acid sequence
SVDPDSKHVL

SEQ ID NO: 516 - protein, 10 aa, synthetic construct
  REGION 1..10: note = Evolved library amino acid sequence
DTDARNQAVF

SEQ ID NO: 517 - protein, 10 aa, synthetic construct
  REGION 1..10: note = Evolved library amino acid sequence
STGPATGDVH

SEQ ID NO: 518 - protein, 10 aa, synthetic construct
  REGION 1..10: note = Evolved library amino acid sequence
DSDSPTNNVV

SEQ ID NO: 519 - protein, 10 aa, synthetic construct
  REGION 1..10: note = Evolved library amino acid sequence
ELDGQIGNVC

SEQ ID NO: 520 - protein, 10 aa, synthetic construct
  REGION 1..10: note = Evolved library amino acid sequence
GVDVVNQPVV

SEQ ID NO: 521 - protein, 10 aa, synthetic construct
  REGION 1..10: note = Evolved library amino acid sequence
SADPATGDVH

SEQ ID NO: 522 - protein, 10 aa, synthetic construct
  REGION 1..10: note = Evolved library amino acid sequence
APDGGGRDVV

SEQ ID NO: 523 - protein, 10 aa, synthetic construct
  REGION 1..10: note = Evolved library amino acid sequence
DVDARIQDVF

SEQ ID NO: 524 - protein, 8 aa, synthetic construct
  REGION 1..8: note = amino acid sequence for lead variant
TPGGNATR

SEQ ID NO: 525 - protein, 9 aa, synthetic construct
  REGION 1..9: note = amino acid sequence for lead variant
TADHDTKGV

SEQ ID NO: 526 - protein, 10 aa, synthetic construct
  REGION 1..10: note = amino acid sequence for lead variant
DLDPKATEVE

SEQ ID NO: 527 - protein, 4 aa, synthetic construct
  REGION 1..4: note = amino acid sequence for lead variant
SERR